(19) United States
(12) Patent Application Publication
(10) Pub. No.: US 2025/0270566 A1
(43) Pub. Date: Aug. 28, 2025

(54) DUAL-SPECIFICITY RNA ATPAMERS FOR REGULATING O-GLCNACYLATION

(71) Applicant: University of Georgia Research Foundation, Inc., Athens, GA (US)

(72) Inventors: Gerald W. Hart, Athens, GA (US); Yi Zhu, Athens, GA (US)

(21) Appl. No.: 18/701,725

(22) PCT Filed: Nov. 11, 2022
(86) PCT No.: PCT/US2022/079729

§ 371 Date: Apr. 16, 2024

Related U.S. Application Data
(60) Provisional application No. 63/264,333, filed on Nov. 19, 2021.

(51) Int. Cl. C12N 15/115

(52) U.S. Cl. CPC C12N 15/115; C12N 2310/16

(57) ABSTRACT

Disclosed herein are dual-specificity (DS) aptamers involving modular designed RNA that connect two aptamer motifs with a linker domain. In cells, they induce proximity between O-GlcNAc transferase (OGT) and O-GlcNAcase (OGA) and a designated protein, and increase or decrease O-GlcNAcylation on the substrate. These RNA aptamers have short half-lives in cells and their binding can be controlled by riboswitches.

### CROSS-REFERENCE TO RELATED APPLICATIONS

This application claims benefit of U.S. Provisional Application No. 63/264,333, filed Nov. 19, 2021, which is hereby incorporated herein by reference in its entirety.

### STATEMENT REGARDING FEDERALLY SPONSORED RESEARCH OR DEVELOPMENT

This invention was made with Government Support under Grant Nos. HL107153 and GM116891 awarded by the National Institutes of Health. The Government has certain rights in the invention.

### SEQUENCE LISTING

This application contains a sequence listing filed in ST.26 format entitled “222105_2120_Sequence_Listing” created on Nov. 9, 2022. The content of the sequence listing is incorporated herein in its entirety.

### BACKGROUND

Wnt signaling plays essential roles in the regulation of cell proliferation, differentiation, and self-renewal of stem cells. Its abnormalities occur in many types of cancer and inheritable disorders (Clevers, H. Cell 2006 127:469-480; Nusse, R., et al. Cell 2017 169:985-999). β-catenin is the key transcription factor in the canonical Wnt signaling pathway. Its functions are regulated by post-translational modifications (PTMs). In resting cells, β-catenin maintains a low abundance due to the consecutive phosphorylation, ubiquitination, and degradation (Hart, M., et al. Current Biology 1999 9:207-211; Liu, C., et al. Cell 2002 108:837-847). Wnt signaling triggers a cascade of events that ultimately stabilizes β-catenin by inhibiting its ubiquitination, causing its nuclear accumulation and activation of downstream genes (Li, V. S., et al. Cell 2012 149:1245-1256). Besides phosphorylation and ubiquitination, the functions of β-catenin are regulated by PTMs including O-GlcNAcylation (Olivier-Van Stichelen, S., et al. FASEB J 28:3325-3338).

O-linked β-N-acetylglucosamine (O-GlcNAc) is an abundant PTM attached to serine and threonine residues of more than 8,000 human nucleocytoplasmic proteins (Torres, C. R., et al. J Biol Chem 1984 259:3308-3317; Hanover, J. A., et al. J Biol Chem 1987 262:9887-9894; Wulff-Fuentes, E., et al. Sci Data 2021 8:25). O-GlcNAc is a highly dynamic PTM which is actively added and removed by two enzymes: O-GlcNAc transferase (OGT) and O-GlcNAcase (OGA), respectively (Haltiwanger, R. S., et al. J Biol Chem 1992 267:9005-9013; Dong, D. L., et al. Journal of Biological Chemistry 1994 269:19321-19330). As a fast-cycling, nutrient-sensitive PTM, O-GlcNAc regulates many signaling events, interplays with phosphorylation, and regulates protein functions such as intermolecular interactions and degradation. Abnormal O-GlcNAcylation is associated with many human diseases, including cancer and diabetes (Hart, G. W., et al. Annu Rev Biochem 2011 80:825-858).

With current approaches, it is difficult to study the biological functions of O-GlcNAc on a specific protein. OGT and OGA are the sole O-GlcNAc modifying enzymes, any chemical inhibitors or genetic approaches targeting them change O-GlcNAcylation on thousands of proteins simultaneously. O-GlcNAc and phosphate frequently modify the same or proximal sites in a mutually exclusive way. This crosstalk makes studies that use site-directed mutagenesis to convert serine or threonine to other non-modifiable residues difficult to interpret. Moreover, there is no amino acid mimic of O-GlcNAcylation, as there is for phosphorylation. The lack of tools to modify O-GlcNAcylation of a single protein without affecting the many others in the cell has substantially impeded our understanding of the functions of this ubiquitous and essential modification.

To address this problem, some have used expressed protein ligation to study O-GlcNAc modified proteins in vitro (Tarrant, M. K., et al. Nat Chem Biol 2012 8:262-269; Marotta, N. P., et al. Nat Chem 2015 7:913-920). In this method, a chemically synthesized, O-GlcNAc modified peptide was ligated to bacterial expressed, unmodified protein portions to generate a full-length protein with O-GlcNAc on certain sites. Proteins produced in this method are completely modified at defined sites but can only be used for in vitro assays. It also requires cysteine residues at the ligation sites. Woo and colleagues used nanobody-tagged OGT or OGA constructs to selectively modify 0-GlcNAcylation of target proteins in cells (Ramirez, D. H., et al. ACS Chem Biol. 2020; Ge, Y., et al. Nat Chem Biol 2021 17:593-600). However, the tight and stable binding of these antibodies to the target proteins make sorting out the biological effects of altered O-GlcNAcylation from the effects of the antibody binding difficult.

### SUMMARY

Disclosed herein are dual-specificity (DS) aptamers involving modular designed RNA that connect two aptamer motifs with a linker domain. In cells, they induce proximity between O-GlcNAc transferase (OGT) and O-GlcNAcase (OGA) and a designated protein, and increase or decrease O-GlcNAcylation on the substrate. These RNA aptamers have short half-lives in cells and their binding can be controlled by riboswitches.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1J show a noninhibiting RNA aptamer targeting ncOGT generated from SELEX. FIG. 1A is a schematic of SELEX. FIGS. 1B and 1C show predicted secondary (FIG. 1B, SEQ ID NO:7) and tertiary (FIG. 1C) structures of T1. FIG. 1D shows radiolabeled Dot-Blot Assay measuring the binding affinity of T1 to full-length ncOGT. Data were fitted to a Michaelis-Menten model (curve). FIGS. 1E and 1F show Surface Plasmon Resonance (SPR) characterizing the binding affinity and kinetics of T1 to full-length ncOGT (FIG. 1E), and the TPR domain (FIG. 1F). Sensorgrams represent OGT concentrations from 1 μM to 977 μM (E) or 1 μM to 488 μM (F), in 2× serial dilutions. Data were fitted to a 1:1 binding model. FIG. 1G shows UDP-Glo assay of OGT activity in the presence of T1. T1 was included in the indicated reactions (orange) from 2 μM to 7.8 nM in 2× serial dilution. Data were normalized to the positive control reaction (red). One-way ANOVA test was performed. FIG. 1H shows RNA-IP and RT-qPCR quantification of OGT-bound T1 in cells. OGT was immunoprecipitated from lysate of T1-expressing cells, using buffers with Mg²⁺ (left) or without Mg²⁺ (right). Then the OGT-bound T1 was quantified by RT-qPCR. Data were normalized to the IgG groups. One-way ANOVA test was performed. RT—Reverse Transcriptase. N=3. FIG. 1I shows Western Blot (WB) analysis of global O-GlcNAcylation in HEK293T cells expressing T1 or AP3. N=3. FIG. 1J shows pulse-chase experiments measuring the half-life of T1 in cells. Cells stably expressing T1 were treated with ActD for the indicated periods. Abundance of T1 was quantified by RT-qPCR (left) and normalized to that of 18S rRNA (right) and initial timepoint (T=0). Data were fitted to a one-phase decay model (curve). N=3. Data are represented as mean±SD. ns, p≥0.05; ****, p<0.0001. See Table 2.

FIGS. 2A to 2L show DS aptamers increase O-GlcNAcylation on GFP-tagged proteins. FIG. 2A is a schematic of DS aptamers with flexible linkers (NL1F, T1-linker-AP3). T1 is SEQ ID NO:7. AP3 is SEQ ID NO:8. Five adenine residues were inserted upstream of AP3 to stabilize the transcribed U6 terminator. FIG. 1B shows pulse-chase experiments measuring the half-life of DS aptamer (NL1F50, T1-50nt-AP3). Cells stably expressing NL1F50 were treated with ActD for the indicated periods. Abundance of NL1F50 was quantified by RT-qPCR and normalized to that of 18S rRNA and initial timepoint (T=0). Data were fitted to a one-phase decay model (curve). N=3. FIGS. 2C and 2D show IP and WB were performed to cells expressing GFP-p-catenin and the indicated aptamers. N.A.—Non-aptamer. endo.—endogenous. One-way ANOVA test (FIG. 2C) or Student's t-test (FIG. 2D) was performed. N=5. FIG. 2E shows WB of total cell lysates from FIG. 2C. FIGS. 2F and 2G show IP and WB of cells expressing GFP-p-catenin and the indicated aptamers and treated with 50 μM Ac5S for 20 hr (FIG. 2F) or 2 μM TMG for 6 hr (FIG. 2G). FIG. 2H is a schematic of the DS aptamer 3JB1F (SEQ ID NO:14) that adopts a three-way RNA junction (PDB 1MFQ) as its linker domain. In this junction, helices P1 and P2 are coaxially stacked and helix P3 is bent towards P1. FIG. 2I is a schematic of the DS aptamer 4JC1 RR (SEQ ID NO:28) that adopts a four-way RNA junction (PDB 1 U9S) as its linker domain. In this junction, helices H3 and H4 are coaxially stacked and helices H1 and H2 are more flexible, meanwhile H2 is attracted to H3. FIG. 2J shows optimization of the folded linker domain in 3JB1F. IP and WB were performed to cells expressing GFP-p-catenin and the indicated aptamers. N=3. FIGS. 2K and 2L show IP and WB (FIG. 2K) or Co-IP between GFP-ERα and OGT (FIG. 2L) of HEK293T cells expressing GFP-ERα and the indicated aptamers. N=3. T1·AP3—Individual T1 and AP3 aptamers (control). NL1F30/50—DS aptamers (T1-linker-AP3) with flexible linkers of 30/50 nt. 3JB1F/4JC1 RR—DS aptamers (T1-linker-AP3) with folded linkers. +2/+4/ . . . /+12, serial additions (bp) into the folded linker. Quantitated WB data are normalized to the control group (T1·AP3). Aptamers and proteins were expressed from plasmids, and One-way ANOVA test was performed unless indicated. Data are represented as mean±SD. ns, p≥0.05; *, p<0.05; **, p<0.01; ***, p<0.001.

FIGS. 3A to 3F show O-GlcNAc stabilizes GFP-p-catenin by inhibiting its interaction with β-TrCP. FIG. 3A contains confocal images of Proximity Ligation Assay (PLA) on HEK293T cells. Cells were co-transfected with plasmids encoding GFP-β-catenin and the control aptamers (T1·AP3) or a DS aptamer (3JB8F+12). PLA was performed with antibodies targeting GFP and β-TrCP. FIG. 3B shows quantification of FIG. 3A. PLA puncta were counted and normalized to the number of nuclei in each view. N=10. Student's t-test was performed. FIGS. 3C to 3F show WB of HEK293T cells co-transfected with the indicated plasmids. In FIG. 3E, cells were treated with 50 μM Ac5S for 20 hr. FIGS. 3D and 3F show quantification of FIGS. 3C and 3E. Intensity of each band was normalized to that of β-tubulin. Np—non-phosphorylated. p—phosphorylated. N=3. T1·AP3—Individual T1 and AP3 aptamers (control). NL1F30—DS aptamers (T1-linker-AP3) with a flexible linker of 30 nt. Quantitated data are normalized to the control group (T1·AP3). Student's t-test was performed. Data are represented as mean±SD. ns, p≥0.05; *, p<0.05; **, p<0.01; ***, p<0.001.

FIGS. 4A to 4O show dual-specificity aptamers increase O-GlcNAcylation on endogenous β-catenin. FIG. 4A shows predicted secondary (left, SEQ ID NO:10) and tertiary (right) structures of bc339. FIG. 4B shows SPR characterization of binding affinity and kinetics of bc339. Sensorgrams represent β-catenin concentrations from 1 μM to 122 μM, in 2× serial dilutions. Data were fitted to a 1:1 binding model. FIG. 4C is a schematic of DS aptamers with flexible linkers (NL8F, T1-linker-bc339). T1 is SEQ ID NO:7·bc339 is SEQ ID NO:10. Five adenine residues were inserted upstream of bc339 to stabilize the transcribed U6 terminator. FIG. 4D shows IP and WB of HEK293T cells expressing the indicated aptamers. N=3. FIGS. 4E and 4F show IP and WB of HEK293T cells expressing the indicated aptamers and treated with 50 μM Ac5S for 20 hr (FIG. 4E) or Wnt3A-conditioned medium for 4 hr (FIG. 4F). Student's t-test was performed. N=3. FIG. 4G is a schematic of DS aptamers (3JB8F, T1-linker-bc339) with folded linkers (SEQ ID NOs:37, 38, 39). FIG. 4H shows predicted tertiary structure of the DS aptamer 3JB8F+12. Nucleotides are color coded in the same way as in FIG. 4H. Complete sequence and secondary structure prediction are shown in FIG. 8C. FIGS. 4I to 4L show IP and WB of HEK293T cells expressing the indicated aptamers, under −Wnt (FIGS. 4I, 4J) and +Wnt (FIGS. 4K, 4L) conditions. FIGS. 4J and 4L show quantification of FIGS. 4I and 4K. N=4. FIGS. 4M and 4N contain confocal images of PLA on HEK293T cells expressing the indicated aptamers. FIG. 4N shows quantification of FIG. 4M. Total PLA puncta was counted and normalized to the number of nuclei in each view. Student's t-test was performed. N=8. FIG. 4O shows subcellular localization of 3JB8F+12. Nucleus and cytoplasm of cells stably expressing 3JB8F+12 was isolated, and RNA in each fraction was quantified by RT-qPCR. RNA abundances were normalized to that of 3JB8F+12 in whole cell lysate (purple bar in upper panel). NEAT1 is a nuclear RNA control, and GAPDH is a cytoplasmic RNA control. Cyt.—Cytoplasm. Nuc.—Nucleus. W.C.—Whole Cell. N=4. T1·bc339—Individual T1 and bc339 aptamers (control). NL8F50/70/100—DS aptamers (T1-linker-bc339) with flexible linkers of 50/70/100 nt. 3JB8F—DS aptamers (T1-linker-bc339) with folded linkers. +4/+12, serial additions (bp) into the folded linker. Quantitated data are normalized to the control group (T1·bc339). Aptamers were expressed from plasmids, and One-way ANOVA test was performed unless indicated. Data are represented as mean±SD. ns, p≥0.05; *, p<0.05; **, p<0.01; ***, p<0.001; ****, p<0.0001.

FIGS. 5A to 5H show O-GlcNAc regulates β-catenin's interaction with EZH2. FIGS. 5A to 5H show confocal images of PLA on HEK293T cells. Cells were transfected with plasmids encoding the control aptamers (T1·bc339) or a DS aptamer (3JB8F+12 or 3JB8R+12) and stimulated with regular (−Wnt) or Wnt3A-conditioned (+Wnt) medium. PLA was performed with antibodies targeting β-catenin and EZH2 or H3K27me3. In FIGS. 5C and 5D, cells were treated with 50 μM Ac5S for 20 hr. FIGS. 5I to 5P show quantification of FIGS. 5A to 5H. PLA puncta in nucleus were counted and normalized to the number of nuclei in each view. N=10 (FIGS. 5I to 5N) or 8 (FIGS. 5O and 5P). Student's t-test was performed. Data are represented as mean±SD. ns, p≥0.05; **, p<0.01; ***, p<0.001; ****, p<0.0001. T1·bc339—Individual T1 and bc339 aptamers (control). 3JB8F+12/R+12—DS aptamers (T1-linker-bc339) with folded linkers.

FIGS. 6A to 6I show O-GlcNAc on β-catenin recruits EZH2 to promoters and shifts the transcriptome. FIGS. 6A and 6B contain Heatmaps of the differential binding sites of EZH2. CUT&RUN sequencing was performed to HEK293T cells transfected with the indicated plasmids and exposed to regular (−Wnt) or Wnt3A-conditioned (+Wnt) medium. FIG. 6C shows the binding peaks of EZH2 on three promoters. FIGS. 6D to 6I contain Volcano plots of RNA-seq data. Cells were transfected with plasmids encoding the control (T1·bc339) or DS (3JB8F+12) aptamers and treated with regular (−Wnt) or Wnt3A-conditioned (+Wnt) medium. In FIGS. 6F and 6G, EZH2 was knocked-down with shRNA. In (H) and (I), cells were treated with Ac5S for 20 hr·T1·bc339—Individual T1 and bc339 aptamers (control). 3JB8F+12—DS aptamer (T1-linker-bc339) with a folded linker.

FIGS. 7A to 7H show inducible regulation of O-GlcNAcylation by coupling dual-specificity aptamers to riboswitches or an inducible expression system. FIGS. 7A and 7B contain schematics of an inducible DS aptamer (LRS1F50C, SEQ ID NO:35) and its conformational transition mechanism. The transmitter elements are in red boxes, their alternative binding sequences are in blue and purple boxes. FIG. 7A shows the “ON” state of LRS1F50C. In the absence of TO1, T1 and AP3 are functionally folded, Mango aptamers are misfolded. FIG. 7B shows the “OFF” state of LRS1F50C. In the presence of TO1, folding of the Mango aptamers disrupts T1 and AP3. FIGS. 7C to 7E show IP and WB performed on cells co-transfected with plasmids encoding GFP-β-catenin and the indicated aptamers, and treated with 750 μM TO1 (+TO1) or equal volume of DMF (−TO1). FIGS. 7D and 7E show quantification of FIG. 7C. N=3. FIG. 7F is a schematic of controlling 3JB8F+12 with a Tet-On system. The DS aptamer 3JB8F+12 is constitutively expressed driven by a wild-type U6 promoter (U6WT). An antidote RNA of T1 (atdT1) is conditionally expressed driven by a Tet-inducible U6 promoter (U6TO), which is controlled by Tet Repressor (TetR) protein and doxycycline (Dox). FIGS. 7G and 7H show IP and WB on cells expressing the indicated aptamers, and treated with Dox (+Dox) or equal volume of DMSO (−Dox). FIG. 7H shows quantification of FIG. 7G. N=3. Two-way ANOVA test was performed. Data are represented as mean±SD. ns, p 0.05; *, p<0.05; ***, p<0.001; ****, p<0.0001.

FIGS. 8A to 8D show predicted secondary structures of 3JB1F+6 (SEQ ID NO:24), 4JC1RR (SEQ ID NO:28), 3JB8F+12 (SEQ ID NO:33), and 3JB8R+12 (SEQ ID NO:24) Dual-specificity aptamers.

FIGS. 9A to 9M show dual-specificity aptamers increase O-GlcNAcylation on GFP-tagged proteins. FIG. 9A shows IP and WB of HEK293T cells co-transfected with plasmids encoding GFP-β-catenin and the indicated aptamers. N=5. FIG. 9B shows quantification of FIG. 9A. Student's t-test was performed. FIG. 9C shows optimization of co-transfection of GFP-β-catenin. IP and WB of cells co-transfected with various amounts of plasmids encoding GFP-β-catenin, and 1 μg plasmids encoding the indicated aptamers. N=3. FIG. 9D shows quantification of FIG. 9C. Data are normalized to the control group (T1·AP3) with 0.1 μg GFP-β-catenin plasmid. Two-way ANOVA test was performed. FIG. 9E shows IP and WB of cells co-transfected with plasmids encoding GFP and the indicated aptamers. The lane with protein ladder is included as a positive control. N=3. FIGS. 9F and 9G show mass-shift assays on cells co-transfected with plasmids encoding GFP-β-catenin and the indicated aptamers. O-GlcNAc on cell lysate was labeled with GalNAz by the enzyme GalT(Y289L), then GalNAz was conjugated to DBCO-PEG (8.5 kDa) in a Click-chemistry reaction. Labeling of O-GlcNAc caused a gel mobility shift of ˜8.5 kDa that was revealed in WB. Two blots with short and long exposure time are shown. Intensity of the shifted bands was normalized to that of β-tubulin. Student's t-test was performed. N=3. FIG. 9H shows IP and WB of cells co-transfected with the indicated plasmids and stimulated with Wnt3A-conditioned medium for 4 hr. N=3. FIG. 9I is a schematic of 3JB1R (SEQ ID NO:21). FIG. 9J shows optimization of the linker domain of 3JB1R. IP and WB of cells co-transfected with plasmids encoding GFP-β-catenin and the indicated aptamers. N=3. FIGS. 9K and 9L show IP and WB (FIG. 9K) or Co-IP between GFP-Src and OGT (FIG. 9L) on cells expressing GFP-Src and the indicated aptamers. N=3. FIG. 9M shows IP and WB of cells expressing GFP-AMPKα2 and the indicated aptamers. N=3. T1·AP3—Individual T1 and AP3 aptamers (control). NL1F35/50—DS aptamers (T1-linker-AP3) with flexible linkers of 35/50 nt. 3JB1F/3JB1R/4JC1 RR—DS aptamers (T1-linker-AP3) with folded linkers. +2/+4/ . . . /+12, serial additions (bp) into the folded linker. Quantitated data are normalized to the control group (T1·AP3). One-way ANOVA test was performed unless indicated. Data are represented as mean±SD. ns, p≥0.05; *, p<0.05; **, p<0.01; ***, p<0.001; ****, p<0.0001.

FIGS. 10A to 10L show novel O-GlcNAc sites are suggested on β-catenin, and O-GlcNAc stabilizes GFP-β-catenin by inhibiting its interaction with β-TrCP. FIGS. 10A, 10C, 10E, and 10G show IP-WB on cells co-transfected with plasmids encoding GFP-β-catenin and the indicated aptamers. In FIGS. 10C and 10G, cells were treated with 50 M Ac5S for 20 hr. N=3. FIGS. 10B, 10D, 10F, and 10H show WB on cells co-transfected with plasmids encoding GFP-β-catenin and the indicated aptamers. In FIGS. 10D and 10H, cells were treated with 50 M Ac5S for 20 hr. In FIGS. 10F and 10H, both chemiluminescence and fluorescence WB were performed and they generated consistent results. Intensity of each band was normalized to that of β-tubulin. N=3. FIG. 10I shows O-GlcNAc on the GFP-β-catenin (S23A, T40A, T41A, T112A) (4A) mutant was reduced by CpOGA. Lysate of HEK293T cells transfected with plasmids encoding GFP-β-catenin (4A) was treated with CpOGA and subjected to IP-WB. N=3. FIG. 10J shows antibody recognition of O-GlcNAc on GFP-β-catenin (4A) was competed by 1 M GlcNAc. IP-WB of cells expressing GFP-β-catenin (4A). 1 M GlcNAc was included in the antibody solution during WB of the membrane on the left. Membranes on the left and right were imaged together for equal exposure. N=3. FIGS. 10K and 10L show quantification of phosphorylation on endogenous β-catenin and abundance of proteins in FIGS. 3D (FIG. 10K) and 3F (FIG. 10L). Intensity of each band was normalized to that of β-tubulin. T1·AP3—Individual T1 and AP3 aptamers (control). NL1F30—DS aptamers (T1-linker-AP3) with flexible linkers of 30 nt. 3JB1F+6—DS aptamers (T1-linker-bc339) with a folded linker. Quantitated data are normalized to the control group (T1·AP3) unless indicated. Student's t-test was performed. Data are represented as mean±SD. ns, p≥0.05; *, p<0.05; **, p<0.01; ***, p<0.001.

FIGS. 11A to 11N show DS aptamers increase O-GlcNAcylation on endogenous β-catenin. FIG. 11A shows WB of whole cell lysate from FIG. 4D and quantification. Intensity of each band in WB was normalized to that of β-tubulin. One-way ANOVA test was performed. N=3. FIG. 11B shows mass-shift assays on HEK293T cells transfected with plasmids encoding the indicated aptamers. Two blots with short and long exposure time are shown. Intensity of the shifted bands was normalized to that of β-tubulin. N=3. FIGS. 11C and 11D show WB of cells transfected with plasmids encoding the indicated aptamers, and treated with regular (FIG. 11C) or Wnt3A-conditioned (FIG. 11D) medium. Intensity of each β-catenin band was normalized to that of β-tubulin. N=3. FIGS. 11E and 11F show WB of total lysate of HEK293T cells transfected with plasmids encoding the indicated aptamers, and treated with regular (FIG. 11E) or Wnt3A-conditioned (FIG. 11F) medium. N=4. FIGS. 11G and 11H show IP and WB of HEK293T cells. Cells were transfected with plasmids encoding the indicated aptamers, treated with Ac5S and regular (FIG. 11G) or Wnt3A-conditioned (FIG. 11H) medium. N=3. FIG. 11I shows co-IP between β-catenin and OGT, on cells transfected with plasmids encoding the indicated aptamers. Intensity of each OGT band was normalized to that of β-catenin. N=3. FIG. 11J shows predicted tertiary structure of the DS aptamer 3JB8R+12. Nucleotides are color coded in the same way as its secondary structure prediction in FIG. 8D. FIGS. 11K and 11L show IP and WB of HEK293T cells transfected with plasmids encoding the indicated aptamers, and treated with regular (FIG. 11K) or Wnt3A-conditioned (FIG. 11L) medium. N=3. FIGS. 11M and 11N show co-IP between β-catenin and EZH2, on cells transfected with plasmids encoding the indicated aptamers, and treated with regular (FIG. 11M) or Wnt3A-conditioned (FIG. 11N) medium. Intensity of each EZH2 band was normalized to that of β-catenin. N=6. T1·bc339—Individual T1 and bc339 aptamers (control). NL8F50/70/100—DS aptamers (T1-linker-bc339) with flexible linkers of 50/70/100 nt. 3JB8F/3JB8R—DS aptamers (T1-linker-bc339) with folded linkers. +12, 12 bp additions into the folded linker. Quantitated data are normalized to the control group (T1·bc339). Student's t-test was performed unless indicated. Data are represented as mean±SD. ns, p≥0.05; *, p<0.05, ***, p<0.001.

FIGS. 12A to 12P show O-GlcNAc regulates β-catenin's interactions with KAT2A. FIGS. 12A to 12H contain confocal images of Proximity Ligation Assay (PLA) on HEK293T cells. Cells were transfected with plasmids encoding the control aptamers (T1·bc339) or a DS aptamer (3JB8F+12 or 3JB8R+12) and stimulated with regular (−Wnt) or Wnt3A-conditioned (+Wnt) medium. PLA was performed with antibodies targeting p3-catenin and KAT2A or H3K9ac. In FIGS. 12C and 12D, cells were treated with 50 μM Ac5S for 20 hr. FIGS. 12I to 12P show quantification of FIGS. 12A to 12H. PLA puncta in nucleus were counted and normalized to the number of nuclei in each view. Student's t-test was performed. Data are represented as mean±SD. ns, p≥0.05; ***, p<0.001; ****, p<0.0001. N=10. T1·bc339—Individual T1 and bc339 aptamers (control). 3JB8F+12/R+12—DS aptamers (T1-linker-bc339) with folded linkers.

FIGS. 13A to 13P show O-GlcNAc regulates β-catenin's interactions with other epigenetic modifiers and β-TrCP. FIGS. 13A to 13F contain confocal images of PLA on HEK293T cells. Cells were transfected with plasmids encoding the control aptamers (T1·bc339) or a DS aptamer (3JB8F+12), and stimulated with regular (−Wnt) or Wnt3A-conditioned (+Wnt) medium. PLA was performed with antibodies targeting β-catenin and EP300, KAT5, or β-TrCP. FIGS. 13G and 13H contain confocal images of the negative control experiments of PLA. Cells were treated with regular (−Wnt) or Wnt3A-conditioned (+Wnt) medium. PLA was performed with the antibody targeting β-catenin and normal rabbit IgG. FIGS. 13I to 13P show quantification of FIGS. 13A to 13H. Nuclear or total PLA puncta were counted and normalized to the number of nuclei in each view. N=10 (I to N) or N=5 (O to P). Student's t-test was performed. Data are represented as mean±SD. **, p<0.01; ***, p<0.001. T1·bc339—Individual T1 and bc339 aptamers (control). 3JB8F+12—A DS aptamer (T1-linker-bc339) with a folded linker.

FIGS. 14A to 14K show O-GlcNAc on β-catenin increases its transcriptional activity and recruits EZH2 to promoters. FIGS. 14A and 14B show distribution of the differential binding sites of EZH2 on the genome, under Wnt− (FIG. 14A) and Wnt+(FIG. 14B) conditions. FIGS. 14C and 14D show the binding peaks of EZH2 on the three promoters shown in FIG. 6C. Cells were knocked-down of β-catenin in FIG. 14C, or treated with Ac5S in FIG. 14D. Cells were transfected with plasmids encoding the control aptamers (T1·bc339) or a DS aptamer (3JB8F+12), and stimulated with regular (−Wnt) or Wnt3A-conditioned (+Wnt) medium. N=3. FIG. 14E shows numbers of the binding peaks of EZH2 on the genome. Analysis was performed on the data shown in FIGS. 6C and S6C. N=3. FIG. 14F shows WB of HEK293T cells that are WT, or stably expressing a shRNA targeting β-catenin or EZH2. Intensity of each band was normalized to that of β-tubulin. N=2. FIGS. 14G to 14H show TOPFlash luciferase reporter assay on HEK293T cells transfected with plasmids encoding the control aptamers (T1·bc339) or a DS aptamer (3JB8F+12), and stimulated with regular (−Wnt) or Wnt3A-conditioned (+Wnt) medium. In (H), cells were treated with 50 μM Ac5S for 20 hr. N=3. FIG. 14I shows TOPFlash assay on cells expressing a scrambled RNA (scrambled), a shRNA targeting OGT (shOGT), or a shRNA targeting OGT with OGT rescued (shOGT+OGT). N=6. FIG. 14J shows TOPFlash assay on cells treated with vehicle (DMSO), an inhibitor of OGT (Ac5S) or an inhibitor of OGA (TMG). N=3. FIG. 14K shows TOPFlash assay on cells transfected with plasmids encoding the indicated RNA. N=3. T1·bc339—Individual T1 and bc339 aptamers (control). 3JB8F+12—A DS aptamer (T1-linker-bc339) with a folded linker. Two-way ANOVA test was performed. Data are represented as mean±SD. ns, p≥0.05; **, p<0.01; ***, p<0.001; ****, p<0.0001.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “specifically binds”, as used herein, when referring to an RNA aptamer, refers to a binding reaction which is determinative of the presence of the protein in a heterogeneous population of proteins and other biologics. Thus, under designated conditions, an RNA aptamer “specifically binds” to its particular “target” when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can include, without limitation, DNA, RNA, cDNA, gDNA, ssDNA, dsDNA or any combination thereof. The term also encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformicacid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “percent (%) sequence identity” or “homology” is defined as the percentage of nucleotides in a candidate sequence that are identical with the nucleotides in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “RNA aptamer” refers to oligonucleotides (e.g. short oligonucleotides), that binds (e.g. with high affinity and specificity) to a target protein. Aptamers can be selected in vitro from very large libraries of randomized sequences by the process of systemic evolution of ligands by exponential enrichment (SELEX). Aptamers may be synthesized by methods which are well known to the skilled person.

The term “riboswitch” refers to synthetic and natural nucleic acid sequences that provide for inducible regulation of the structure (and therefore the availability of specific sequences) of the nucleic acid molecule (e.g., RNA aptamer) of which they are part, RNA aptamers usually comprise a sensor sequence that folds into a particular structure a hairpin), which specifically binds a particular drug. Binding of the drug causes a structural change in the folding of the RNA, which changes a feature of the nucleic acid of which the aptamer is a part. As non-limiting examples: (i) an activator with an aptamer may not be able to bind to the cognate targeter unless the aptamer is bound by the appropriate drug; (ii) a targeter with an aptamer may not be able to bind to the cognate activator unless the aptamer is bound by the appropriate drug; and (iii) a targeter and an activator, each comprising a different aptamer that binds a different drug, may not be able to bind to each other unless both drugs are present.

Examples of aptamers and riboswitches can be found, for example, in: Nakamura et al., Genes Cells, 2012 May; 17(5):344-64; Vavalle et al., Future Cardiol. 2012 May; 8(3):371-82; Citartan et al., Biosens Bioelectron, 2012 Apr. 15; 34(1); 1-11; and Liberman et al., Wiley Interdiscip Rev RNA, 2012 May-June; 3(3):369-84; all of which are herein incorporated by reference in their entirety.

### XXX

### Dual-Specificity RNA Aptamers

Disclosed herein are dual-specificity (DS) aptamers involving modular designed RNA having two aptamer motifs connected by a linker domain that induce proximity between O-GlcNAc transferase (OGT) or O-GlcNAcase (OGA) and a target protein

Therefore, the dual-specificity aptamer contains a first RNA aptamer sequence that targets OGT or OGA and a second RNA aptamer sequence that selectively binds a target protein, wherein the first RNA aptamer sequence and the second RNA aptamer sequence are connected by a linker domain.

The length of a dual-specificity aptamer will reflect the length of the nucleic acid sequence of each mono-specific aptamer incorporated in the dual-specificity aptamer, and the length of any linker that is included. The nucleic acid sequence of an RNA aptamer sequence may optionally have a minimum length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides and a maximum length of 90, 91, 92, 93, 94, 95, 96, 97, 98, 99, or 100 nucleotides.

### RNA Aptamers for OGT

In some embodiments, the first RNA Aptamer sequence that selectively binds OGT has the nucleic acid sequence GGGAGAAUCACUUACGGAUCCUCGUUAAGUGGCUCUCAGUCGCCCGUAACUCCG AUGCCGAUGCAGCGGCGACAACUGCACCCAAGGCUCGGAAGCUU (SEQ ID NO:7, T1), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, 94, 95, 96, 97, or 98 nucleotides in length having at least 90%, 91%, 92%, 93%, 94%, 95%, 96%, 97%, 98%, 99%, or 100% sequence identity to SEQ ID NO:7 that is able to selectively bind OGT.

In some embodiments, the first RNA Aptamer sequence that selectively binds OGT has the nucleic acid sequence GGGAGAAUCACUUACGGAUCCUCCCGUCUUUGGACUGGUCCGACAGGAAAAGCA UUAGAAUGUGCGCCCUGGUUGAUCCAAGGCUCGGAAGCUU (SEQ ID NO:42, OGT1), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, or 94 nucleotides in length having at least 90%, 91%, 92%, 93%, 94%, 95%, 96%, 97%, 98%, 99%, or 100% sequence identity to SEQ ID NO:42 that is able to selectively bind OGT.

In some embodiments, the first RNA Aptamer sequence that selectively binds OGT has the nucleic acid sequence GGGAGAAUCACUUACGGAUCCCGGAAAAAUAAUAACGAGAAGCGAAAAGUAAGUA GAUUACUCCUUUGCUUCGGCCCCAAGGCUCGGAAGCUU (SEQ ID NO:43, OGT2), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:43 that is able to selectively bind OGT.

In some embodiments, the first RNA Aptamer sequence that selectively binds OGT has the nucleic acid sequence GGGAGAAUCACUUACGGAUCCGUUUUGAGACUUGCUCCGAUUCACAAGAAAAAU AAUAAGAAAGUUGGAUCGGAGUCCAAGGCUCGGAAGCUU (SEQ ID NO:44, OGT3), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:44 that is able to selectively bind OGT.

In some embodiments, the first RNA Aptamer sequence that selectively binds OGT has the nucleic acid sequence GGGAGAAUCACUUACGGAUCCUUGAGAAAAAUAAGAAUAAGUGAGUGUCGCGUA AGAUGGUAAAUCCCUUACUCUCCCAAGGCUCGGAAGCUU (SEQ ID NO:45, OGT4), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:45 that is able to selectively bind OGT.

In some embodiments, the first RNA Aptamer sequence that selectively binds OGT has the nucleic acid sequence GGGAGAAUCACUUACGGAUCCUUCUGCCGAGUUAUUGGUGACGACCUUAAAAGA AUAAUAACAAACGUGUCGUUCGCCAAGGCUCGGAAGCUU (SEQ ID NO:46, OGT7), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:46 that is able to selectively bind OGT.

In some embodiments, the first RNA Aptamer sequence that selectively binds OGT has the nucleic acid sequence GGGAGAAUCACUUACGGAUCCCGGAUCUUUCGAUUUCAGCCGAAGCCUUGAUAA UAAUAAAGAUAACUGGUUUGACCCAAGGCUCGGAAGCUU (SEQ ID NO:47, OGT8), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:47 that is able to selectively bind OGT.

In some embodiments, the first RNA Aptamer sequence that selectively binds OGT has the nucleic acid sequence GGGAGAAUCACUUACGGAUCCUGGAGAAGACAAAUUAGAAUAGGGCGCCCAGUG ACCUGUAAGUGAAGGGCCUCCUCCAAGGCUCGGAAGCUU (SEQ ID NO:48, OGT9), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:48 that is able to selectively bind OGT.

### RNA Aptamers for OGA

In some embodiments, the first RNA Aptamer sequence that selectively binds OGA has the nucleic acid sequence GGGAGAAUCACUUACGGAUCCCGUUUGAUCUGACGGCCCUUGGAUAGCACGAGA AUUAGAACAAUAAGUGUGUUUUUCCAAGGCUCGGAAGCUU (SEQ ID NO:49, OGA1), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, or 94 nucleotides in length having at least 90%, 91%, 92%, 93%, 94%, 95%, 96%, 97%, 98%, 99%, or 100% sequence identity to SEQ ID NO:49 that is able to selectively bind OGA.

In some embodiments, the first RNA Aptamer sequence that selectively binds OGA has the nucleic acid sequence GGGAGAAUCACUUACGGAUCCAGUUACAAUAAUAAGAACAUAAACUUGGAUUCGU UUGUGAGCUUACCGACUUAUUCCAAGGCUCGGAAGCUU (SEQ ID NO:50, OGA2), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:50 that is able to selectively bind OGA.

In some embodiments, the first RNA Aptamer sequence that selectively binds OGA has the nucleic acid sequence GGGAGAAUCACUUACGGAUCCUUUUAUCGAGUCUAGGAUGCGACAAUCAACAAU AAGAACAAUAAGAUCUGUCGCGCCAAGGCUCGGAAGCUU (SEQ ID NO:51, OGA3), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:51 that is able to selectively bind OGA.

In some embodiments, the first RNA Aptamer sequence that selectively binds OGA has the nucleic acid sequence GGGAGAAUCACUUACGGAUCCUAUGACGUUCAAAAAAUAAGAAUAACAAAGAGCG UCAUGGUUGAUCUGUCCCGGGCCAAGGCUCGGAAGCUU (SEQ ID NO:52, OGA4), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:52 that is able to selectively bind OGA.

In some embodiments, the first RNA Aptamer sequence that selectively binds OGA has the nucleic acid sequence GGGAGAAUCACUUACGGAUCCUUUUCUUCCAAGUCUUCGGACACGACUAUAAUA ACAAUAAGAAAAUGUUGCCCGUCCAAGGCUCGGAAGCUU (SEQ ID NO:53, OGA6), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:53 that is able to selectively bind OGA.

In some embodiments, the first RNA Aptamer sequence that selectively binds OGA has the nucleic acid sequence GGGAGAAUCACUUACGGAUCCUUUCACGAGUCUUCGCUAUGAUAACAAUAACAA UAACGUGUCGGUUGACACCUAGCCAAGGCUCGGAAGCUU (SEQ ID NO:54, HAT1), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:54 that is able to selectively bind OGA.

In some embodiments, the first RNA Aptamer sequence that selectively binds OGA has the nucleic acid sequence GGGAGAAUCACUUACGGAUCCUCUCCCUUGUCCUUGGGAUUUCGGUAACGAUAA UAACAAUAAAAAGGGUUGUCGACCAAGGCUCGGAAGCUU (SEQ ID NO:55, HAT2), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:55 that is able to selectively bind OGA.

In some embodiments, the first RNA Aptamer sequence that selectively binds OGA has the nucleic acid sequence GGGAGAAUCACUUACGGAUCCGACCUCUUUCCUGUCUUGGCGGUAUCGGUACUA UAAUAAUAAAAAGCGCAUAUCGCCAAGGCUCGGAAGCUU (SEQ ID NO:56, HAT12), or a variant and/or fragment thereof at least 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, or 93 nucleotides in length having at least 90%, 91%, 92%, 93%, 94%, 95%, 96%, 97%, 98%, 99%, or 100% sequence identity to SEQ ID NO:56 that is able to selectively bind OGA.

### RNA Aptamers for Target Proteins

In some embodiments, the target protein is any protein that is modified by O-GlcNAc transferase (OGT).

For example, β-catenin is known to be regulated by O-GlcNAcylation. Therefore, in some embodiments, the second RNA Aptamer sequence can selectively bind β-catenin. As an example, the second RNA Aptamer sequence can have the nucleic acid sequence AGCUUCUGGGCGGCGAUGAGAUGACGUGUGCGGUGGUCGAGAGGUACCUUGGG UGAGGGAAGGGAAGGGAGGUUGACCACUGCGUGACUGCCCAGAAGCU (SEQ ID NO:10, bc339), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, or 84 nucleotides in length having at least 90%, 91%, 92%, 93%, 94%, 95%, 96%, 97%, 98%, 99%, or 100% sequence identity to SEQ ID NO:10 that is able to selectively bind β-catenin.

As an example, the second RNA Aptamer sequence can have the nucleic acid sequence GGGCGGCGAUGAGAUGACUCUUUGCGAUAAUAAUAACAAAAAGGGUAUUUGAGC CAACUCGUUCGCCUGACCACUGCGUGACUGCC (SEQ ID NO:40, bc1), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, or 86 nucleotides in length having at least 90%, 91%, 92%, 93%, 94%, 95%, 96%, 97%, 98%, 99%, or 100% sequence identity to SEQ ID NO:40 that is able to selectively bind β-catenin.

As an example, the second RNA Aptamer sequence can have the nucleic acid sequence GGGCGGCGAUGAGAUGACAAAGCGAUAAUAAGAACAAUAAGGGCUUUCUUUCUG UCGUAGCUAUUGGUACCACUGCGUGACUGCC (SEQ ID NO:41, bc2), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, or 85 nucleotides in length having at least 90%, 91%, 92%, 93%, 94%, 95%, 96%, 97%, 98%, 99%, or 100% sequence identity to SEQ ID NO:41 that is able to selectively bind β-catenin.

As another example, p53 is known to be regulated by O-GlcNAcylation. Therefore, in some embodiments, the second RNA Aptamer sequence can selectively bind p53. As an example, the second RNA Aptamer sequence can have the nucleic acid sequence GGGCGAAUUCGGGUUGGAUAGUAGGCGCAUAUGGCAUCUUCGUGGUUGUGUAU UGCCCUUUAGUGAGGGUUAAUU (SEQ ID NO:57), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, or 75 nucleotides in length having at least 90%, 91%, 92%, 93%, 94%, 95%, 96%, 97%, 98%, 99%, or 100% sequence identity to SEQ ID NO:57 that is able to selectively bind p53.

As another example, Hsp70 is known to be regulated by O-GlcNAcylation. Therefore, in some embodiments, the second RNA Aptamer sequence can selectively bind Hsp70. As an example, the second RNA Aptamer sequence can have the nucleic acid sequence GGGCCGAGAAUUCAACUGCCAUCUAGGCCUUAUAAACAGCCGGAUCCCGAUUGU GCUCGAUAUGUACUCGGCCC (SEQ ID NO:58, AptHsp70-1-66S), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, or 75 nucleotides in length having at least 90%, 91%, 92%, 93%, 94%, 95%, 96%, 97%, 98%, 99%, or 100% sequence identity to SEQ ID NO:58 that is able to selectively bind Hsp70.

As another example, CP-1 and CP-2 are derivatives of 66S that are known to be regulated by O-GlcNAcylation. Therefore, in some embodiments, the second RNA Aptamer sequence can selectively bind CP-1 or CP-2. As an example, the second RNA Aptamer sequence can have the nucleic acid sequence GGGCAUUGUGCUCGAUAUGUACUCCUUCGGGAGAAUUCAACUGCCAUCUAGGCC UUAUAAACAGUGCCC (SEQ ID NO:59), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, or 69 nucleotides in length having at least 90%, 91%, 92%, 93%, 94%, 95%, 96%, 97%, 98%, 99%, or 100% sequence identity to SEQ ID NO:59 that is able to selectively bind CP-1. As an example, the second RNA Aptamer sequence can have the nucleic acid sequence GGGAGGCCUUAUAAACAGCCGGAUCCCGAUUGUGCUCGAUAUGUACUCCUUCGG GAGAAUUCAACUGCCUCCC (SEQ ID NO:60), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, or 73 nucleotides in length having at least 90%, 91%, 92%, 93%, 94%, 95%, 96%, 97%, 98%, 99%, or 100% sequence identity to SEQ ID NO:60 that is able to selectively bind CP-2.

As another example, Estrogen Receptor α (ERα) is known to be regulated by O-GlcNAcylation. Therefore, in some embodiments, the second RNA Aptamer sequence can selectively bind ERα. As an example, the second RNA Aptamer sequence can have the nucleic acid sequence GGGAGAAUUCAACUGCCAUCUAGGCCCACAGUUCAGAGGCACCGCGAACAAAAC GCAAGACAGAGUGCCGACAAGAGCACUACAAGCUUCUGGACUCGGU (SEQ ID NO:61, AptER-1), or a variant and/or fragment thereof at least 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, 94, 95, 96, 97, 98, 99, or 100 nucleotides in length having at least 90%, 91%, 92%, 93%, 94%, 95%, 96%, 97%, 98%, 99%, or 100% sequence identity to SEQ ID NO:61 that is able to selectively bind ERα.

As another example, the second RNA Aptamer sequence can have the nucleic acid sequence GGGCAGAGGCACCGCGAACAAAACGCAAGACAGAGUGCCGACAAGAGCACUACA AGCUUCUGCCC (SEQ ID NO:62, AptER-1-65nt), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, or 65 nucleotides in length having at least 90%, 91%, 92%, 93%, 94%, 95%, 96%, 97%, 98%, 99%, or 100% sequence identity to SEQ ID NO:62 that is able to selectively bind ERα.

As another example, the second RNA Aptamer sequence can have the nucleic acid sequence GGGAGAAUUCAACUGCCAUCUAGGCACACGCGAGAUAGAGCGAGGCCUCCAAAA AUGGCCACGCCAGGAAGCAAGUACUACAAGCUUCUGGACUCGGU (SEQ ID NO:63, AptER-2), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, 94, 95, 96, 97, or 98 nucleotides in length having at least 90%, 91%, 92%, 93%, 94%, 95%, 96%, 97%, 98%, 99%, or 100% sequence identity to SEQ ID NO:63 that is able to selectively bind ERα.

As another example, the second RNA Aptamer sequence can have the nucleic acid sequence GGGAGAAUUCAACUGCCAUCUAGGCGACCCAGGGCCGGGACGCAAAGCAGCCAA AACAGACGGCCCCAGUCAGGGAGUUACUACAAGCUUCUGGACUCGGU (SEQ ID NO:64, AptER-3), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, 84, 85, 86, 87, 88, 89, 90, 91, 92, 93, 94, 95, 96, 97, 98, 99, 100, or 101 or nucleotides in length having at least 90%, 91%, 92%, 93%, 94%, 95%, 96%, 97%, 98%, 99%, or 100% sequence identity to SEQ ID NO:64 that is able to selectively bind ERα.

As another example, NF-κB p65 is known to be regulated by O-GlcNAcylation. Therefore, in some embodiments, the second RNA Aptamer sequence can selectively bind NF-κB p65. As an example, the second RNA Aptamer sequence can have the nucleic acid sequence GAAGCUUACAAGAAGGACAGCACGAAUAAAACCUGCGUAAAUCCGCCCCAUUUGU GUAAGGGUAGUGGGUCGAAUUCCGCUCA (SEQ ID NO:65), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, or 83 nucleotides in length having at least 90%, 91%, 92%, 93%, 94%, 95%, 96%, 97%, 98%, 99%, or 100% sequence identity to SEQ ID NO:65 that is able to selectively bind NF-κB p65.

As another example, NF-κB p50 is known to be regulated by O-GlcNAcylation. Therefore, in some embodiments, the second RNA Aptamer sequence can selectively bind NF-κB p50. As an example, the second RNA Aptamer sequence can have the nucleic acid sequence GGAUCCUGAAACUGUUUUAAGGUUGGCCGAUC (SEQ ID NO:66, A-p50-1), or a variant and/or fragment thereof at least 20, 21, 22, 23, 24, 25, 26, 27, 28, 29, 30, 31, or 32 nucleotides in length having at least 90%, 91%, 92%, 93%, 94%, 95%, 96%, 97%, 98%, 99%, or 100% sequence identity to SEQ ID NO:66 that is able to selectively bind NF-κB p50.

As another example, the second RNA Aptamer sequence can have the nucleic acid sequence CAGGCUGCAUGAUGGAACAGCCCAUAACAUACUUGAAACUGUAAGGUUGGCGUA UGCAUGAAGCGUUCCAUGCAUGCCUG (SEQ ID NO:67, A-p50-6), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, or 80 nucleotides in length having at least 90%, 91%, 92%, 93%, 94%, 95%, 96%, 97%, 98%, 99%, or 100% sequence identity to SEQ ID NO:67 that is able to selectively bind NF-κB p50.

As another example, the second RNA Aptamer sequence can have the nucleic acid sequence GGCCGCCCGGCGCCAUACUUGAAACUGUAAGGUUGGCGUAUGGCGCCGGGCGG CC (SEQ ID NO:68, A-p50-7), or a variant and/or fragment thereof at least 40, 41, 42, 43, 44, 45, 46, 47, 48, 49, 50, 51, 52, 53, 54, or 55 nucleotides in length having at least 90%, 91%, 92%, 93%, 94%, 95%, 96%, 97%, 98%, 99%, or 100% sequence identity to SEQ ID NO:68 that is able to selectively bind NF-κB p50.

In some embodiments, the target protein is a tag, such as a His tag, MS2-tag, a fluorochrome, e.g. GFP, YFP, etc. . . .

As an example, the second RNA Aptamer sequence can have the nucleic acid sequence AGCUUCUGGACUGCGAUGGGAGCACGAAACGUCGUGGCGCAAUUGGGUGGGGA AAGUCCUUAAAAGAGGGCCACCACAGAAGCU (SEQ ID NO:8, AP3), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, 80, 81, 82, 83, or 84 nucleotides in length having at least 90%, 91%, 92%, 93%, 94%, 95%, 96%, 97%, 98%, 99%, or 100% sequence identity to SEQ ID NO:8, that is able to selectively bind GFP.

RNA aptamers that selectively bind to polyhistidine-tag are described in Tsuji, et al. Biochemical and Biophysical Research Communications 2009 386:227-231, which is incorporated by reference in its entirety for the teaching of these aptamers. As an example, the second RNA Aptamer sequence can have the nucleic acid sequence GGGACGCUCACGUACGCUCACGUCCGAUCGAUACUGGUAUAUUGGCGCCUUCG UGGAAUGUCAGUGCCUGGACGUGCAGU (SEQ ID NO:69, Shot47), or a variant and/or fragment thereof at least 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, 70, 71, 72, 73, 74, 75, 76, 77. 78, 79, or 80 nucleotides in length having at least 90%, 91%, 92%, 93%, 94%, 95%, 96%, 97%, 98%, 99%, or 100% sequence identity to SEQ ID NO:69 that is able to selectively bind polyhistidine-tag.

### Riboswitch

In some embodiments, the dual-specificity aptamer comprises a ligand-responsive riboswitch comprising a sensor sequence that activates or deactivates the dual-specificity aptamer upon binding of the ligand to the sensor sequence.

A frequently used SELEX-derived aptamer in synthetic riboswitches is the theophylline-binding aptamer TCT8-4. This aptamer has a high discriminatory potential against structurally closely related purines like caffeine, which differs from theophylline only by a single methyl group at position N7. Yet, the discrimination is 10,000-fold. With an aptamer binding constant of 320 mM and good cell permeability, theophylline represents an excellent ligand for synthetic riboswitches, despite its cytotoxicity at higher concentrations. While the ligand-free structure of the aptamer is rather dynamic and adopts several different conformations, binding of theophylline induces a structural rearrangement, stabilizing a highly defined and robust structure.

Another aptamer identified by SELEX successfully used in riboswitch-mediated regulation of translation is the tetracycline-binding aptamer. Similar to the theophylline aptamer, the tetracycline-unbound state of the RNA exists in a less stable pre-formed scaffold, and ligand binding induces a conformational rearrangement resulting in a more compact structure. As a result, the interaction of the RNA with tetracycline shifts the thermodynamic equilibrium from the ligand-free structure towards the tetracycline-bound structure.

The following is a non-limiting list of riboswitches that include aptamers: cobalamin riboswitches (also B12-element), cyclic di-GMP riboswitches, FMN riboswitches (also RFN-element), GlmS riboswitches, glycine riboswitches, lysine riboswitches (also L-box), PreQ1 riboswitches, purine riboswitches, SAH riboswitches, SAM riboswitches, tetrahydrofolate riboswitches, theophylline riboswitches, and TPP riboswitches (also THI-box).

Cobalamin riboswitches (also B12-element) refer to riboswitches that bind adenosylcobalamin (the coenzyme form of vitamin B12) to regulate cobalamin biosynthesis and transport of cobalamin and similar metabolites, and other genes. See, e.g., Nahvi et al., “Coenzyme B12 riboswitches are widespread genetic control elements in prokaryotes.” Nucleic Acids Res. 2004; 32: 143-150; Vitreschak et al., “Regulation of the vitamin B12 metabolism and transport in bacteria by a conserved RNA structural element.” RNA. 2003; 9:1084-1097; the entire contents of each are hereby incorporated by reference.

### Compositions

Also disclosed are pharmaceutical compositions of the disclosed RNA aptamers in a pharmaceutically acceptable excipient. “Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Specific Embodiments

Embodiment 1. A dual-specificity aptamer comprising a first RNA aptamer sequence that targets O-GlcNAc transferase (OGT) or O-GlcNAcase (OGA) and a second RNA aptamer sequence that selectively binds a target protein, wherein the first RNA aptamer sequence and the second RNA aptamer sequence are connected by a linker region.

Embodiment 2. The dual-specificity aptamer of embodiment 1, wherein the first RNA aptamer targets O-GlcNAc transferase (OGT).

Embodiment 3. The dual-specificity aptamer of embodiment 2, wherein the first RNA aptamer targets the nucleocytoplasmic isoform of OGT.

Embodiment 4. The dual-specificity aptamer of embodiment 3, wherein the first RNA aptamer comprises the nucleic acid sequence SEQ ID NO:7, 42, 43, 44, 45, 46, 47, or 48, or a variant and/or fragment thereof at least 90 nucleotides in length having at least 90% sequence identity to SEQ ID NO: 42, 43, 44, 45, 46, 47, or 48 that is able to bind OGT.

Embodiment 5. The dual-specificity aptamer of embodiment 1, wherein the first RNA aptamer targets O-GlcNAcase (OGA) as the first protein.

Embodiment 6. The dual-specificity aptamer of embodiment 5, wherein the first RNA aptamer comprises the nucleic acid sequence SEQ ID NO:49, 50, 51, 52, 53, 54, 55, or 56, or a variant and/or fragment thereof at least 90 nucleotides in length having at least 90% sequence identity to SEQ ID NO:49, 50, 51, 52, 53, 54, 55, or 56 that is able to bind OGA.

Embodiment 7. The dual-specificity aptamer of any one of embodiments 1 to 6, wherein the target protein is β-catenin.

Embodiment 8. The dual-specificity aptamer of embodiment 7, wherein the second RNA aptamer comprises the nucleic acid sequence SEQ ID NO:10, or a variant and/or fragment thereof at least 74 nucleotides in length having at least 90% sequence identity to SEQ ID NO:10 that is able to selectively bind β-catenin.

Embodiment 9. The dual-specificity aptamer of any one of embodiments 1 to 6, wherein the target protein is green fluorescent protein (GFP).

Embodiment 10. The dual-specificity aptamer of embodiment 9, wherein the second RNA aptamer comprises the nucleic acid sequence SEQ ID NO:8, or a variant and/or fragment thereof at least 74 nucleotides in length having at least 90% sequence identity to SEQ ID NO:8, that is able to selectively bind GFP.

Embodiment 11. The dual-specificity aptamer of any one of embodiments 1 to 10, wherein the second RNA aptamer sequence is generated for a target protein using Systematic Evolution of Ligands by EXponential enrichment.

Embodiment 12. The dual-specificity aptamer of any one of embodiments 1 to 11, wherein the linker region is selected from a flexible linker and a folded linker.

Embodiment 13. The dual-specificity aptamer of embodiment 12, wherein the flexible linker is from 10 to 100 nucleotides in length.

Embodiment 14. The dual-specificity aptamer of embodiment 12, wherein the linker comprises three-way RNA junctions or four-way RNA junctions.

Embodiment 15. The dual-specificity aptamer of any one of embodiments 1 to 14, wherein the dual-specificity aptamer comprises a ligand-responsive riboswitch comprising a sensor sequence that activates or deactivates the dual-specificity aptamer upon binding of the ligand to the sensor sequence.

Embodiment 16. The dual-specificity aptamer of embodiment 15, wherein the ligand-responsive riboswitch is selected from the group consisting of a theophylline riboswitch, a thiamine pyrophosphate (TPP) riboswitch, an adenosine cobalamin (AdoCbl) riboswitch, an S-adenosyl methionine (SAM) riboswitch, an SAH riboswitch, a flavin mononucleotide (FMN) riboswitch, a tetrahydrofolate riboswitch, a lysine riboswitch, a glycine riboswitch, a purine riboswitch, a guanine riboswitch, a GlmS riboswitch, or a pre-queosine1 (PreQ1) riboswitch.

Embodiment 17. A vector comprising a nucleic acid sequence encoding the dual dual-specificity aptamer of any one of embodiments 1 to 16 operably linked to an expression control system.

Embodiment 18. The vector of embodiment 17, wherein the expression control system comprises an inducible promoter or a tissue specific promoter.

Embodiment 19. A composition comprising the dual-specificity aptamer of any one of embodiments 1 to 16 in a pharmaceutically acceptable carrier.

Embodiment 20. A method of modulating O-GlcNAc on a protein, comprising contacting the protein with the composition of embodiment 19.

Embodiment 21. An RNA aptamer comprising the nucleic acid sequence SEQ ID NO:7, or a variant and/or fragment thereof at least 90 nucleotides in length having at least 90% sequence identity to SEQ ID NO:7 that is able to bind O-GlcNAc transferase (OGT).

Embodiment 22. An RNA aptamer comprises the nucleic acid sequence SEQ ID NO:49, 50, 51, 52, 53, 54, 55, or 56, or a variant and/or fragment thereof at least 90 nucleotides in length having at least 90% sequence identity to SEQ ID NO:49, 50, 51, 52, 53, 54, 55, or 56 that is able to bind OGA.

Embodiment 23. An RNA aptamer comprises the nucleic acid sequence SEQ ID NO:10, 40, or 41, or a variant and/or fragment thereof at least 74 nucleotides in length having at least 90% sequence identity to SEQ ID NO:10, 40, or 41 that is able to selectively bind β-catenin.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Aptamers Increase O-GlcNAc on a Single Protein to Regulate Wnt Signaling

### Results

A Noninhibiting RNA Aptamer Targeting ncOGT was Generated from SELEX

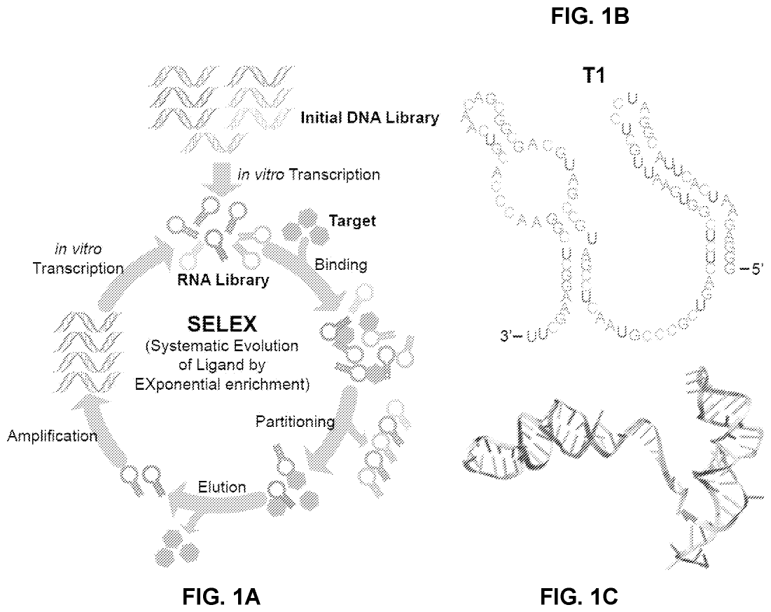

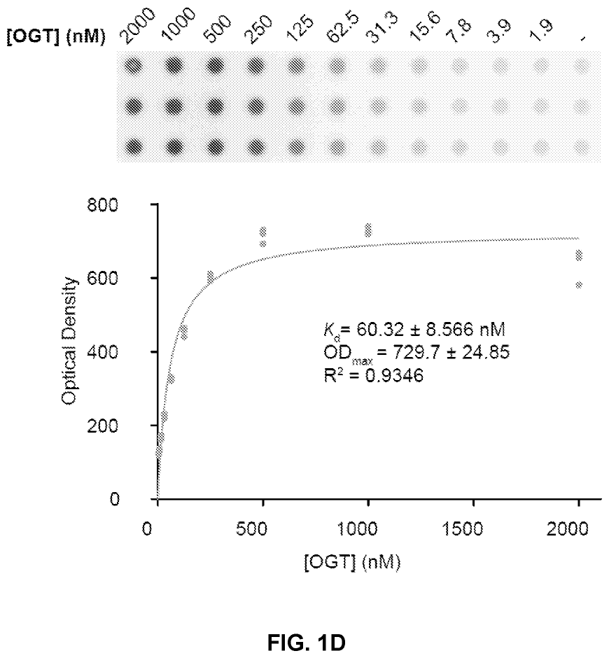

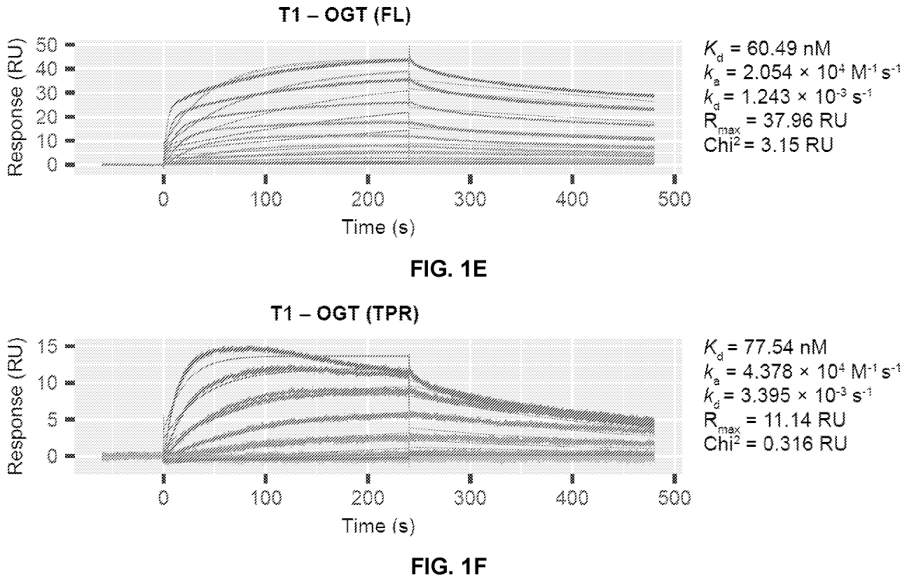

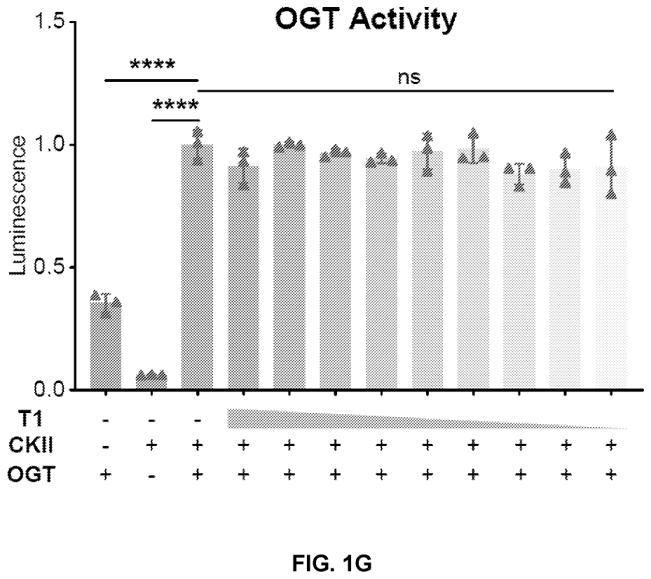

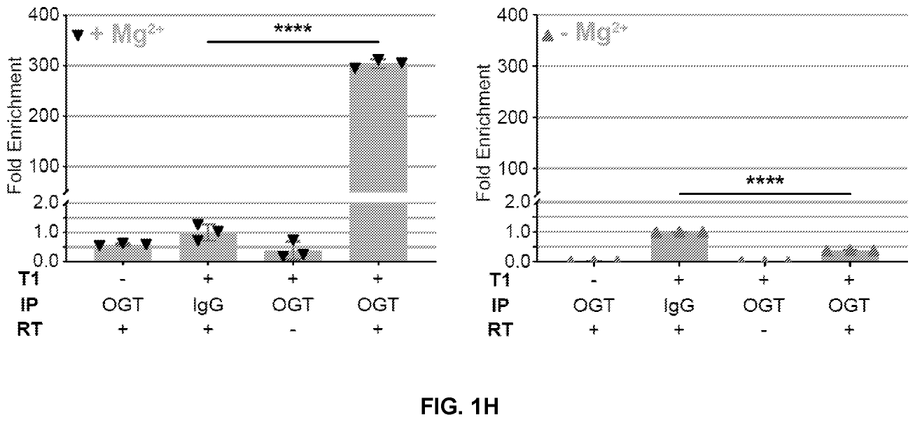

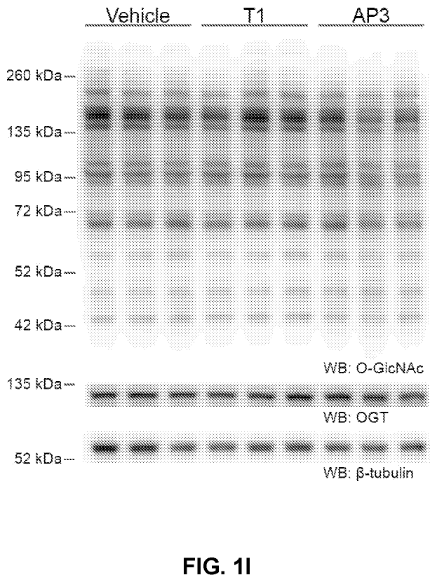

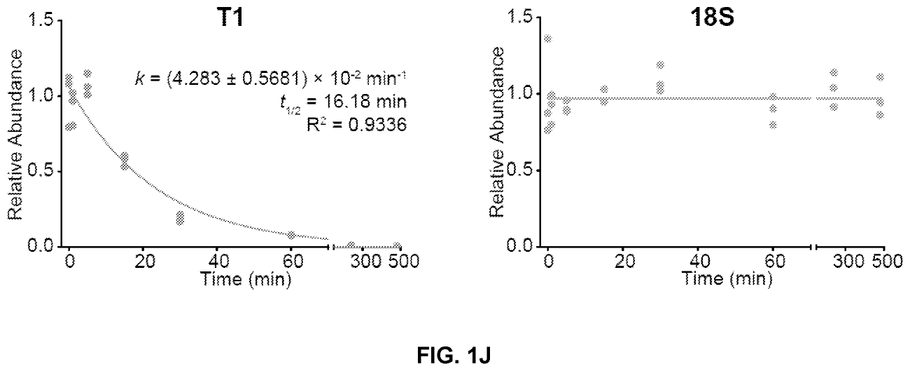

Discovered in 1990, aptamers are short, single-stranded DNA or RNA molecules that bind targets with high affinities, and specificities comparable to those of monoclonal antibodies. They have been selected to bind a wide variety of targets from metal ions, small organic molecules, proteins to whole cells (Ellington, A. D., et al. Nature 1990 346:818-822; Tuerk, C., et al. Science 1990 249:505-510; Lakhin, A. V., et al. Acta Naturae 2013 5:34-43; Tan, Y., et al. Anal Chem 2014 86:9466-9472). Aptamers are generated from the in vitro selection named Systematic Evolution of Ligands by EXponential enrichment (SELEX, FIG. 1A), which is an iterative binding, partitioning and amplification process (Bunka, D. H., et al. Nat Rev Microbiol 2006 4:588-596).

To regulate O-GlcNAcylation with DS aptamers, it is a prerequisite that we have a noninhibiting RNA aptamer targeting the nucleocytoplasmic isoform of OGT (ncOGT). Therefore, SELEX was performed. To generate aptamers that bind the non-catalytic domain of OGT, the targets of SELEX were switched between the full-length ncOGT and its tetratricopeptide repeat (TPR) domain (Table 2) (Lazarus, M. B., et al. Nature 2011 469:564-567). After 11 rounds of SELEX, the sequence T1 was highly enriched in the final RNA library (FIGS. 1B and 1C; Table 1).

T1 was validated as a noninhibiting aptamer targeting ncOGT in vitro. Binding between T1 and ncOGT was confirmed in Radiolabeled Dot-Blot Assays (FIG. 1D), and further characterized in Surface Plasmon Resonance (SPR) (FIG. 1E). In both experiments, the dissociation constant (K_{d}) was measured to be −60 nM. SPR also shows that T1 recognizes the TPR domain of ncOGT (FIG. 1F). To study if T1 inhibits the activity of the enzyme, UDP-Glo OGT activity assays were performed (FIG. 1G). No inhibition was observed when T1 was included in the reactions from 7.8 nM to 2 μM, which was approximately 33 times higher than the K_{d}.

T1 was further validated in living cells. The binding between T1 and OGT in HEK293T cells was confirmed in RNA-IP experiments (FIG. 1H, left). When expressed in cells, T1 was highly enriched on immunoprecipitated OGT protein. This binding requires Mg²⁺. In RNA-IP reactions containing no Mg²⁺, T1 was no longer enriched on OGT (FIG. 1H, right). The divalent Mg²⁺ ion facilitates RNA folding by shielding the negative charges on the RNA backbone (Pyle, A. M. J Biol Inorg Chem 2002 7:679-690; Carothers, J. M., et al. Nucleic Acids Res 2010 38:2736-2747), and Mg²⁺ was included in the selection buffer in SELEX. In cells, expression of T1 did not change the global O-GlcNAcylation pattern or the abundance of OGT (FIG. 1I). Moreover, in pulse-chase experiments with actinomycin D (ActD) treatment, T1 was found to have a short half-life (16.18 min) in cells (FIG. 1J, left). In contrast, 18S rRNA was stable during the 8-hr treatment (FIG. 1J, right).

In conclusion, T1 is an RNA aptamer that binds OGT without inhibiting its enzymatic activity both in vitro and in cells. It is a short-lived RNA in cellular context.

### Dual-Specificity Aptamers Increase O-GlcNAcylation on GFP-Tagged Proteins

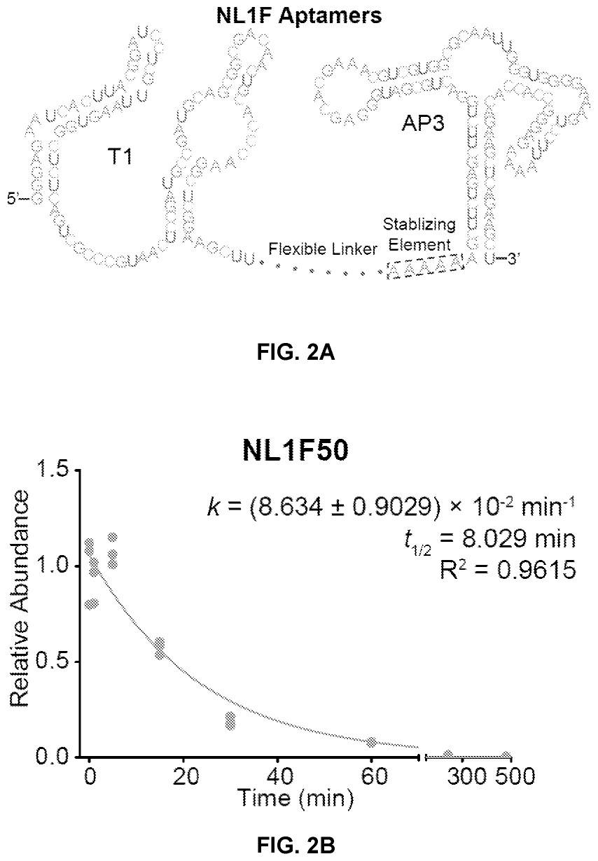

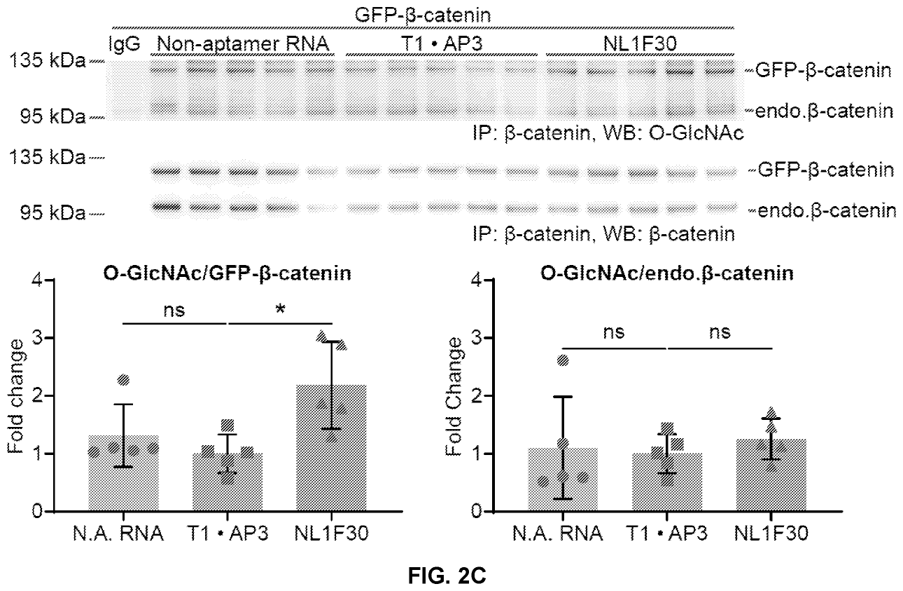

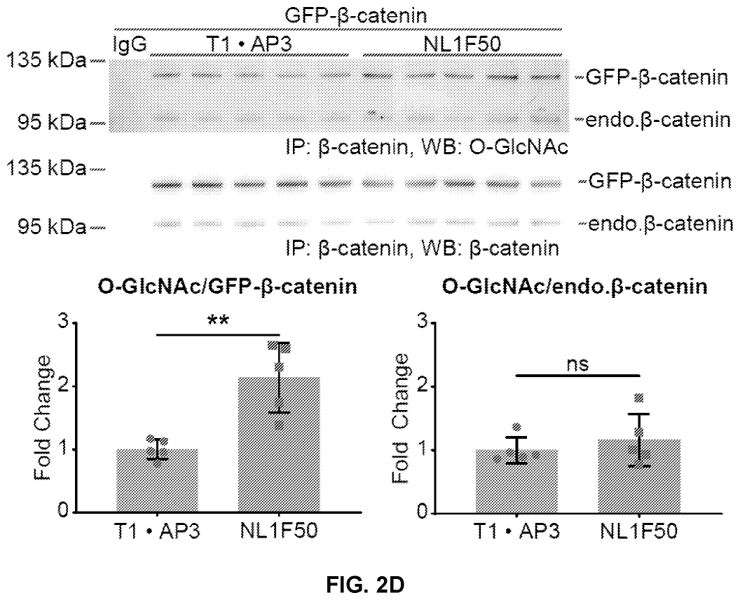

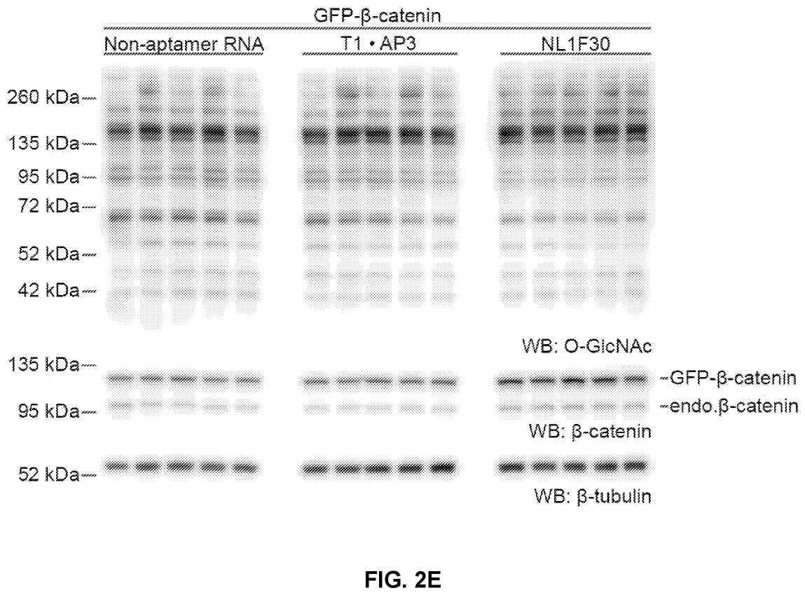

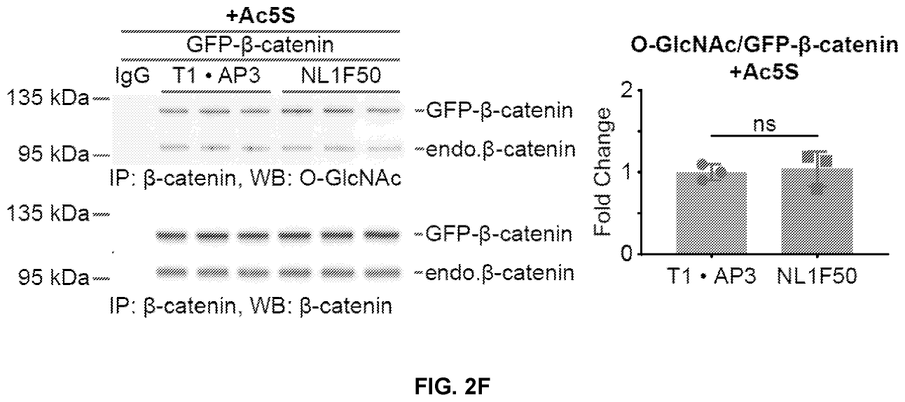

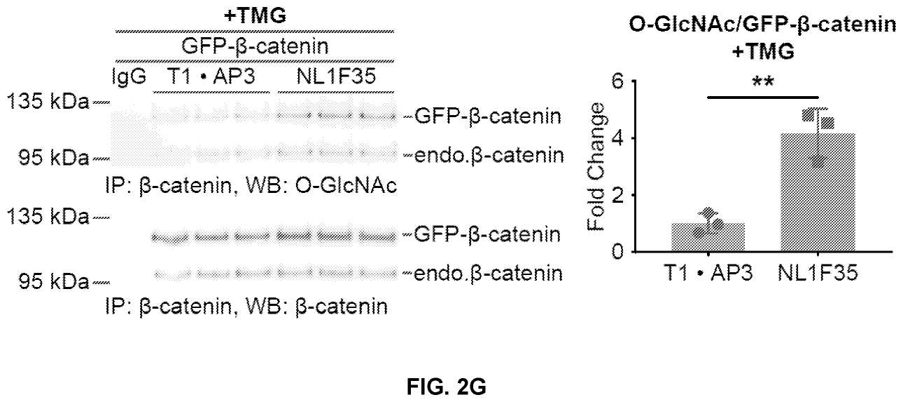

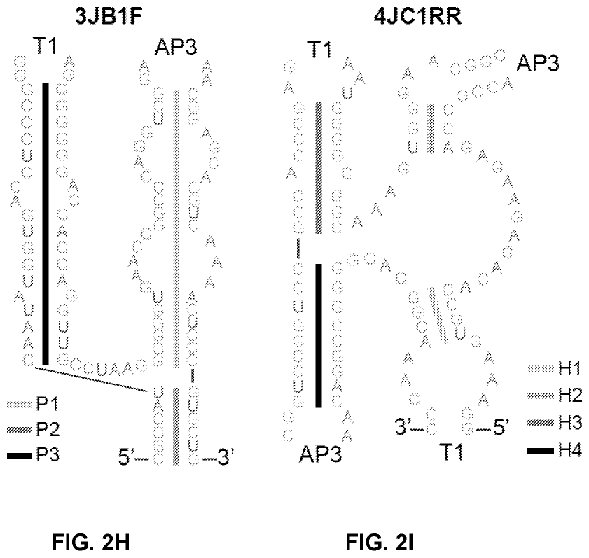

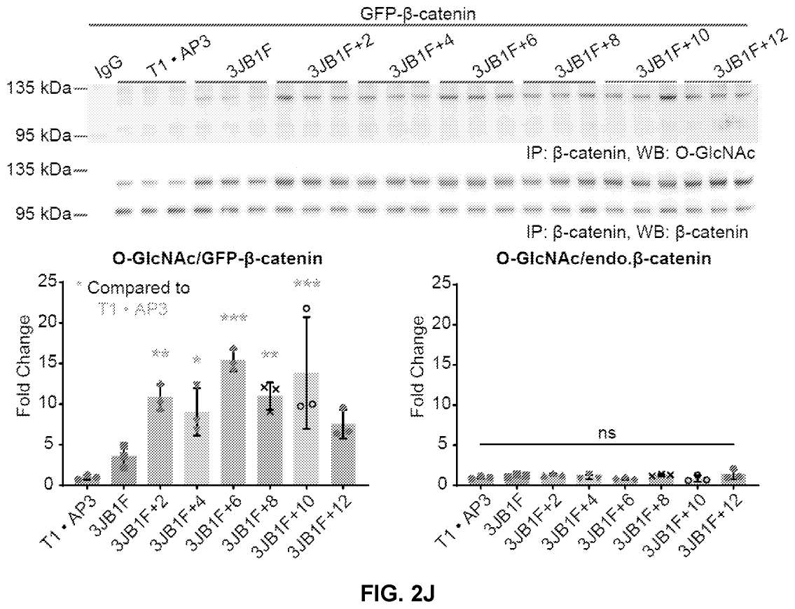

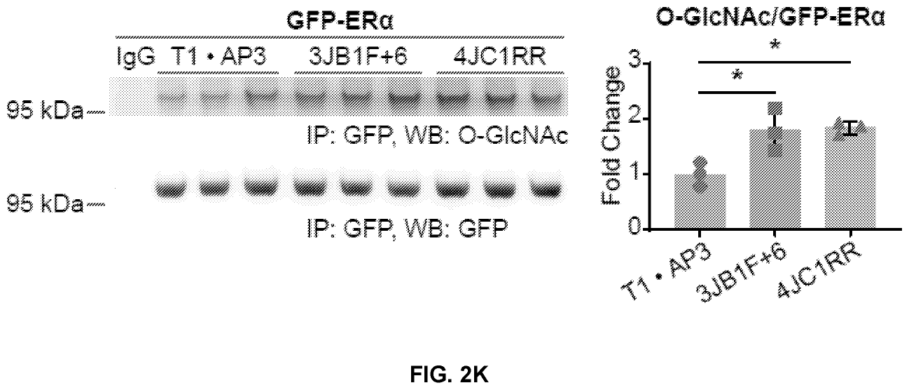

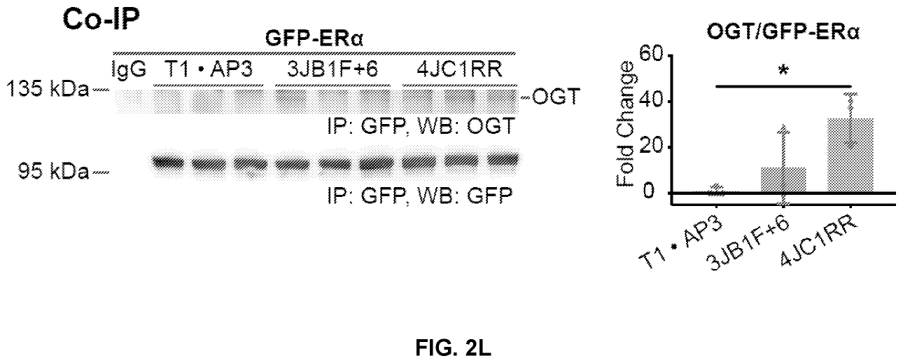

Next, T1 was conjoined with AP3, an RNA aptamer for GFP (Shui, B., et al. Nucleic Acids Res 2012 40:e39). In these DS aptamers, T1 and AP3 were connected by a linker region. A series of flexible linkers were first designed that were single-stranded, GC-neutral, and that did not interfere with the folding of T1 and AP3. The U6 promoter was chosen to drive the expression of aptamers, thus five adenine residues were inserted upstream of AP3 to capture the transcribed U6 terminator (5-6 uracil residues) (FIG. 2A; Table 1). Like the individual T1 aptamer, the DS aptamer has a short half-life (8.029 min) in cells (FIG. 2B).

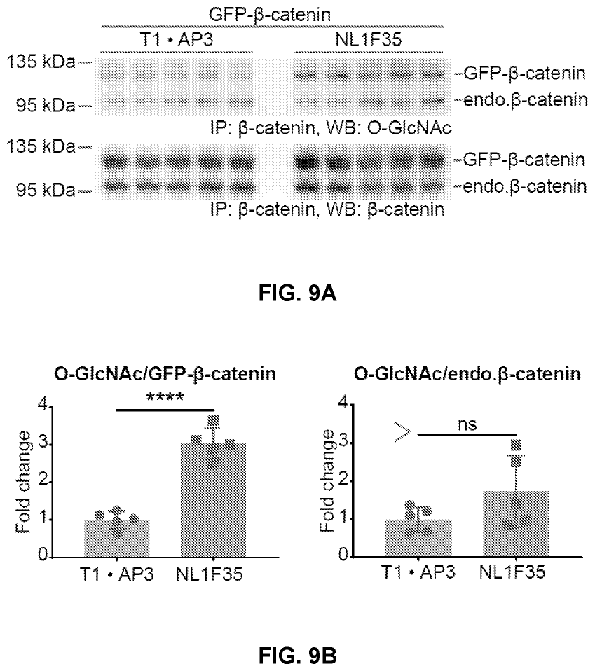

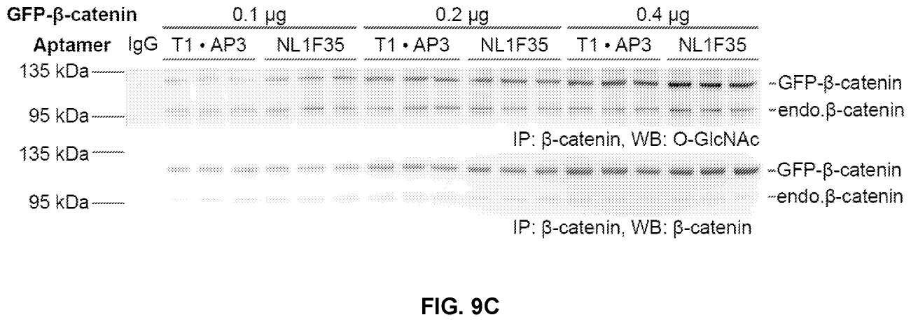

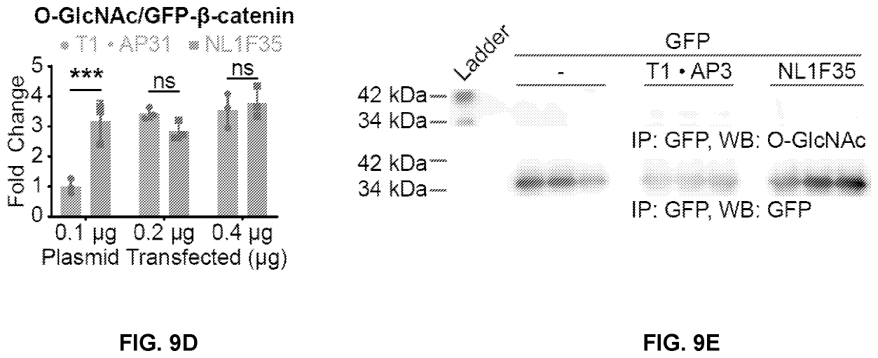

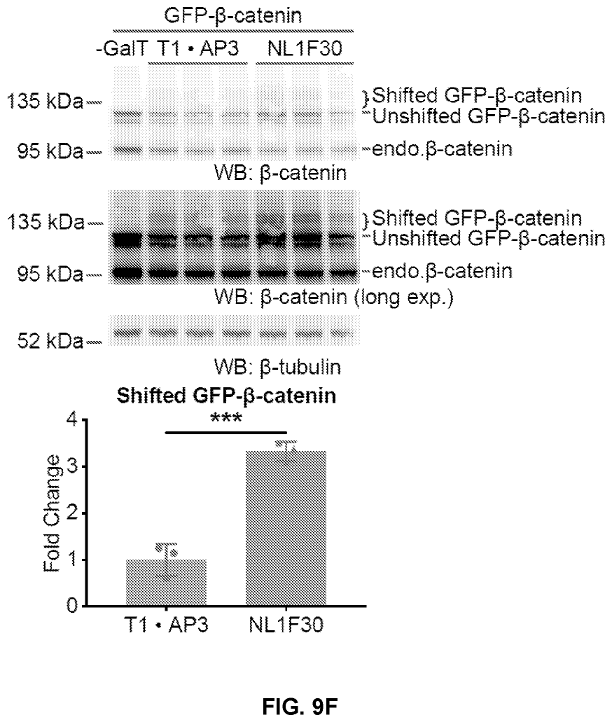

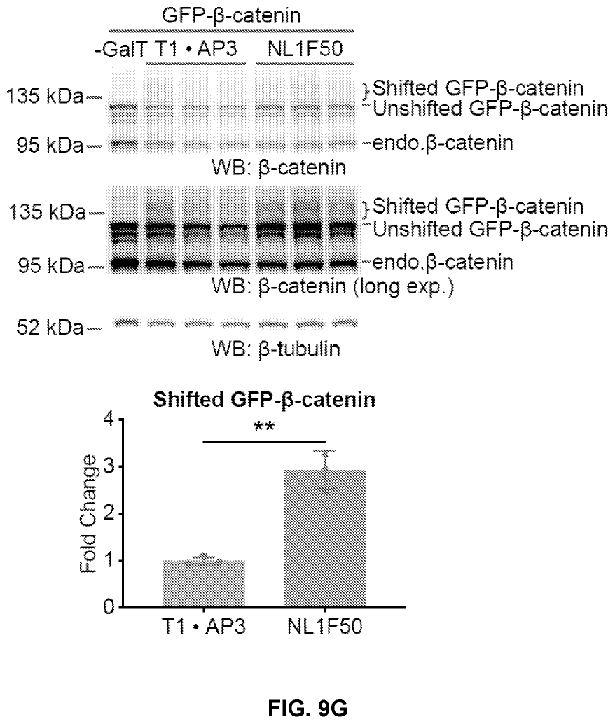

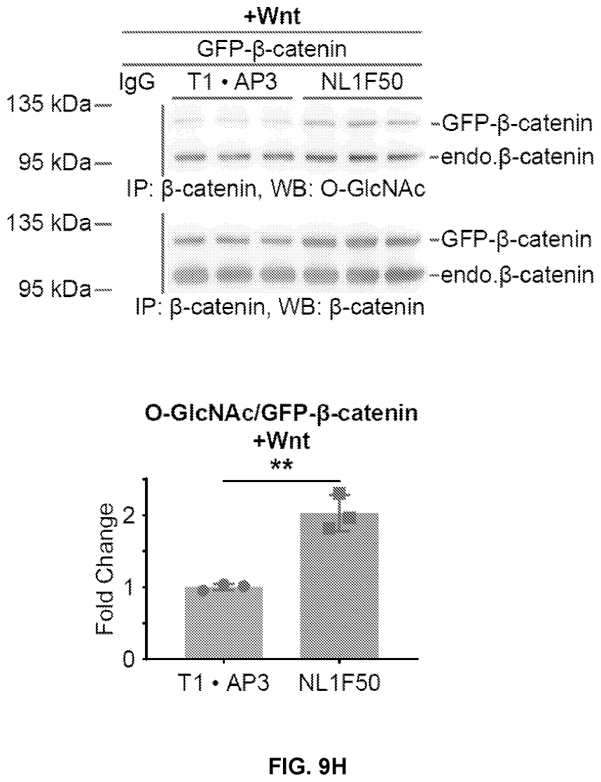

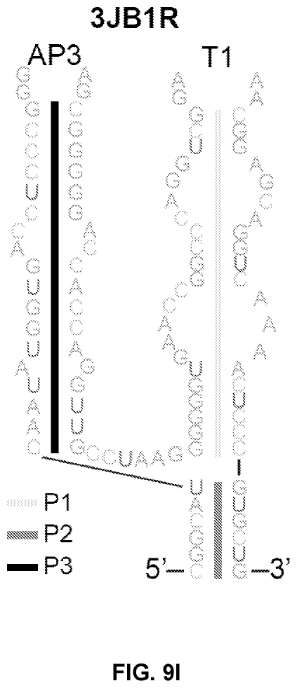

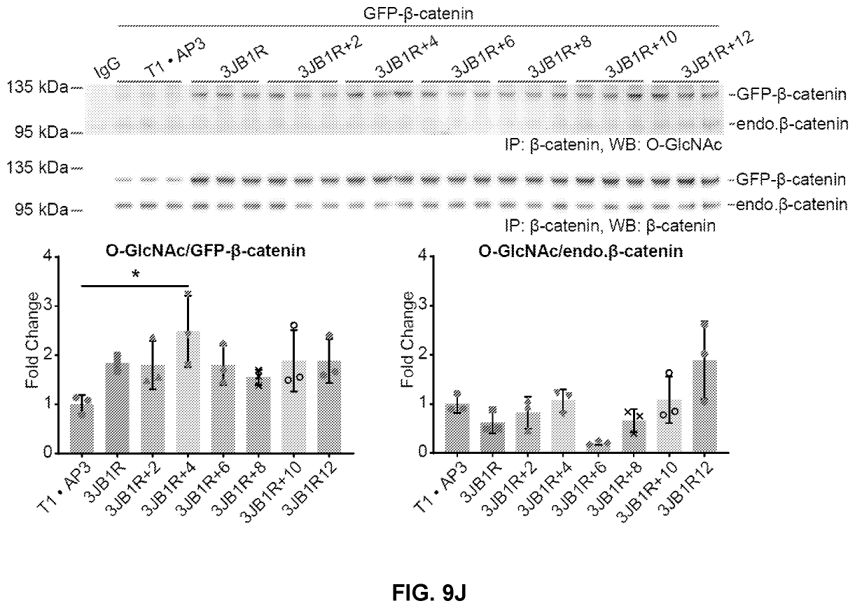

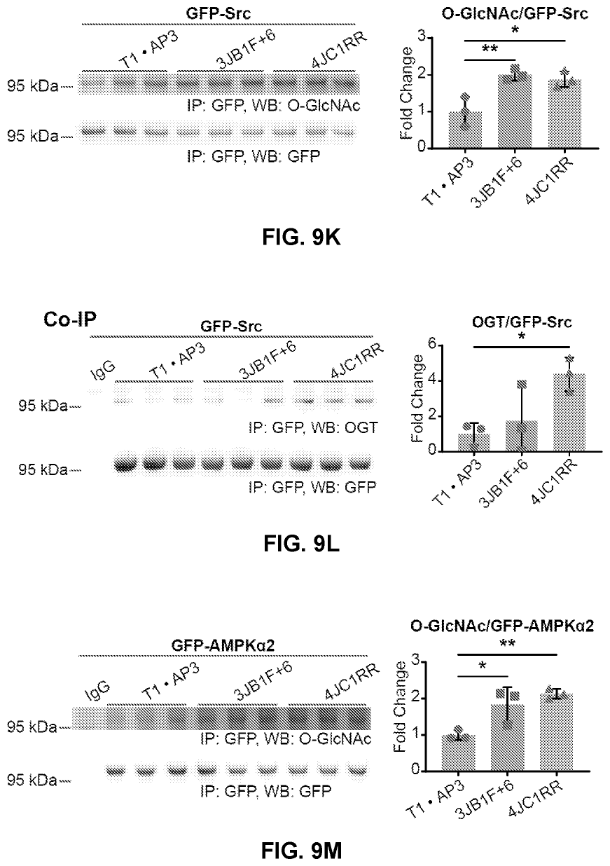

Expression of these NL1F DS aptamers significantly increased O-GlcNAcylation of GFP-β-catenin in a specific manner. Protein-specific O-GlcNAcylation was quantified by immunoprecipitation (IP) and Western Blot (WB). In comparison to the individual T1 and AP3 aptamers (T1·AP3), NL1F30, NL1F35 and NL1F50 up-regulated O-GlcNAc levels on GFP-β-catenin by 2- to 3-fold (FIGS. 2C, 2D, 9A to 9D). Efficacy of DS aptamers was further confirmed in mass-shift assays, where an O-GlcNAc group was enzymatically labeled with an N-azidoacetylgalactosamine (GalNAz), then conjugated to a polyethylene glycol (PEG) molecule of 8.5 kDa by Strain-promoted alkyne-azide cycloaddition (SPAAC), causing a mobility shift of the protein by 8.5 kDa per O-GlcNAc in WB (Darabedian, N., et al. Biochemistry 2018 57:5769-5774). In mass-shift assays, NL1F30 and NL1F50 increased O-GlcNAcylation on GFP-β-catenin by 3.3-fold and 2.9-fold, respectively (FIGS. 9F, 9G). Increasing the expression of GFP-β-catenin renders NL1F35 ineffective, suggesting that the molar ratio of aptamer to substrate is important for the efficacy of DS aptamers (FIGS. 9C, 9D). Meanwhile, O-GlcNAc on endogenous β-catenin remained unchanged (FIGS. 2C, 2D, 9A, 9B), and DS aptamers did not affect global O-GlcNAcylation (FIG. 2E). These results indicate good selectivity of the DS aptamers. The protein abundance of GFP-β-catenin was also increased, suggesting a stabilizing effect of O-GlcNAc on this protein (FIG. 2E). DS aptamer increased O-GlcNAcylation on GFP-β-catenin in Wnt-stimulated cells (FIG. 9H). On GFP-β-catenin, the changes in O-GlcNAcylation arose from its β-catenin portion, as O-GlcNAc on the GFP tag was undetectable (FIG. 9E).

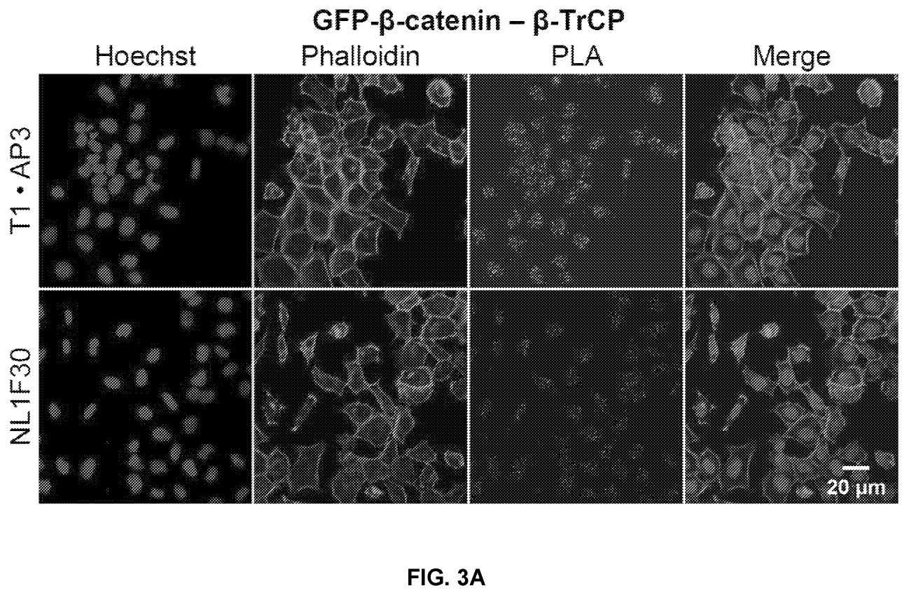

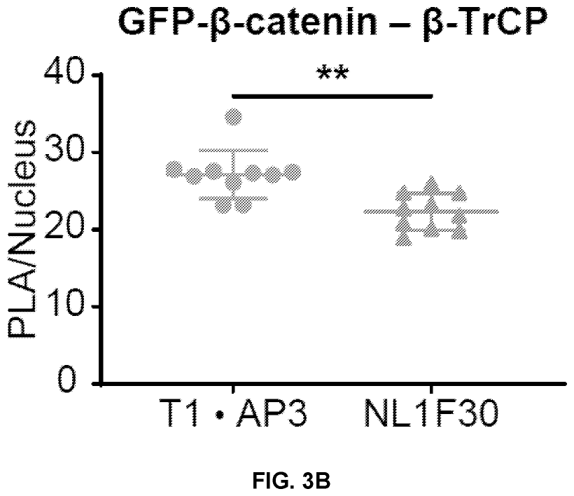

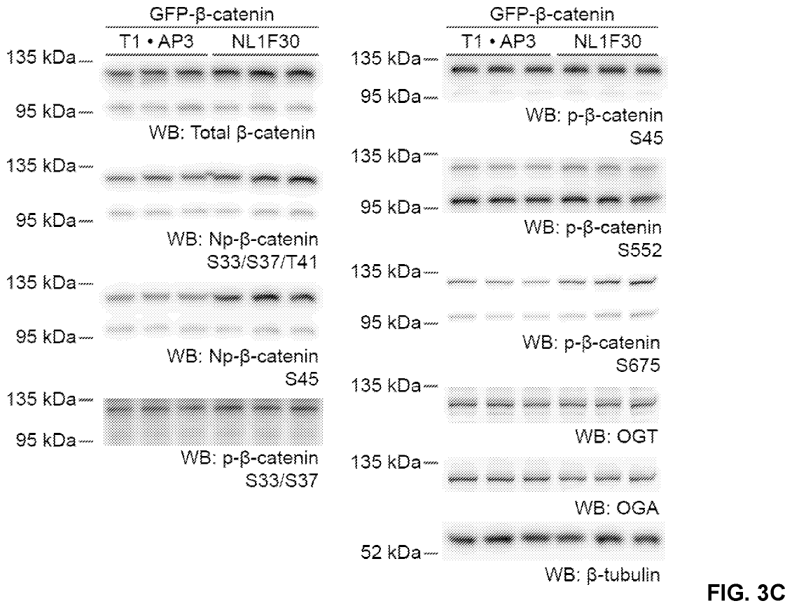

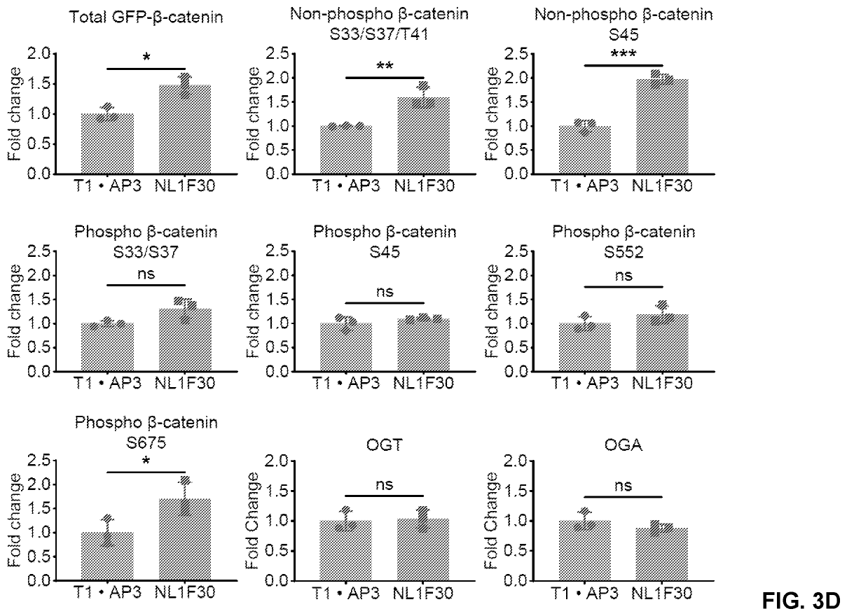

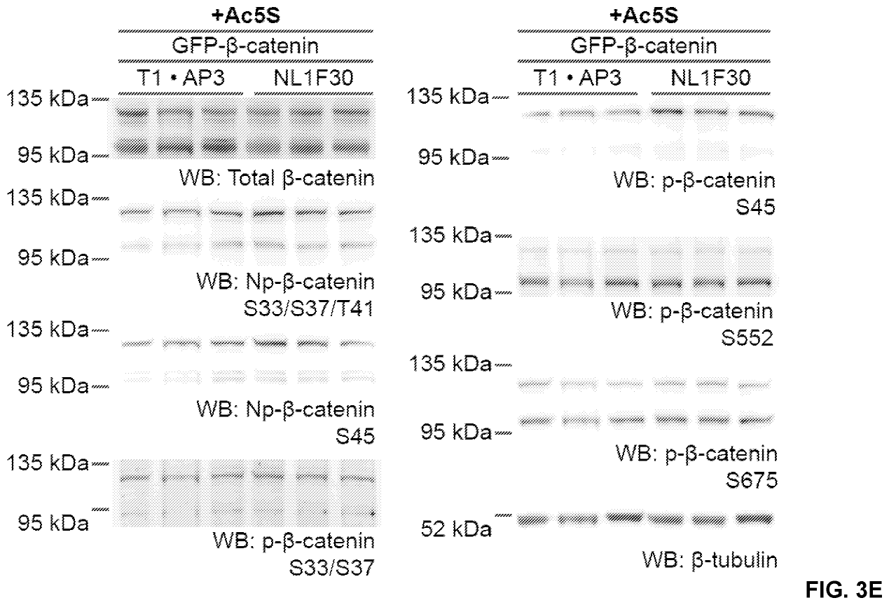

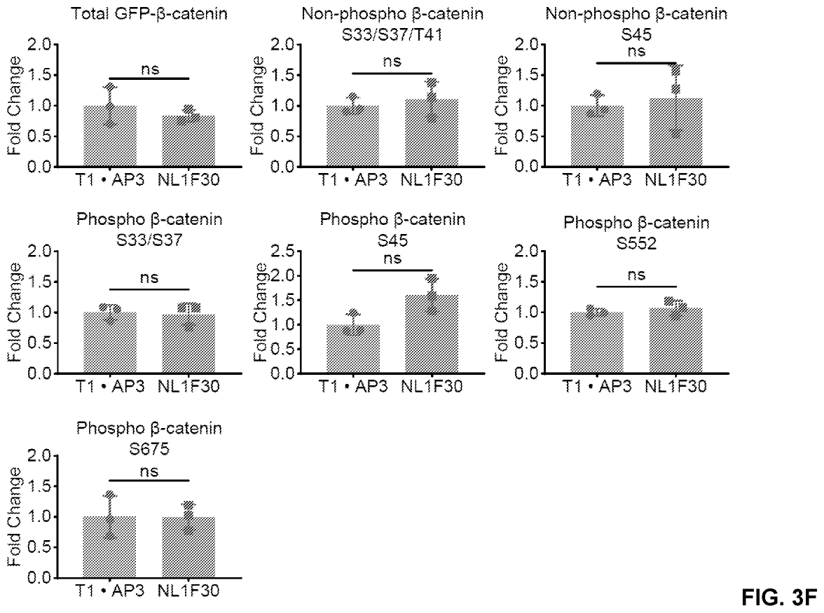

Ac₄-5SGlcNAc (Ac5S) and thiamet-G (TMG) are highly potent inhibitors of OGT and OGA, respectively (Yuzwa, S. A., et al. Nat Chem Biol. 2008 4(8):483-90; Gloster, T. M., et al. Nat Chem Biol 2011 7:174-181). DS aptamers did not induce hyper-O-GlcNAcylation on GFP-β-catenin in Ac5S-treated cells but increased this 0-GlcNAcylation in TMG-treated cells (FIGS. 2F and 2G). Thus, the activity of OGT is required for aptamer-induced hyper-O-GlcNAcylation, while the activity of OGA is not. These observations, together with the observation that the DS aptamers do not change the abundance of OGT and OGA (FIGS. 3C, 3D), rule out the possibility that aptamer-induced hyper-O-GlcNAcylation arise from the potential “off-target” effects of RNA aptamers.

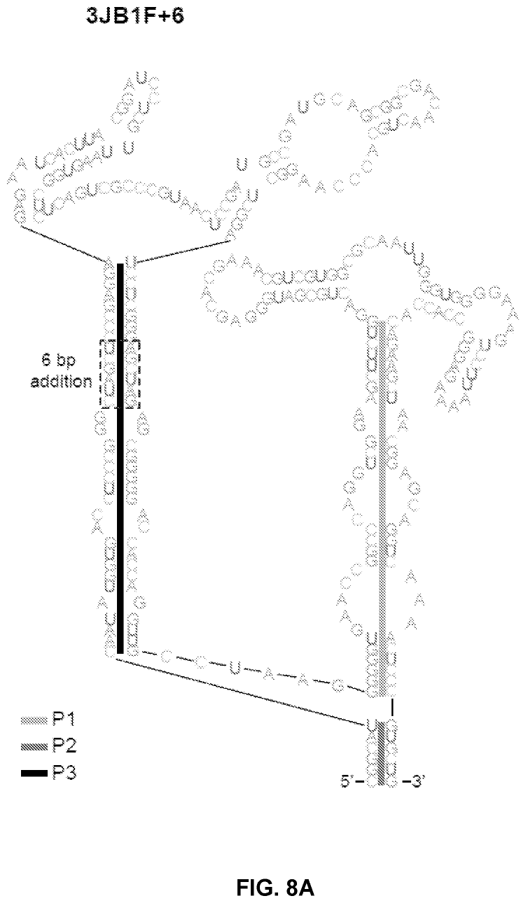

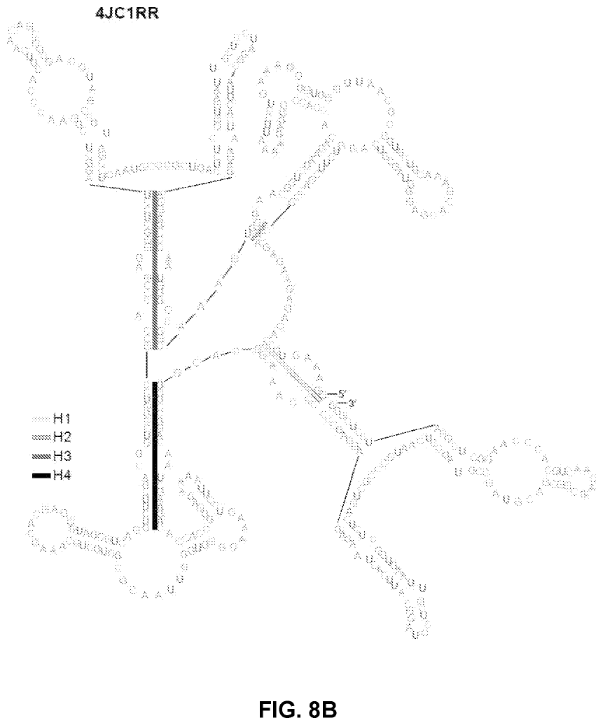

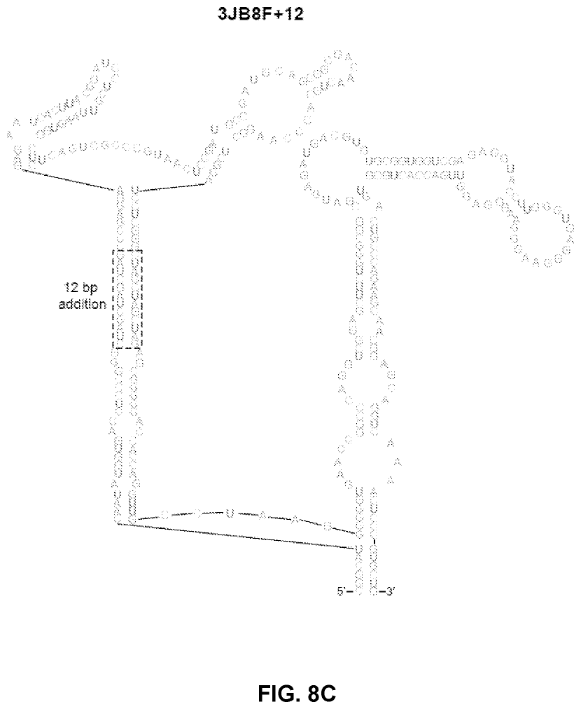

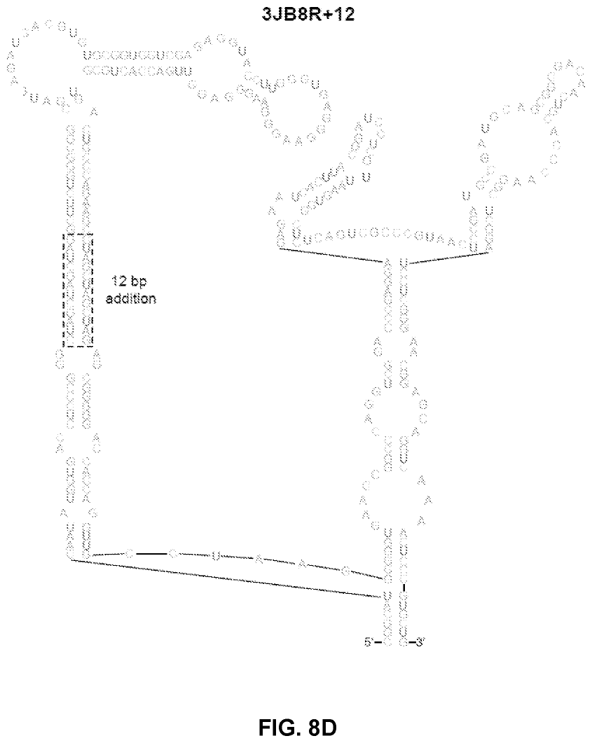

In addition to the single-stranded flexible linkers, DS aptamers were designed with folded linkers. Three-way and four-way RNA junctions are naturally occurring RNA structures. In family C of three-way junctions, the P1 and P2 helices are coaxially stacked while the P3 helix “bends” towards P1 (FIG. 2H) (Lescoute, A., et al. RNA 2006 12:83-93). These structural properties result in proximity between the two helices, allowing us to design DS aptamers with folded linkers. We picked a structure (PDB 1MFQ, S-domain of the human 7SL RNA) from this family (Kuglstatter, A., et al. Nat Struct Biol 2002 9:740-744) and grafted T1 and AP3 onto its helices. By swapping the locations of T1 and AP3, two DS aptamers (3JB1F and 3JB1R) were generated (FIGS. 2H, 9I; Table 1). On 3JB1F and 3JB1R, T1 and AP3 were of identical distance but different orientations. To further optimize the folded linkers, serial additions were introduced into the P3 helices of 3JB1F and 3JB1R (FIG. 8A; Table 1). Considering that in RNA double helices (A-form), addition of 1 bp introduces a rotation of 32.7° but only increases the length of the helix by 2.8 Å (Tanaka, Y., et al. Nucleic Acids Res 1999 27:949-955), these additions cause notable changes in the orientation of T1/AP3, but minor variations on the distance.

The 3JB1F variants significantly promoted O-GlcNAcylation on GFP-β-catenin (FIG. 2J). The original 3JB1F increased this PTM by 3.5-fold, which was dramatically improved by serial additions on its P3 helix. Most of the variants increased O-GlcNAc on GFP-β-catenin by more than 10-fold. Among them, the variant with 6 bp addition (3JB1F+6; FIG. 8A) increased this PTM by 15-fold. Notably, the impact of serial additions showed some “periodicity”, which peaked at 6 bp and decreased as additions grew longer. This observation agrees with the structural properties of RNA helices, where addition of 11 bp introduces a 360° rotation. Meanwhile, O-GlcNAcylation of endogenous β-catenin remained unchanged (FIG. 2J). 3JB1R increased O-GlcNAcylation of GFP-β-catenin by 2-fold, however, serial additions had little effect (FIG. 9J). This observation indicates that on 3JB1R, the orientation of T1 is suboptimal, so that OGT is facing away from its target protein when bound to the complex.

DS aptamers increase O-GlcNAc levels on other GFP-tagged proteins. Here another folded linker was designed. In family π of four-way RNA junctions, the helices H3 and H4 are coaxially stacked while H1 and H2 “swings”, and H2 is attracted by H3 (FIG. 2I) (Laing, C., et al. J Mol Biol 2009 390:547-559). A structure (PDB 1 U9S, the specificity domain of A-type ribonuclease P) was picked from this family (Krasilnikov, A. S., et al. Science 2004 306:104-107), and grafted two copies of T1 and AP3 onto its helices (FIG. 8B; Table 1). This 4JC1 RR, as well as 3JB1F+6, increased O-GlcNAc on GFP-tagged Estrogen Receptor α (ERα), Src kinase and AMP-activated protein kinase α2 (AMPKa2) (FIGS. 2K, 9K, 9M). Co-immunoprecipitation (Co-IP) revealed that 4JC1RR promoted the interaction between OGT and these proteins (FIGS. 2L, 9L), which supports our hypothesis that DS aptamers increase O-GlcNAcylation by inducing proximity between OGT and its substrates.

In conclusion, DS aptamers with flexible or folded linkers promote interactions between OGT and GFP-tagged proteins and specifically increase O-GlcNAcylation only on these substrates.

### DS Aptamers Reveals Novel O-GlcNAc Sites on β-Catenin

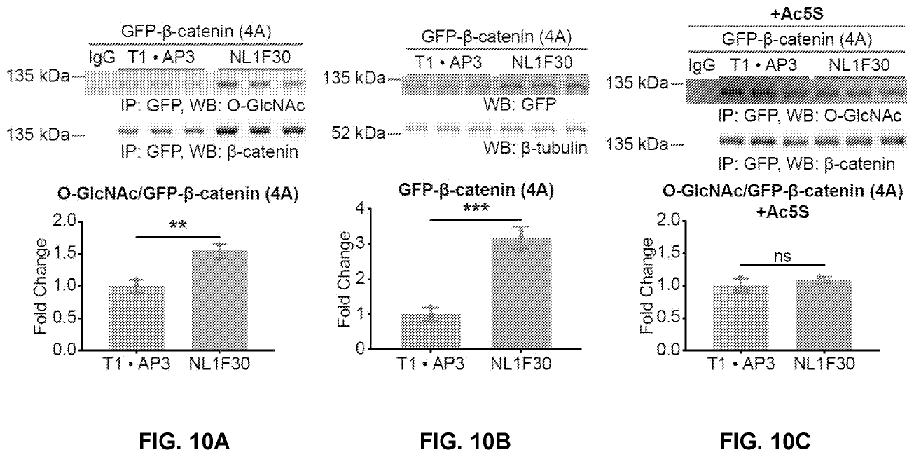

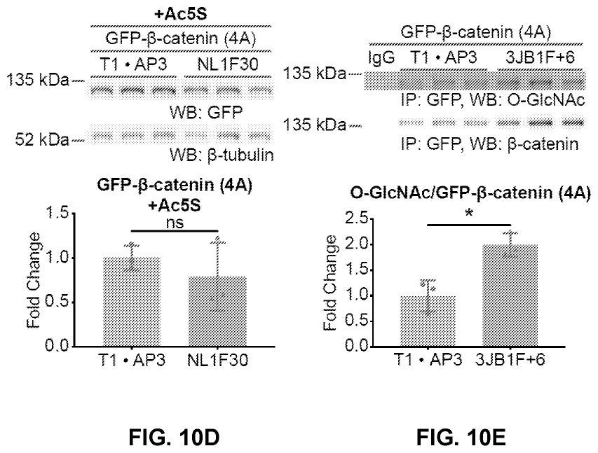

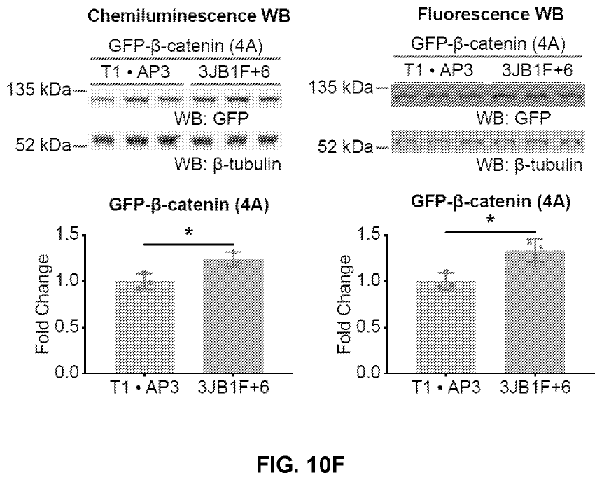

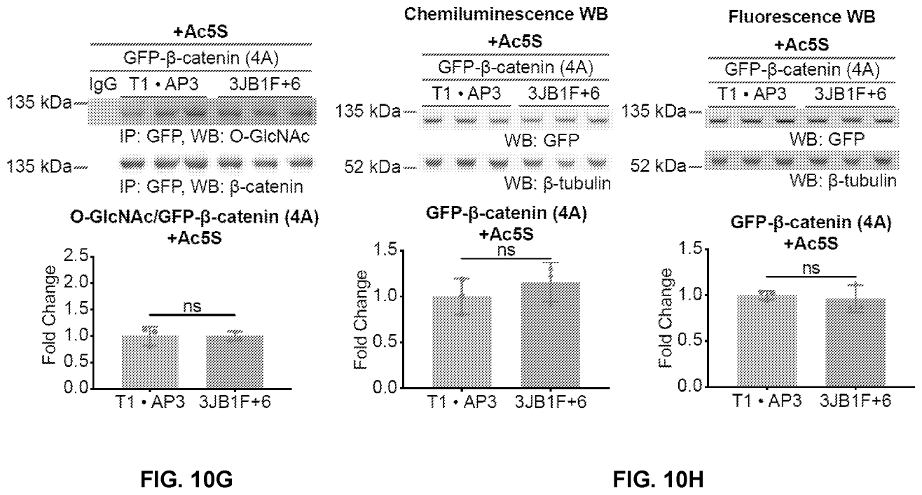

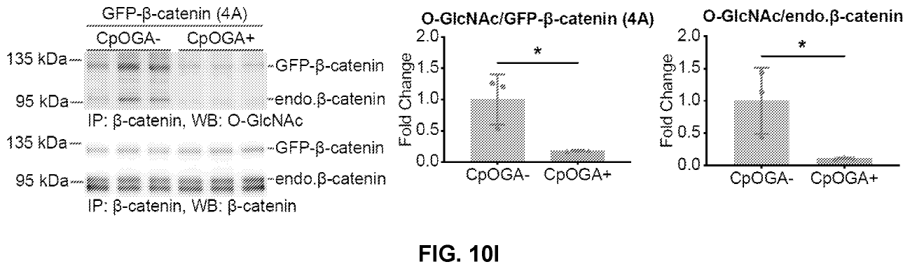

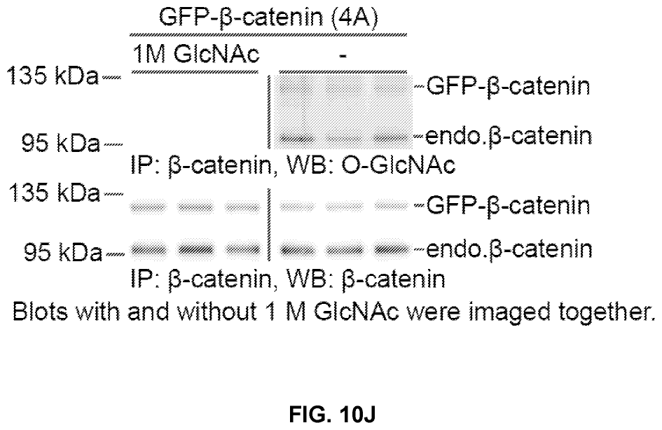

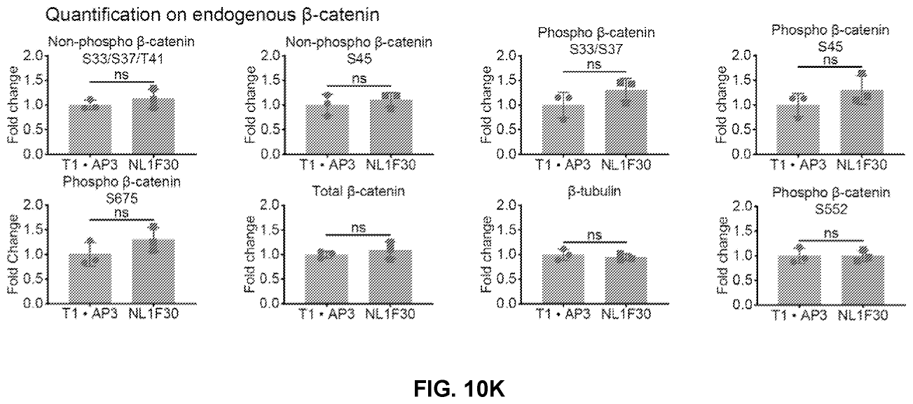

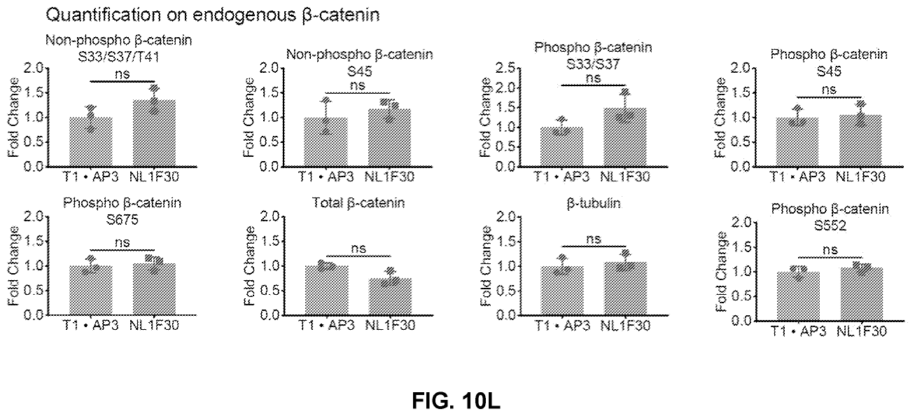

This study indicates unidentified O-GlcNAc sites on β-catenin. Four O-GlcNAc sites have been mapped (Ser23, Thr40, Thr41 and Thr112) (Olivier-Van Stichelen et al., 2014). When all these sites were mutated to alanine, O-GlcNAcylation on the GFP-β-catenin (4A) mutant was still induced by NL1F30 or 3JB1F+6, though to a lesser extent than on the wild-type protein (FIGS. 10A, 10E, 2C, 2J). This induction required the activity of OGT (FIGS. 10C, 10G). Similar to on the wild-type protein, O-GlcNAc stabilized the 4A mutant. Interestingly, though NL1F30 did not induce higher O-GlcNAcylation than 3JB1F+6, it was more effective on stabilizing the protein (FIGS. 10B, 10F). The stabilization effects required the activity of OGT (FIGS. 10D, 10H). These observations suggest that NL1F30 and 3JB1F+6 have different site-selectivity when inducing O-GlcNAcylation on GFP-β-catenin, and that O-GlcNAc on the unidentified sites regulates the stability of β-catenin.

In control experiments, O-GlcNAc signal on the 4A mutant was reduced by a bacterial homolog of OGA (CpOGA) (Rao, F. V., et al. EMBO J 25:1569-1578), or by competing the O-GlcNAc antibody with 1 M GlcNAc during WB (FIGS. 10I, 10J), confirming the signal was specific to O-GlcNAc.

O-GlcNAc Stabilizes GFP-β-Catenin by Inhibiting its Interaction with β-TrCP

As the key transcription factor of the Wnt signaling pathway, β-catenin is activated by Wnt. Without Wnt, newly synthesized β-catenin is recruited to a destruction complex comprised of Axin, APC, GSK3P and CK1. In the complex, β-catenin is consecutively phosphorylated by CK1 and GSK3P on its Ser45, Thr41, Ser37 and Ser33 residues, ubiquitinated by the SCF^{β-TrcP} complex and degraded by proteasomes (Hart, M., et al. Current Biology 1999 9:207-211; ter Haar, E., et al. Nat Struct Biol 2001 8:593-596; Liu, C., et al. Cell 2002 108:837-847). Binding of Wnt to the membrane receptor Frizzled triggers a cascade of signaling events, which ultimately blocks the ubiquitination of phosphorylated β-catenin in the destruction complex (Li, V. S., et al. Cell 2012 149:1245-1256). Stabilized β-catenin saturates the complex, accumulates in the cytoplasm, enters the nucleus, and activates transcription (Clevers, H., et al. Cell 2012 149:1192-1205).

β-TrCP recruits β-catenin that is phosphorylated on Ser33 and Ser37 to the SCF^{β-TrCP} E3 ubiquitin ligase, causing its ubiquitination and degradation (Hart, M., et al. Current Biology 1999 9:207-211; Liu, C., et al. Proc Natl Acad Sci USA 1999 96:6273-6278). In Proximity Ligation Assays (PLA), DS aptamer considerably inhibited the interaction between GFP-β-catenin and β-TrCP (FIGS. 3A, 3B). It was reasoned that impairing this interaction would inhibit the degradation of phosphorylated GFP-β-catenin, therefore saturate the destruction complex and stabilize the newly synthesized, unphosphorylated GFP-β-catenin. DS aptamer increased the non-phosphorylated moiety of this protein, regarding Ser33, Ser37, Thr41 and Ser45 (FIGS. 3C, 3D), agreeing with our hypothesis. Meanwhile, abundance of phosphorylated GFP-β-catenin was unchanged regarding these sites (FIGS. 3C, 3D), suggesting that O-GlcNAc inhibit the interaction between GFP-β-catenin and β-TrCP not by competing phosphorylation, but likely by steric hinderance since it has a large Stokes radius (Hart, G. W. J Biol Chem 2019 294:2211-2231). Additionally, DS aptamer increased phosphorylation on Ser675 (FIGS. 3C, 3D) that enhances the transcriptional activity of β-catenin (Taurin, S., et al. J Biol Chem 2006 281:9971-9976). These site-specific phosphorylations on endogenous β-catenin were not changed (FIGS. 3C, 10K). Changes of site-specific phosphorylation required the activity of OGT (FIGS. 3E, 3F, 10L), supporting that they were caused by aptamer-induced O-GlcNAcylation.

In conclusion, O-GlcNAc on GFP-β-catenin inhibits its recognition by β-TrCP, thus stabilizes this protein (FIGS. 2E, 3C, 3D, 10B, 10F).

### Dual-Specificity Aptamers Increase O-GlcNAcylation on Endogenous β-Catenin

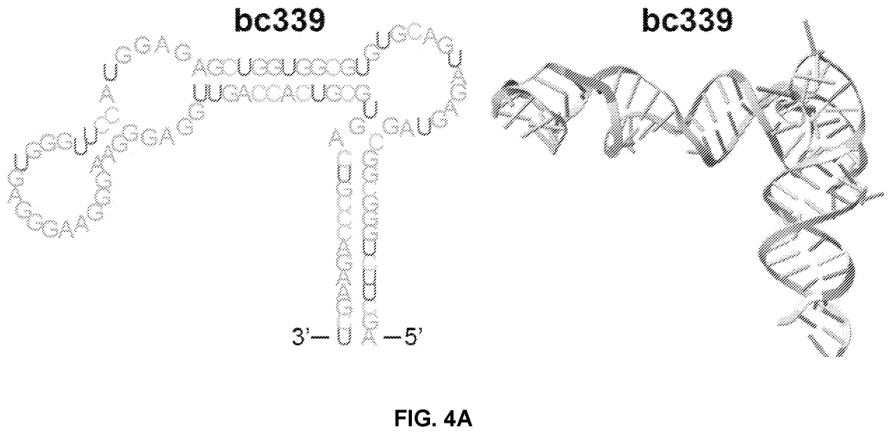

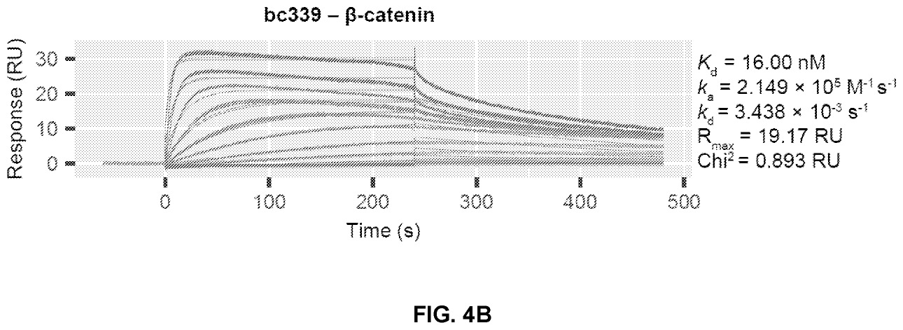

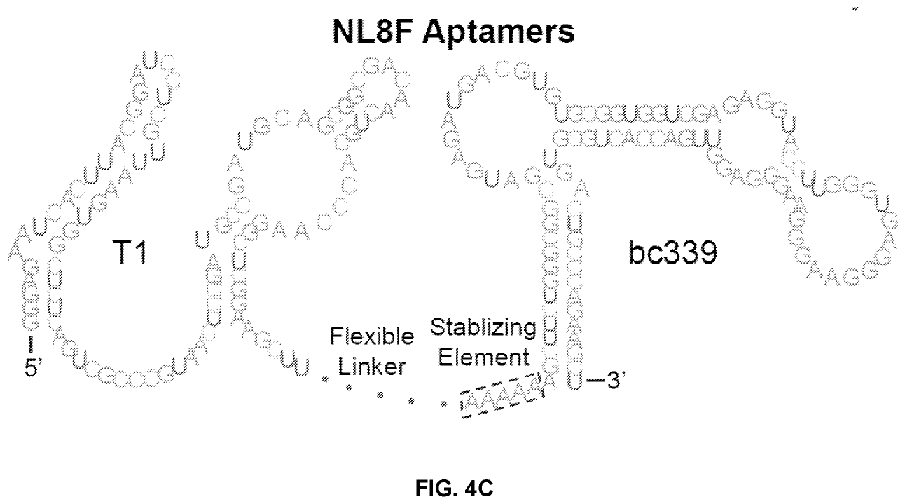

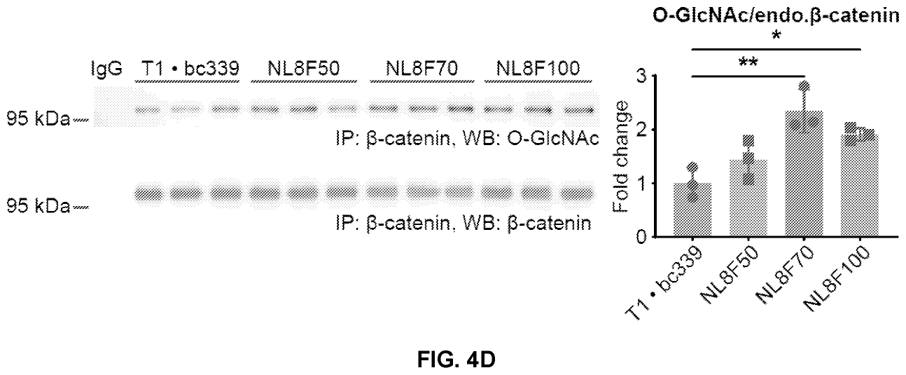

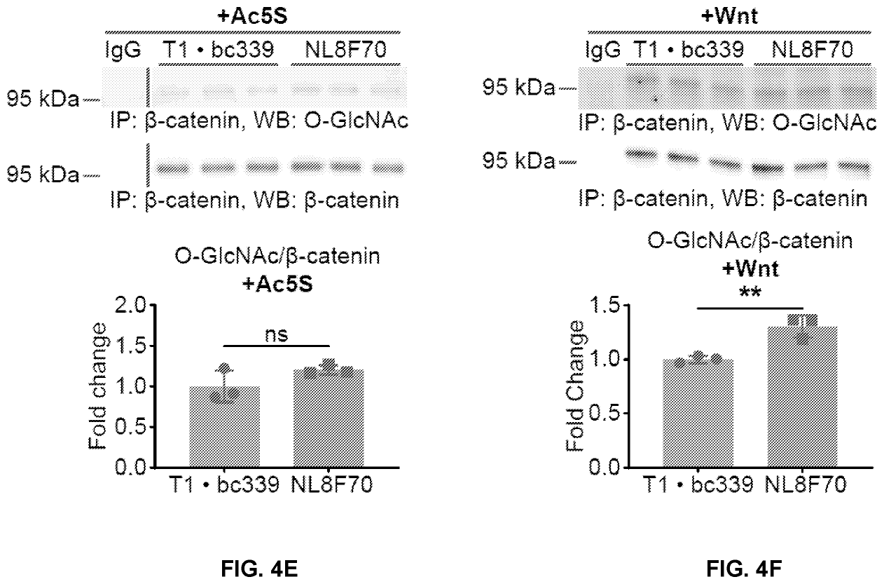

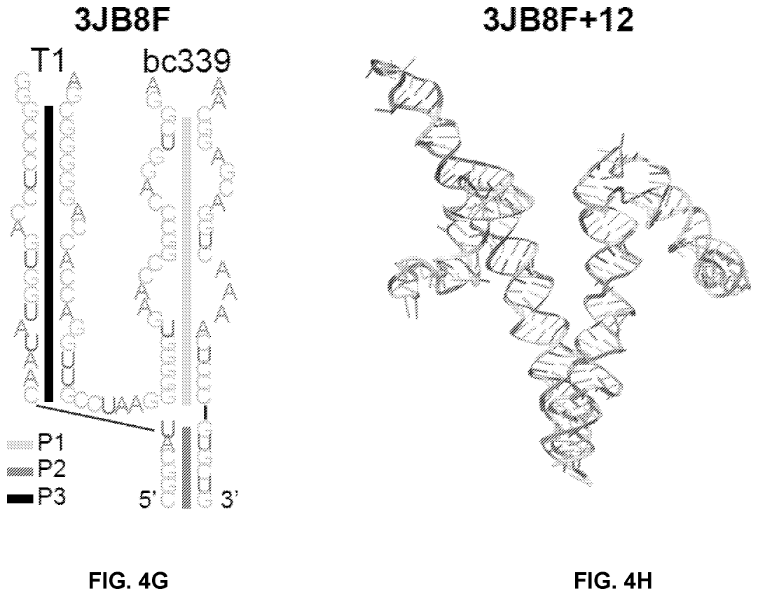

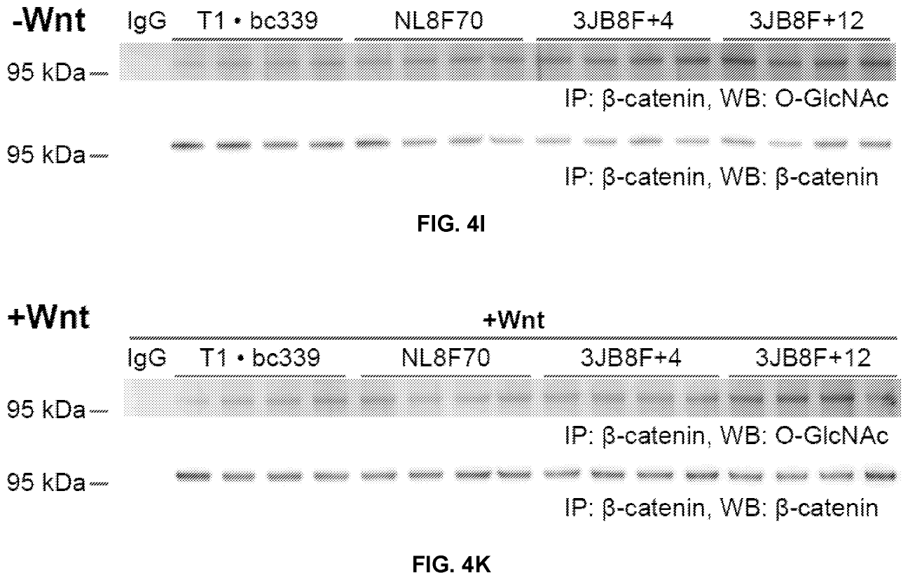

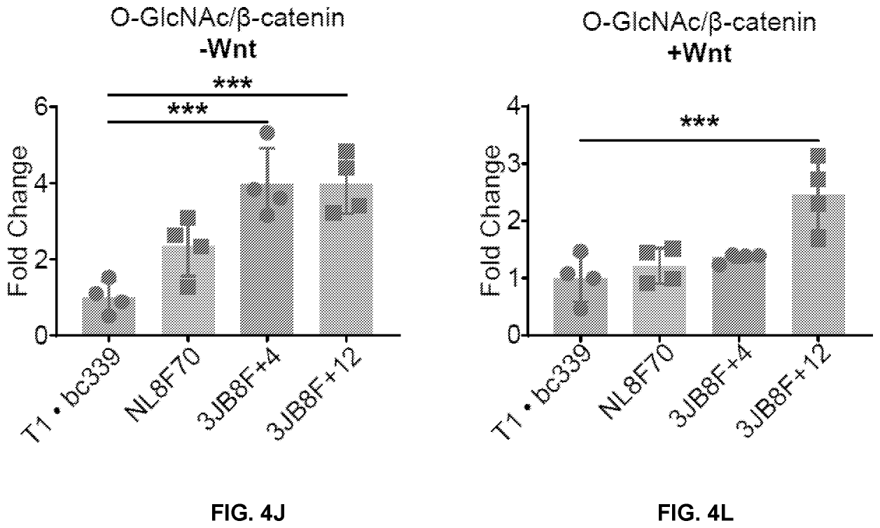

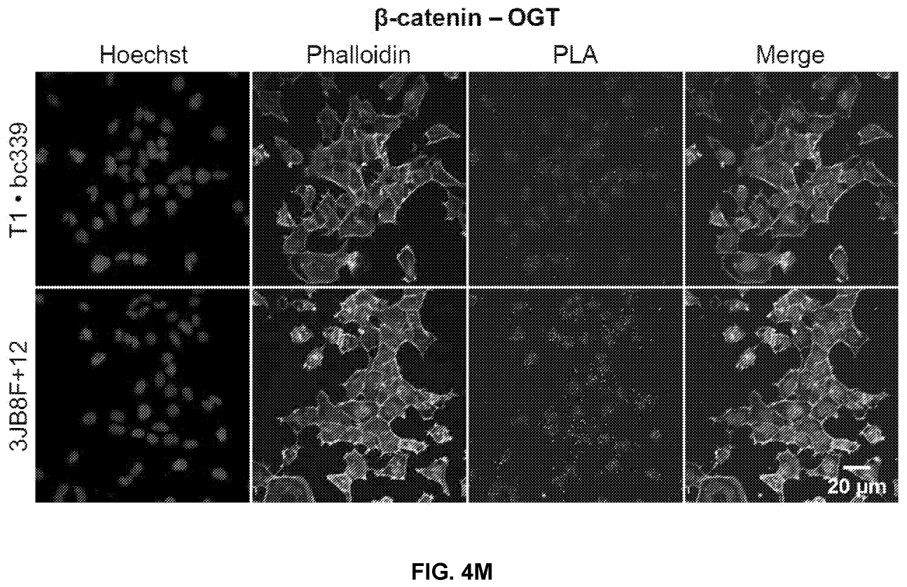

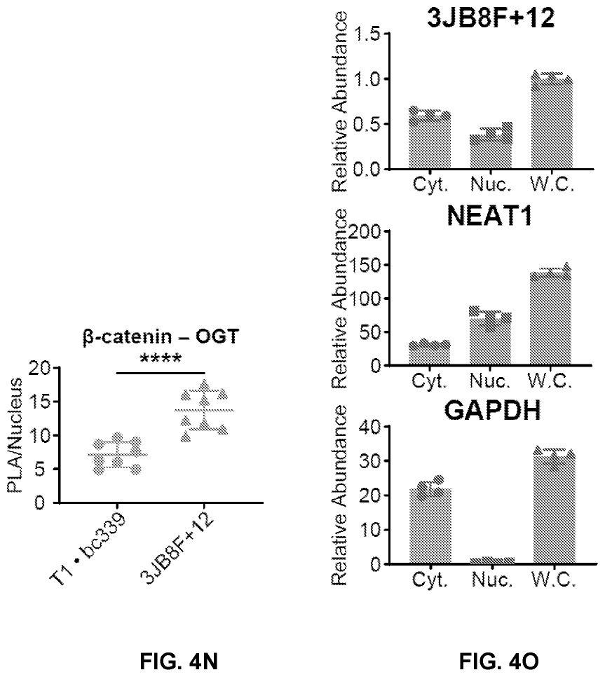

Beyond epitope-tagged proteins, the question was whether DS aptamers could regulate O-GlcNAc on endogenous proteins. An RNA aptamer (bc339) targeting β-catenin was generated from SELEX (FIG. 4A; Tables 1 and 2). Its binding properties were characterized by SPR (FIG. 4B). The dissociation constant (K_{d}) between bc339 and β-catenin is 16.00 nM. This aptamer (bc339) was connected to T1 with a series of flexible linkers, thus the NL8F DS aptamers were designed (FIG. 4C; Table 1).

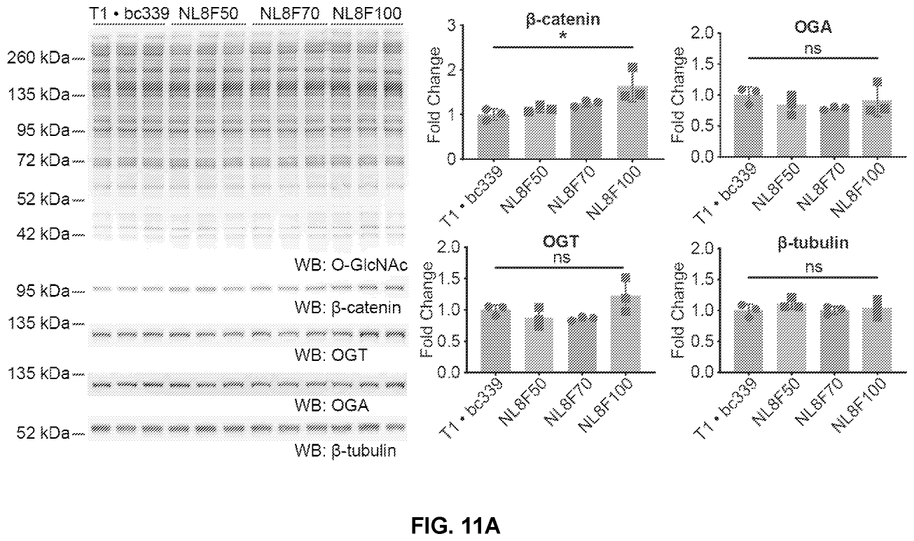

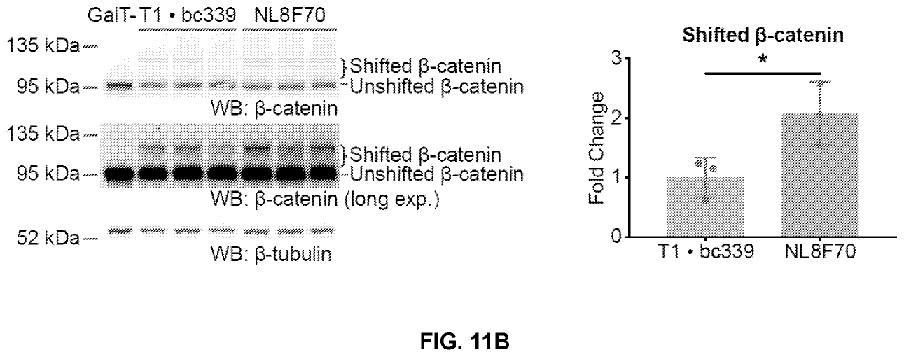

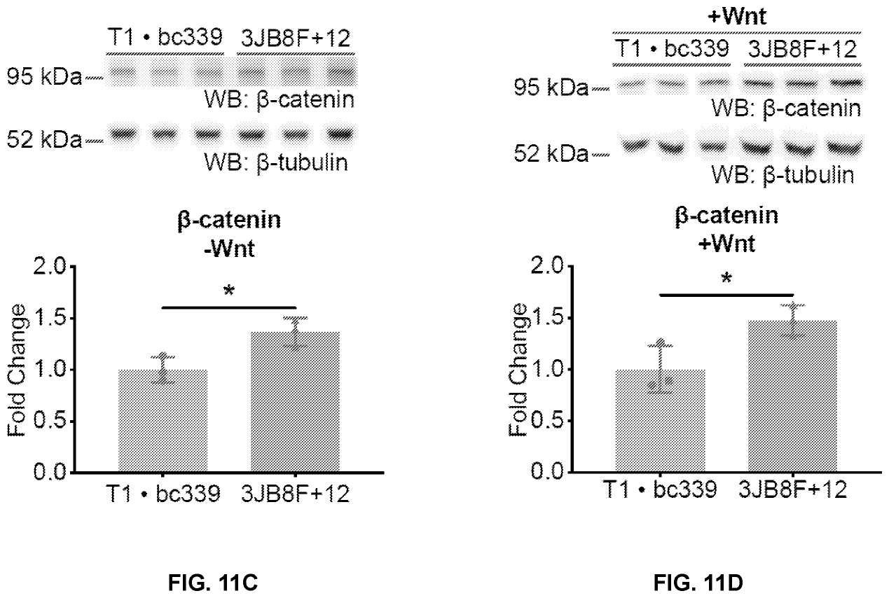

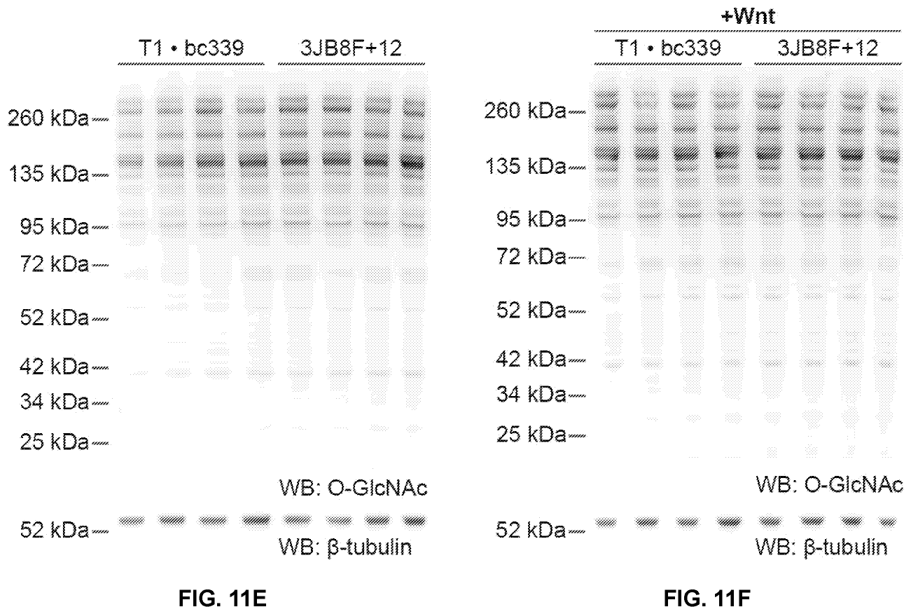

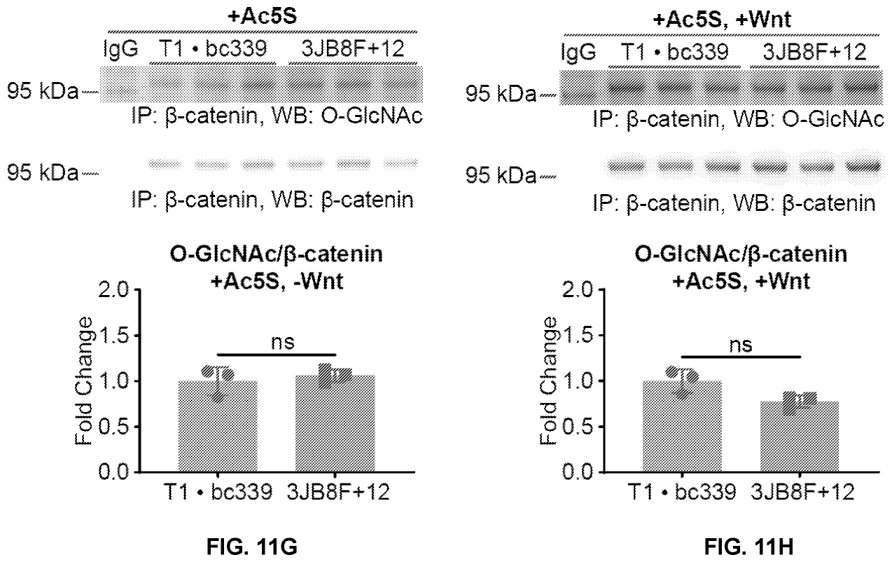

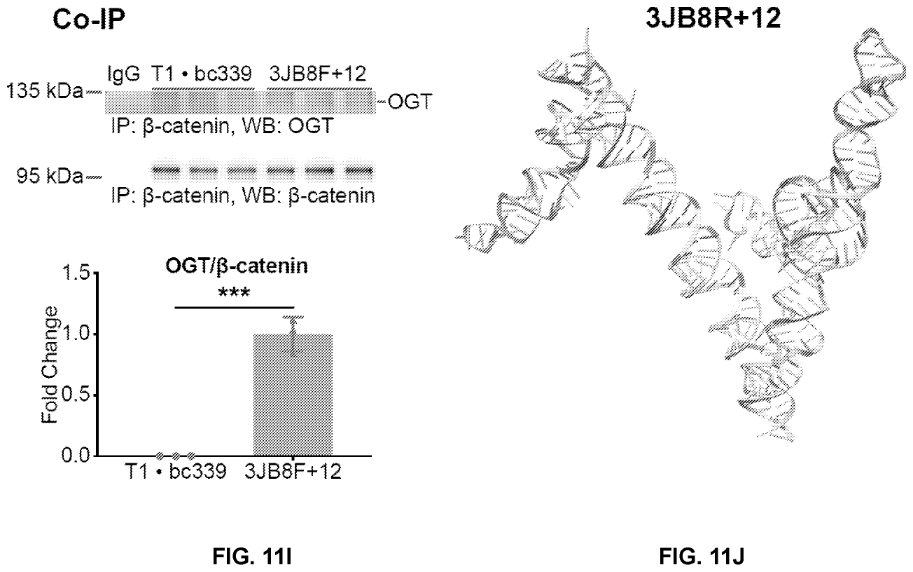

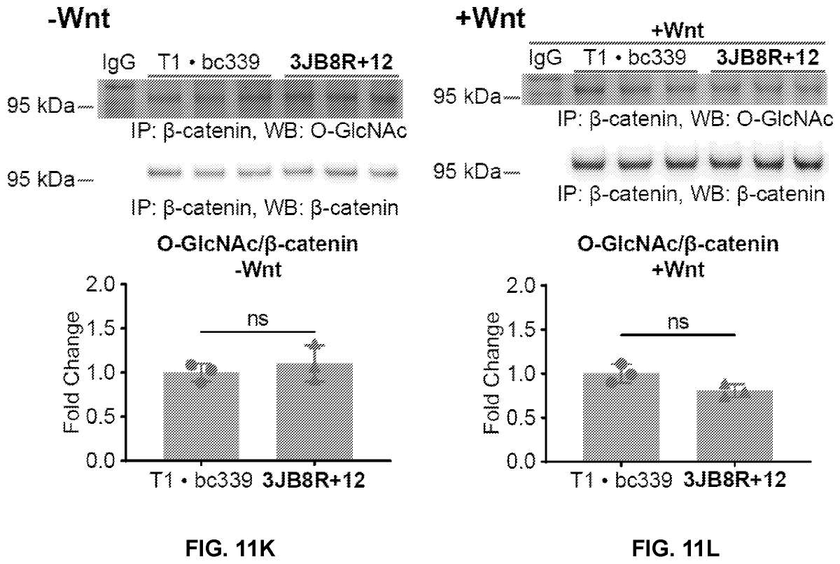

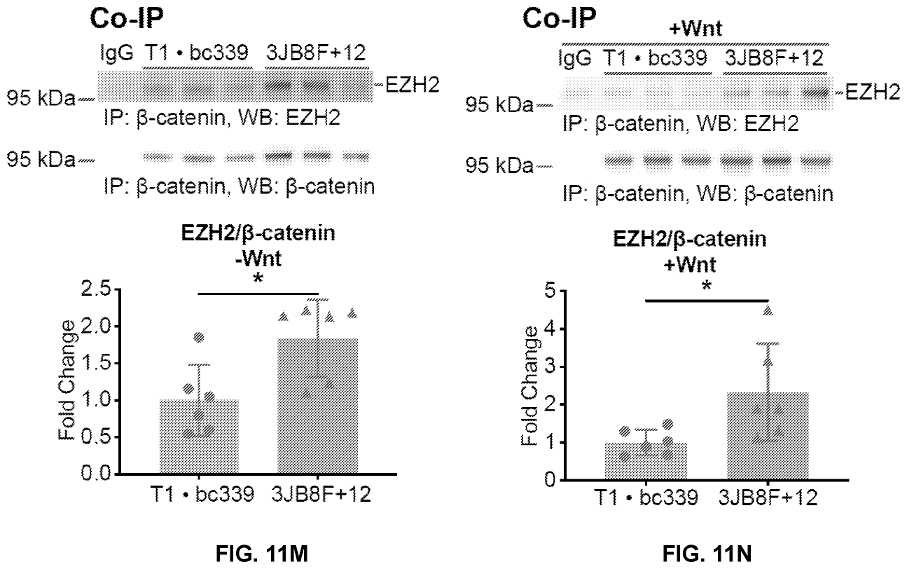

Expression of NL8F70 and NL8F100 increased O-GlcNAcylation of endogenous β-catenin by 2.3- and 1.9-fold, respectively (FIG. 4D). The abundance of β-catenin was increased by NL8F100 (FIG. 11A). Meanwhile, global O-GlcNAcylation, as well as the abundance of OGT and OGA, remained unchanged (FIG. 11A). These results reveal good selectivity of the DS aptamers. Treating cells with Ac5S prevented aptamer-induced O-GlcNAcylation (FIG. 4E). These data show that DS aptamers require the activity of OGT to increase O-GlcNAcylation of β-catenin. In mass-shift assay, NL8F70 increased O-GlcNAcylation on β-catenin by 2.1-fold (FIG. 11B). When Wnt signaling was activated, efficacy of NL8F70 became controversial (FIGS. 4F, 4K, 4L).

DS aptamers were also constructed with folded linkers. By replacing the GFP aptamer (AP3) in the 3JB1F constructs with bc339, the 3JB8F constructs were obtained (FIGS. 4G, 4H, 8C; Table 1). Among them, 3JB8F+12 increased O-GlcNAcylation of β-catenin by 4-fold in the absence of Wnt (FIGS. 4I, 4J), and by 2.5-fold in the presence of Wnt (FIGS. 4K, 4L). It also increased the abundance of β-catenin (FIGS. 11C, S4D). Global O-GlcNAcylation was not changed by 3JB8F+12 under either condition (FIGS. 11E, 11F). Like other DS aptamers, 3JB8F+12 requires the activity of OGT to induce O-GlcNAcylation under both Wnt− and Wnt+ conditions (FIGS. 11G, 11H).

In PLA and Co-IP, 3JB8F+12 substantially promoted the interaction between OGT and β-catenin (FIGS. 4M, 4N, 11I). This observation supports our hypothesis that DS aptamers regulate O-GlcNAcylation by targeting a substrate protein to OGT. Moreover, we studied the subcellular localization of DS aptamers by nucleus/cytoplasm fractionation and RT-qPCR (FIG. 4O). Compared to the RNA species enriched in nucleus (NEAT1) and cytoplasm (GAPDH), 3JB8F+12 localizes in both. Therefore, DS aptamers could regulate O-GlcNAcylation on both nuclear and cytoplasmic proteins.

Another DS aptamer (3JB8R+12) was constructed by swapping the locations of T1 and bc339 on 3JB8F+12 (FIGS. 8D, 11J; Table 1). Even though it consists of identical modules, 3JB8R+12 was unable to regulate O-GlcNAcylation of β-catenin under either status of Wnt signaling (FIGS. 11K and 11L). This observation further supports the hypothesis that not only the distance, but also the orientations of the individual aptamers on the folded linker are critical to the efficacy of DS aptamers. Additionally, the 3JB8R constructs are ideal controls to the 3JB8F DS aptamers.

In conclusion, the T1-linker-bc339 DS aptamers specifically increase 0-GlcNAcylation on endogenous β-catenin regardless of the status of Wnt signaling by inducing proximity between OGT and β-catenin. They could induce O-GlcNAcylation in both nucleus and cytoplasm.

O-GlcNAc Regulates β-Catenin's Interactions with Epigenetic Modifiers

Next studied was how O-GlcNAc regulates the functions of β-catenin using DS aptamers. As a transcription factor that does not have a DNA binding domain, the functions of β-catenin heavily rely on its interactions with other proteins, including EZH2, KAT2A and EP300 (Hecht, A., et al. EMBO J 2000 19:1839-1850; Shi, B., et al. Mol Cell Biol 2007 27:5105-5119; Chen, J., et al. Mol Cell Biol 2010 30:5621-5635).

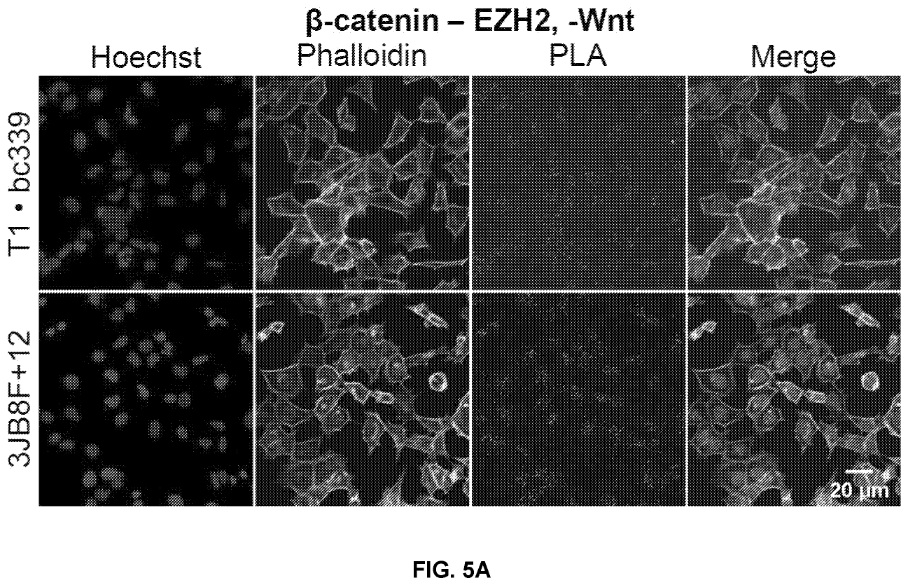

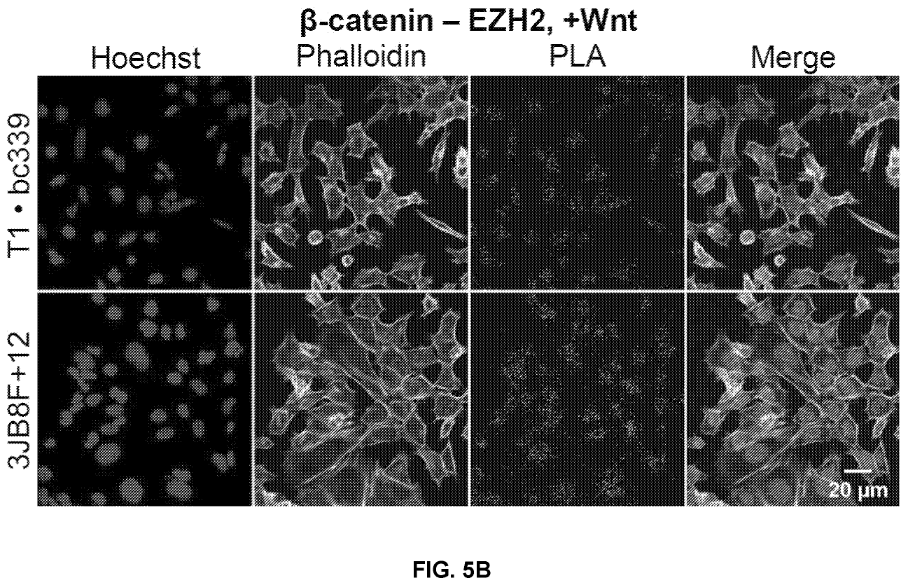

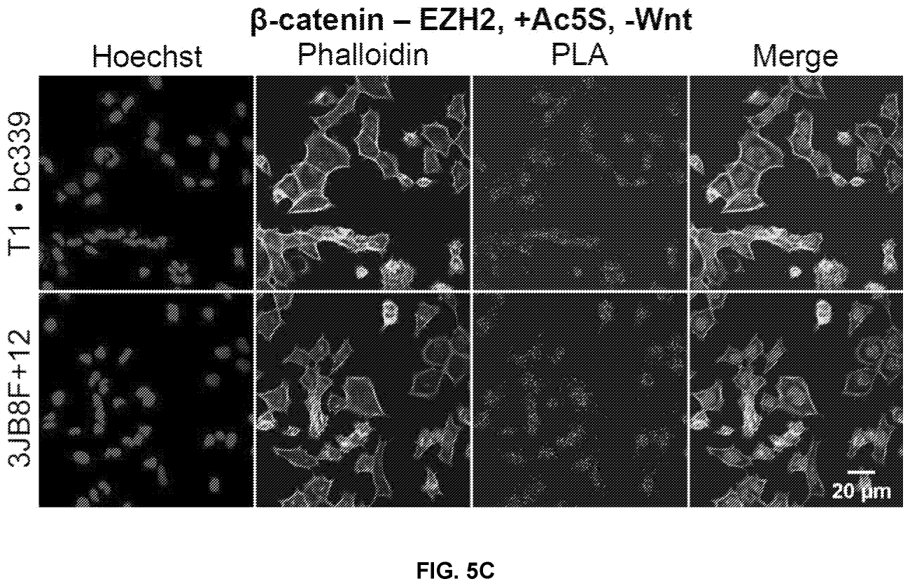

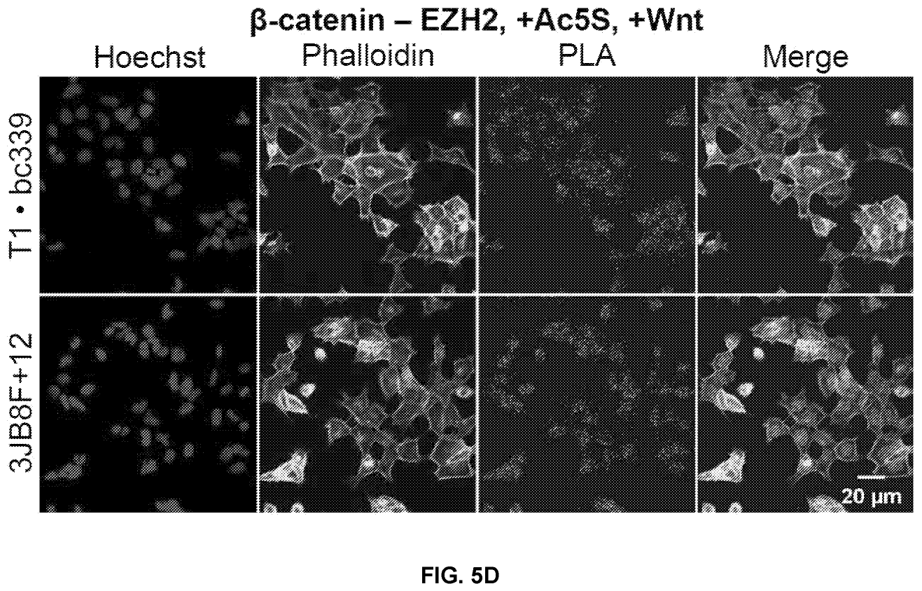

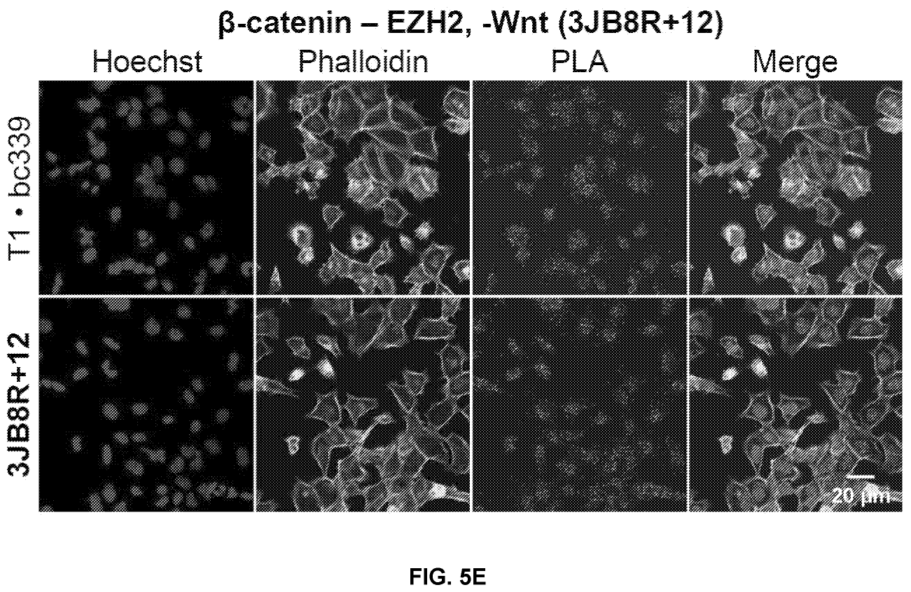

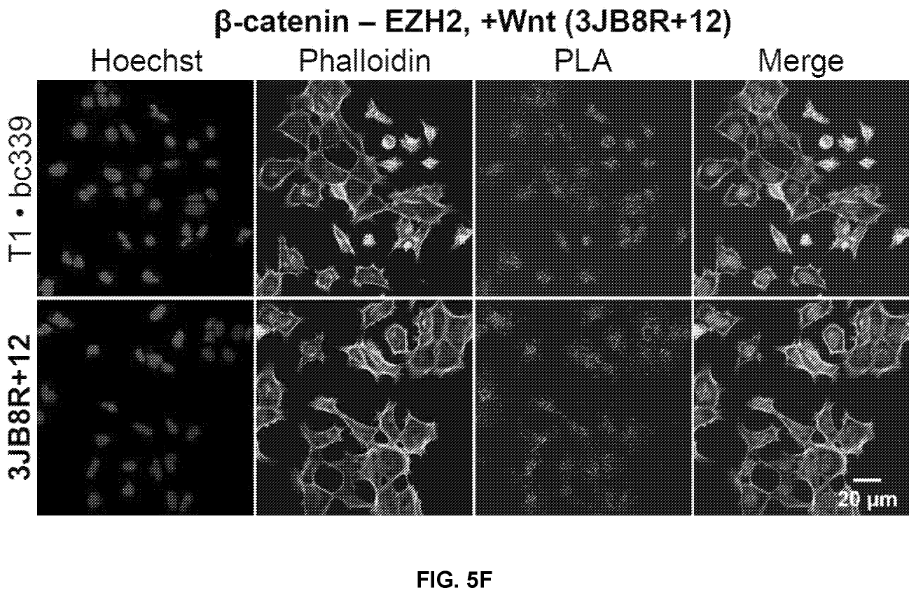

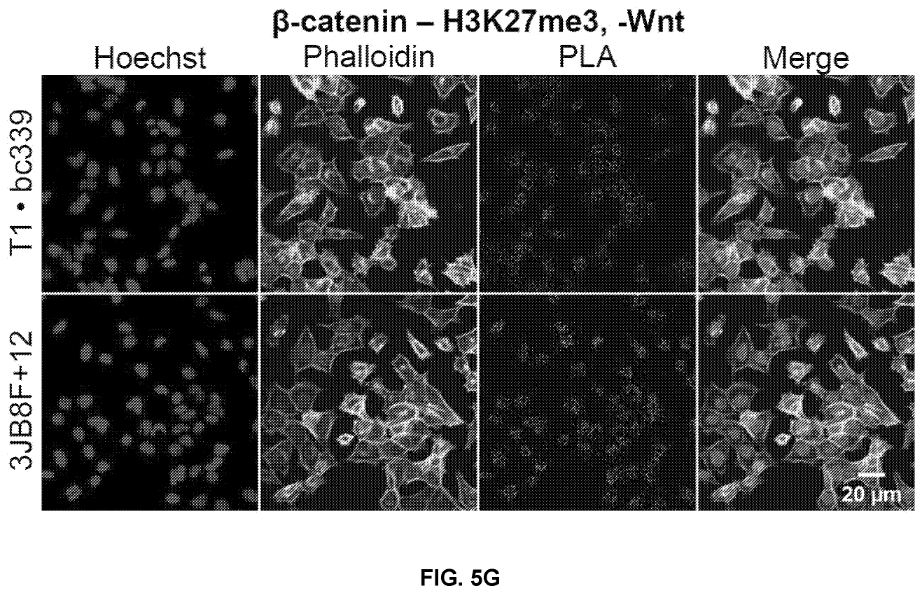

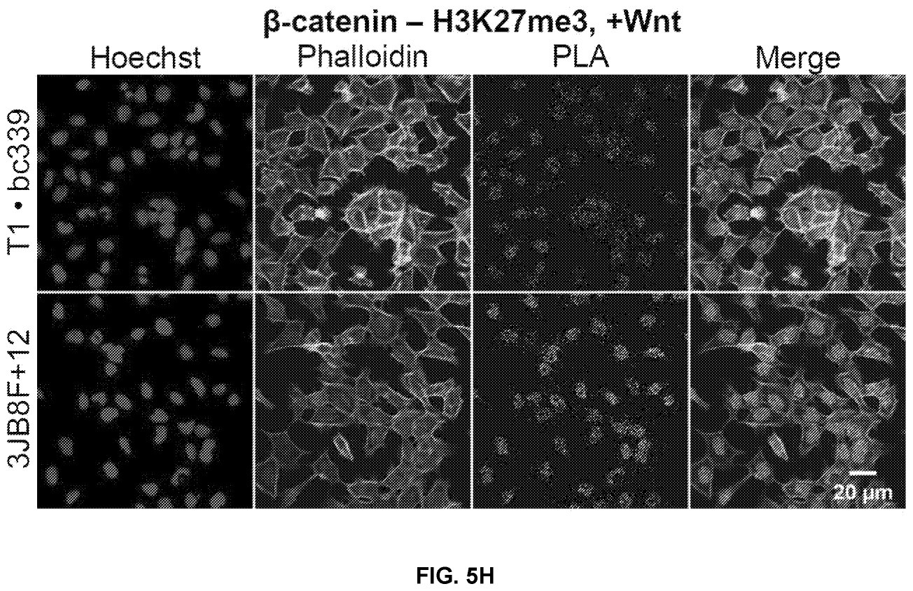

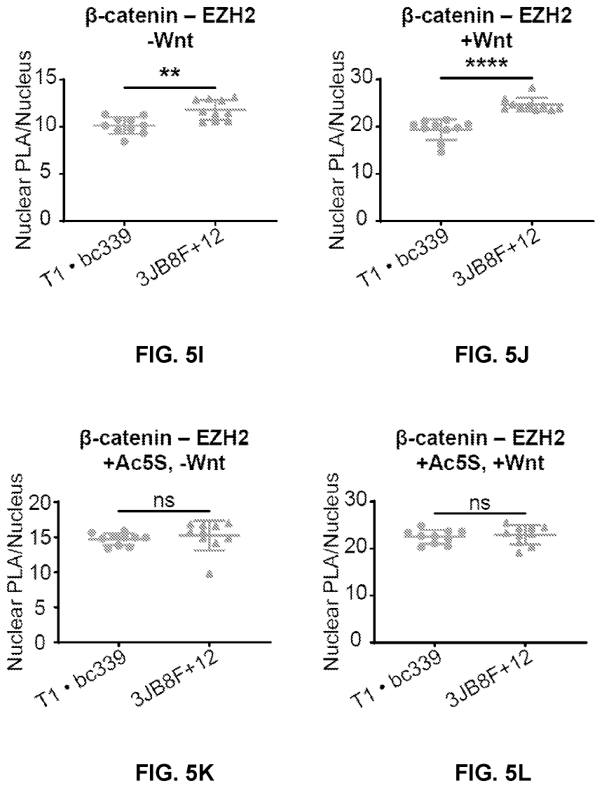

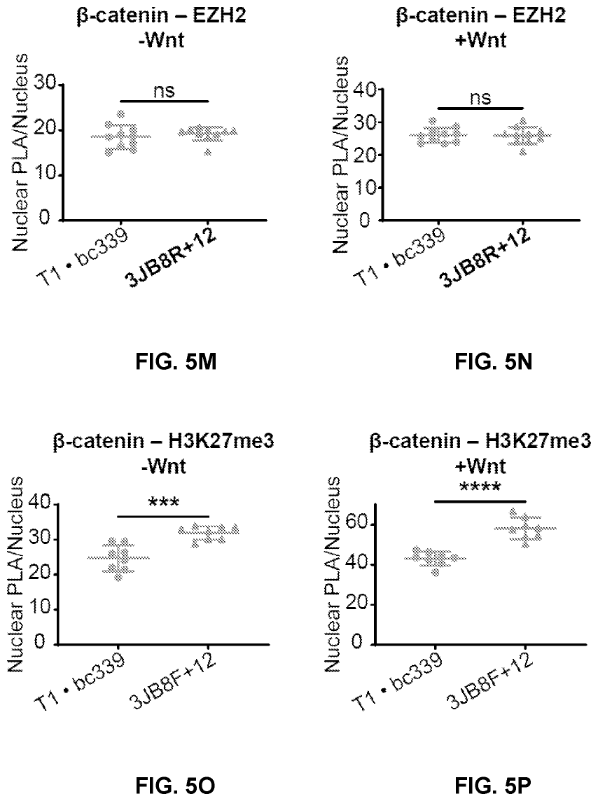

Enhancer of zeste homolog 2 (EZH2) is the catalytic subunit of the Polycomb Repressive Complex 2 (PRC2), which plays important roles in gene repression and during development (Margueron, R., et al. Nature 2011 469:343-349; Lavarone, E., et al. Nat Commun 2019 10:1679). It catalyzes the tri-methylation of Lys27 on Histone H3 (H3K27me3), which marks silent promoters (Barski, A., et al. Cell 2007 129:823-837). In PLA and Co-IP, the interaction between β-catenin and EZH2 was increased by 3JB8F+12, regardless of the Wnt signaling status (FIGS. 5A, 5B, 5I, 5J, 11M, 11N). This increase was prevented by inhibition of OGT (FIGS. 5C, 5D, 5K, 5L). Furthermore, 3JB8R+12, the DS aptamer that does not change O-GlcNAc levels on β-catenin, had no effect on this interaction (FIGS. 5E, 5F, 5M, 5N). Proximity between 3-catenin and H3K27me3 was also increased by 3JB8F+12 (FIGS. 5G, 5H, 5O, 5P). It was concluded that O-GlcNAcylation of β-catenin promotes its interaction with EZH2.

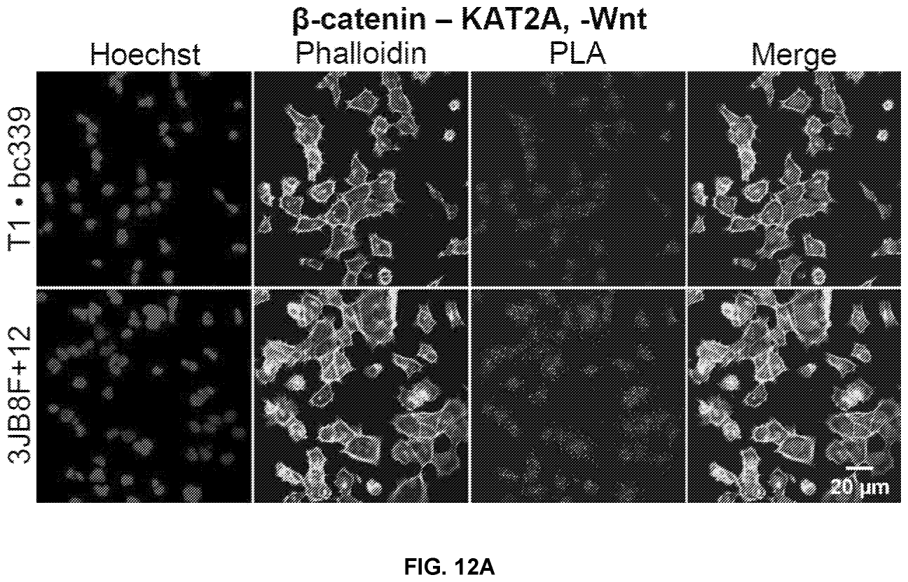

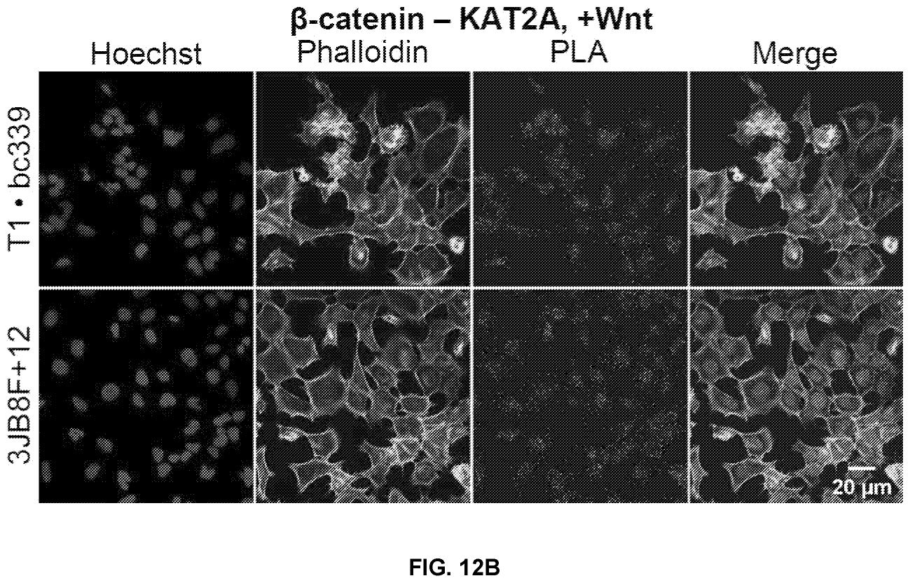

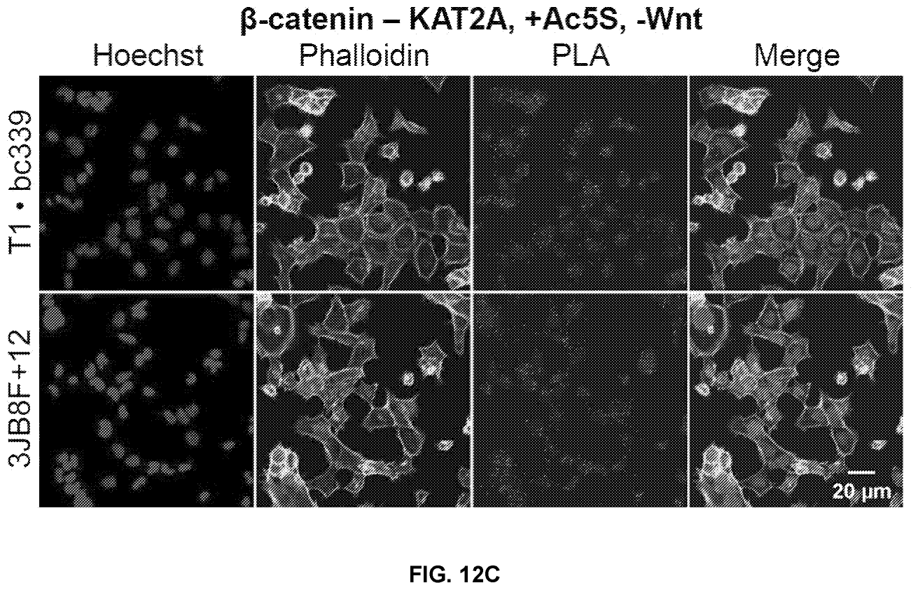

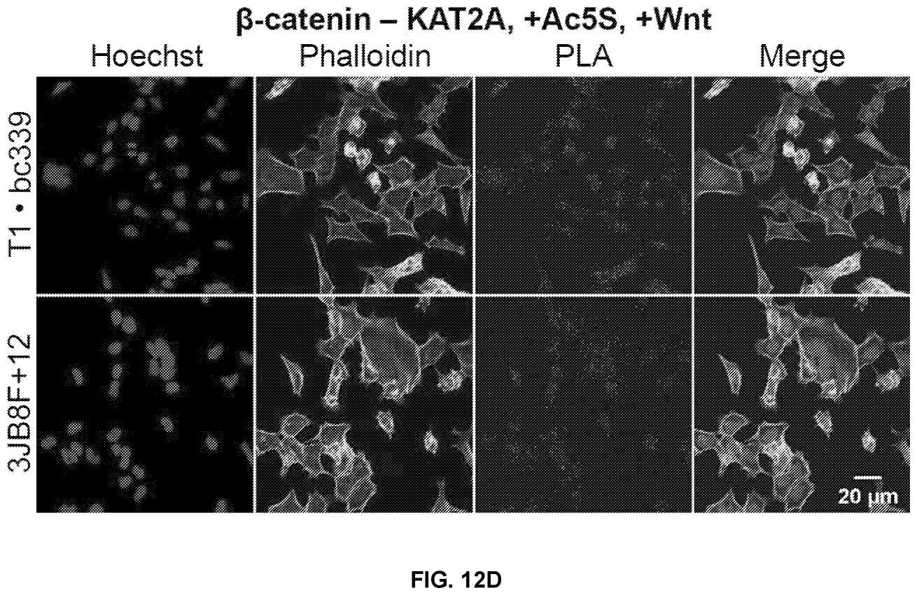

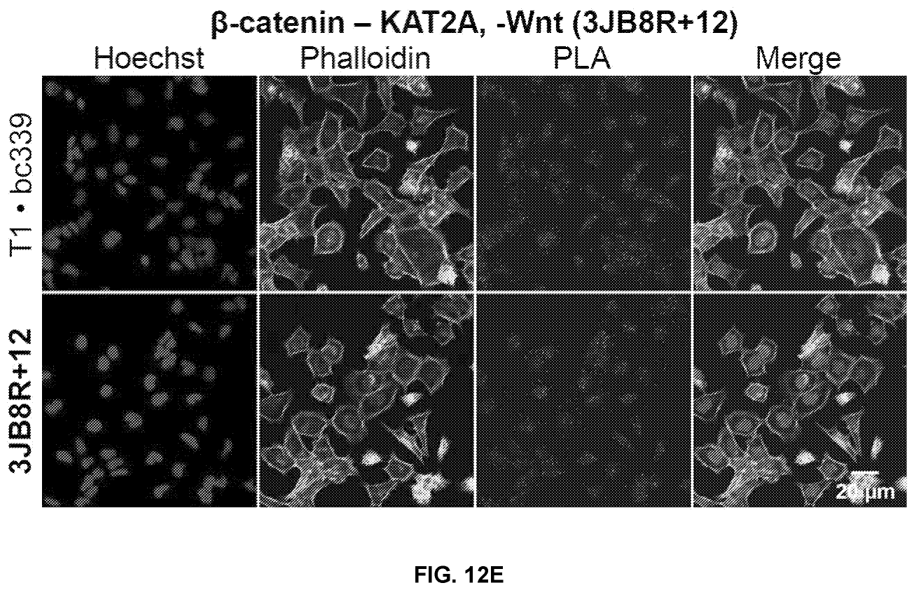

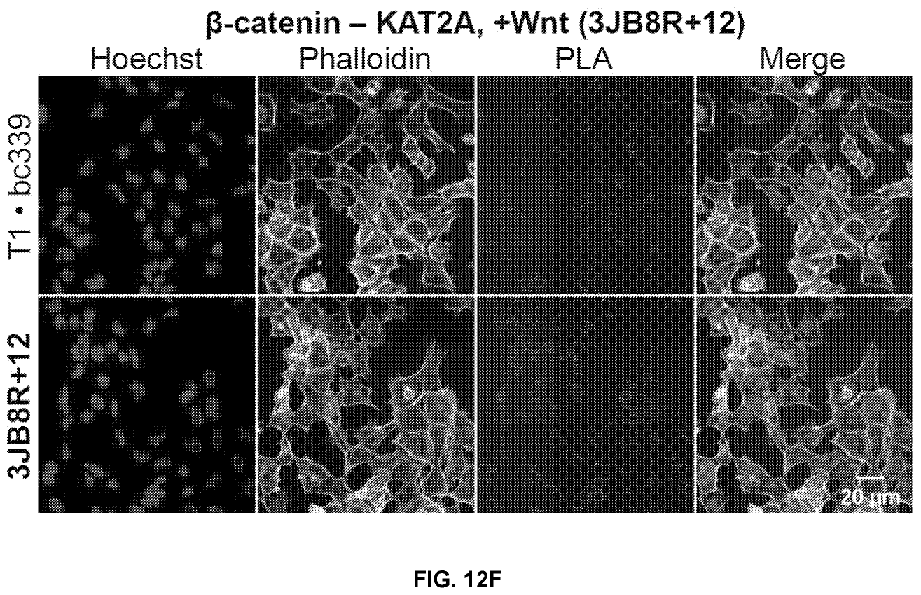

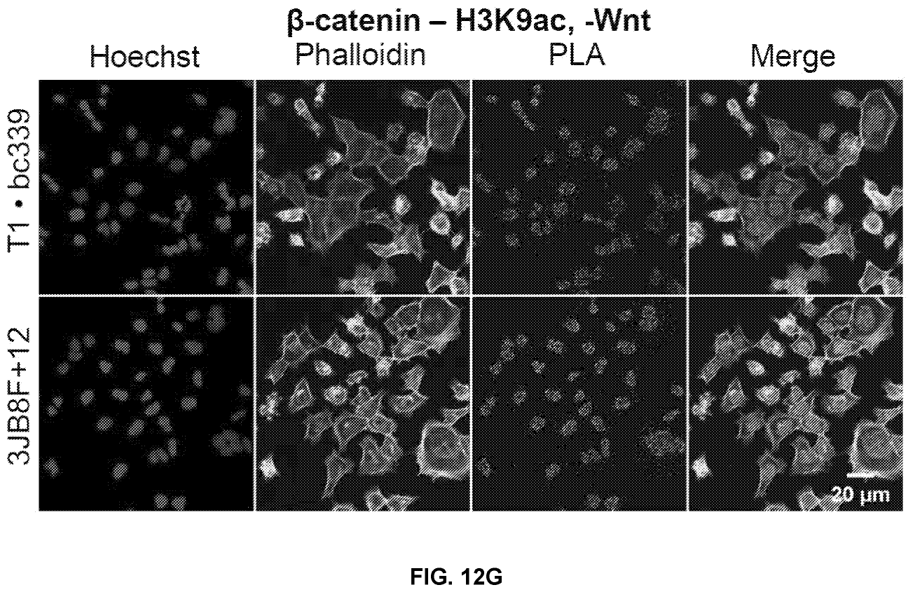

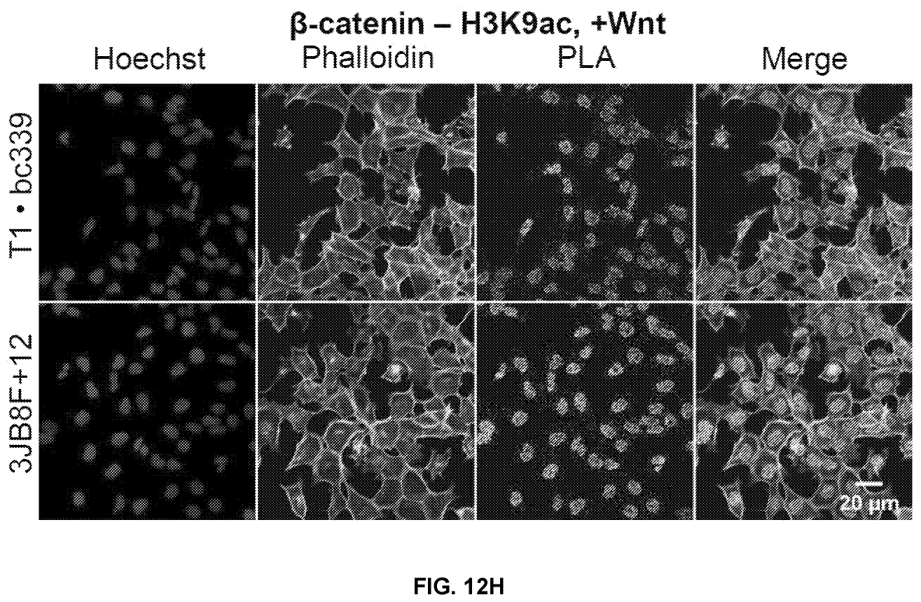

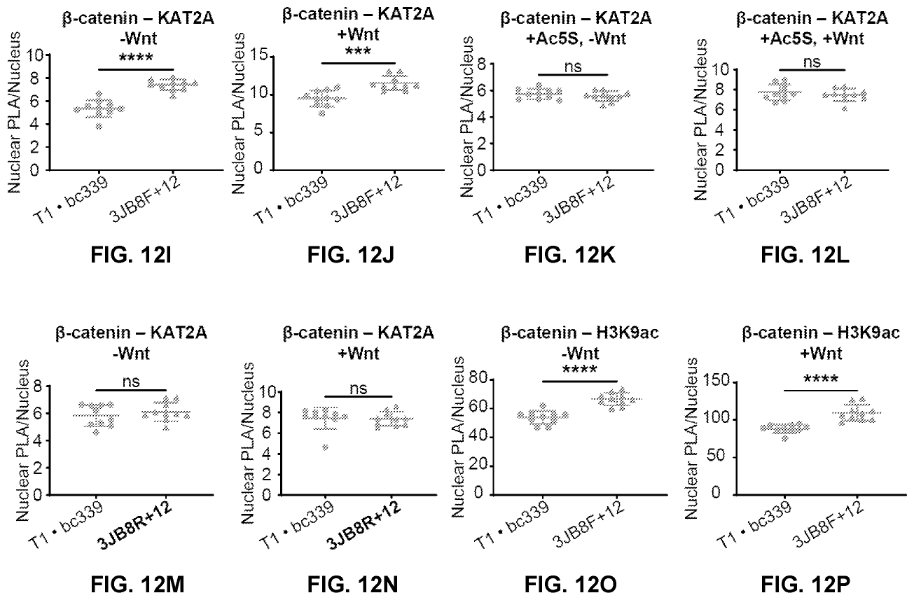

KAT2A (GCN5) is a histone acetyltransferase that acetylates the Lys9 of histone H3 (H3K9ac). H3K9ac correlates with actively expressed genes (Karmodiya, K., et al. BMC Genomics 2012 13:424). In PLA, 3JB8F+12 increased the interaction between 3-catenin and KAT2A in spite of the Wnt signaling status (FIGS. 12A, 12B, 12I, 12J). Inhibition of OGT prevented this change (FIGS. 12C, 12D, 12K, 12L). Expression of 3JB8R+12 had no effect on this interaction (FIGS. 12E, 12F, 12M, 12N). Moreover, 3JB8F+12 also increased the proximity between β-catenin and H3K9ac (FIGS. 12G, 12H, 12O, 12P). In conclusion, O-GlcNAc on β-catenin enhances its interaction with KAT2A.

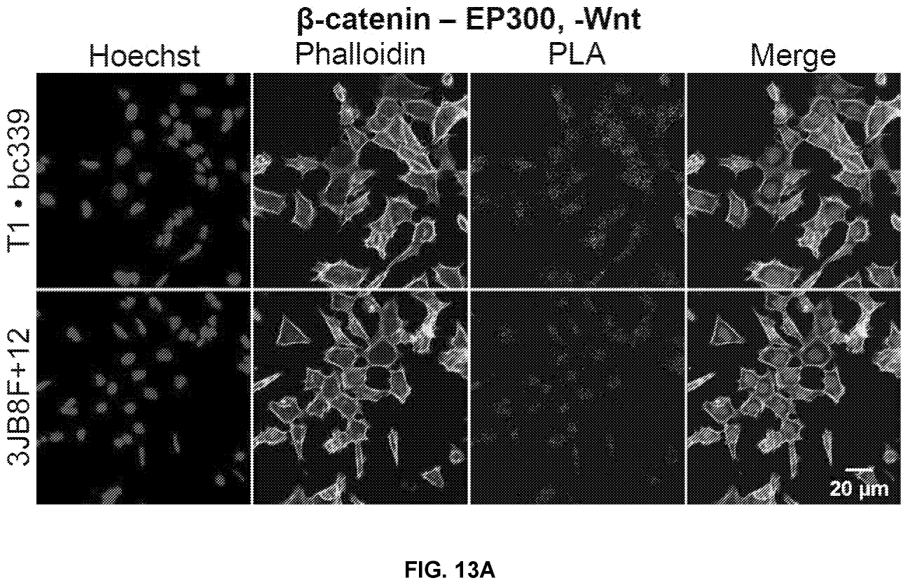

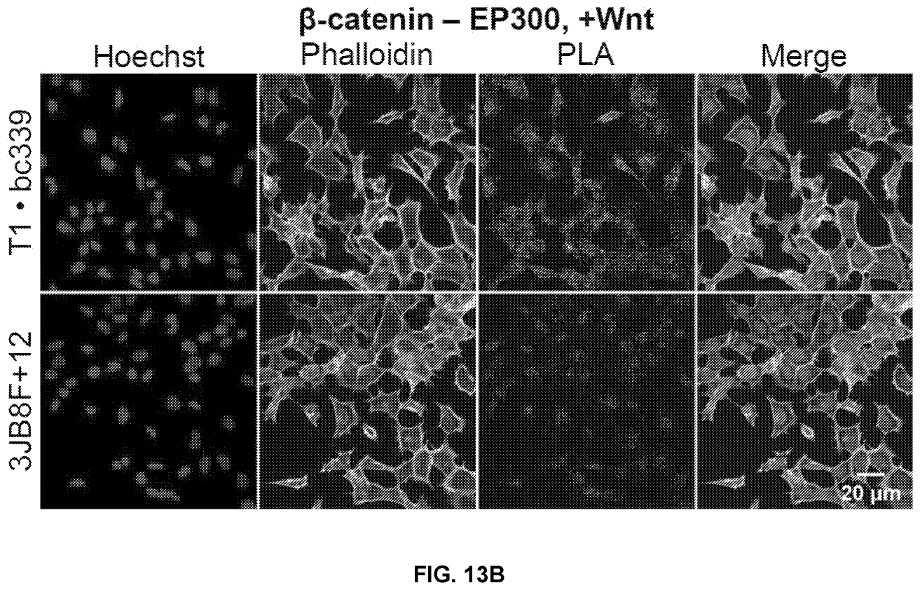

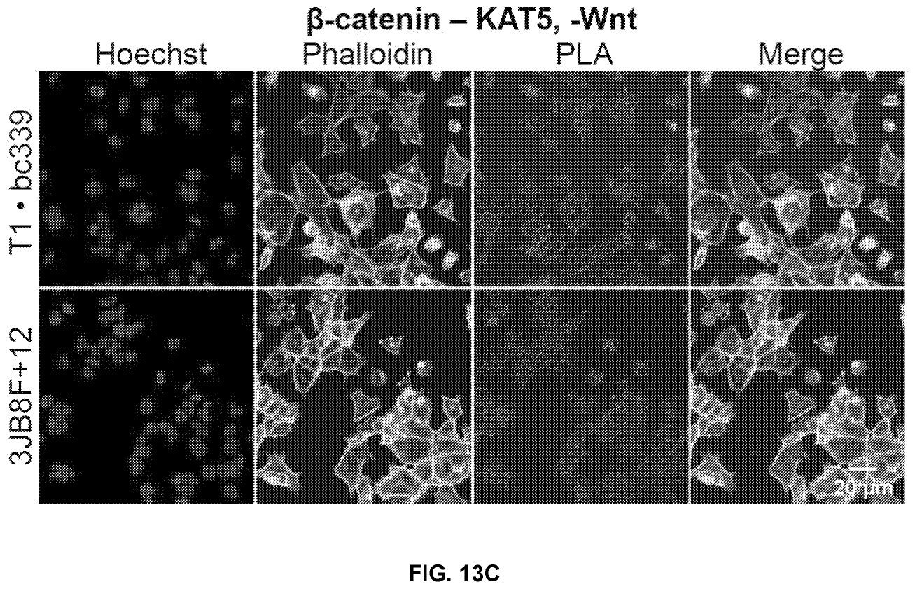

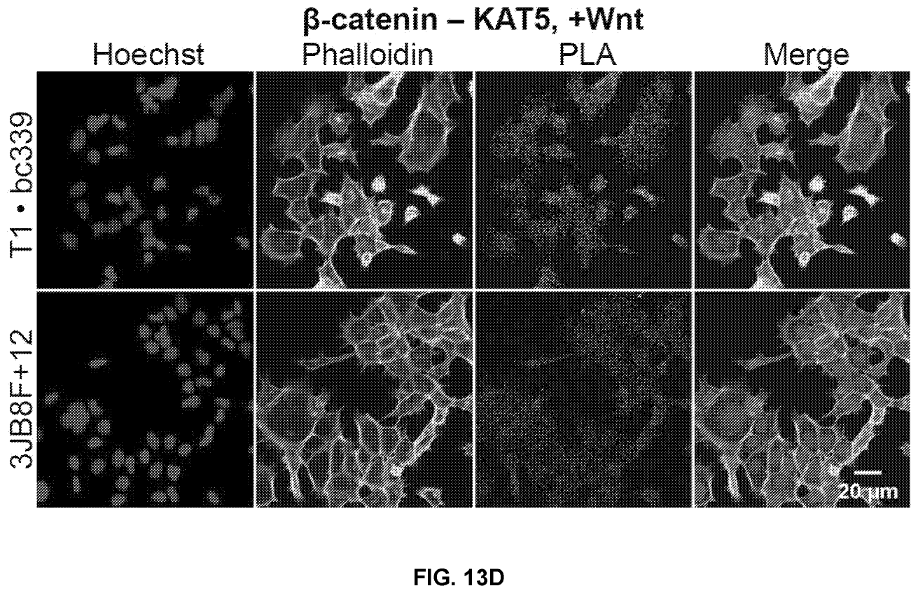

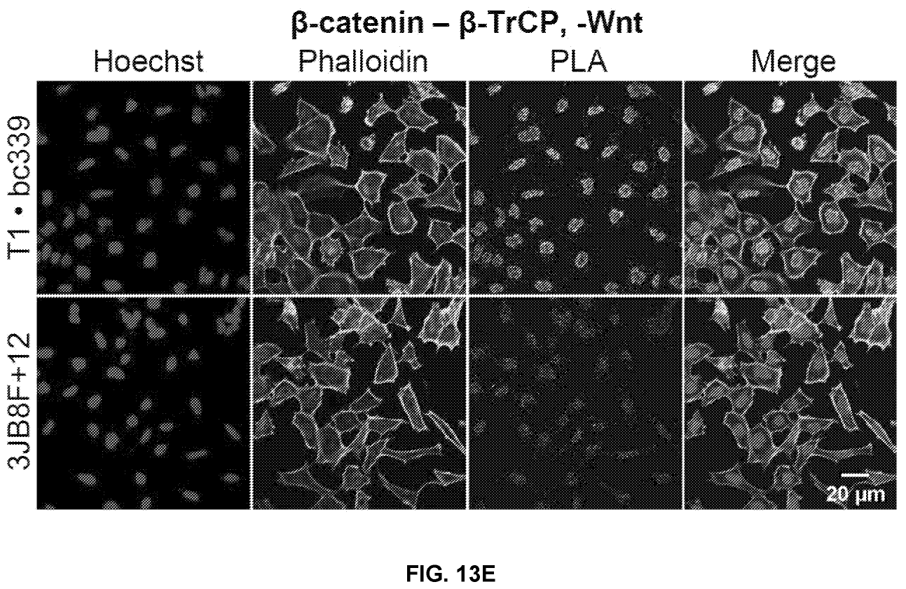

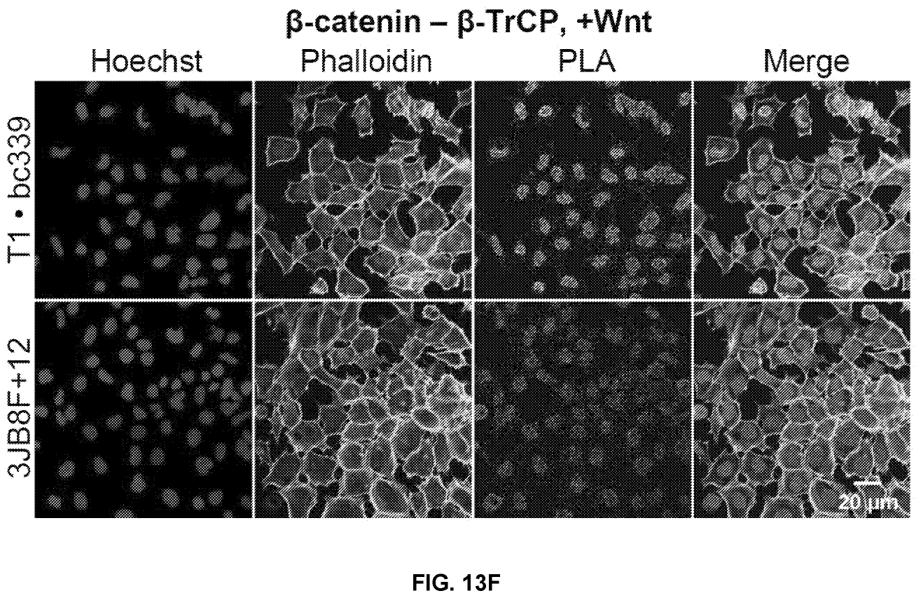

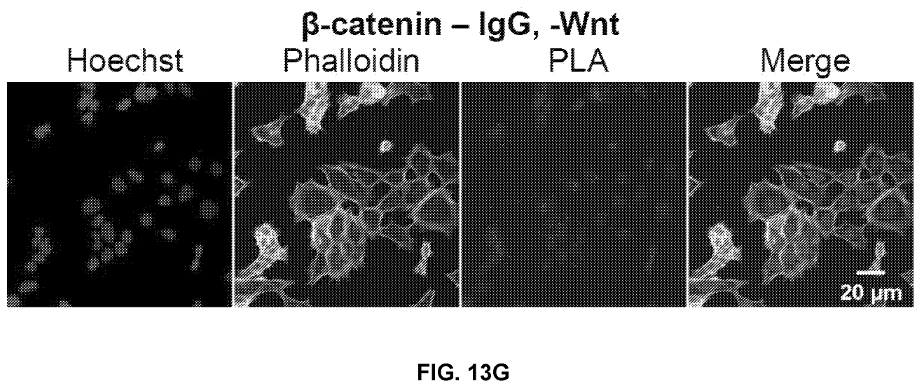

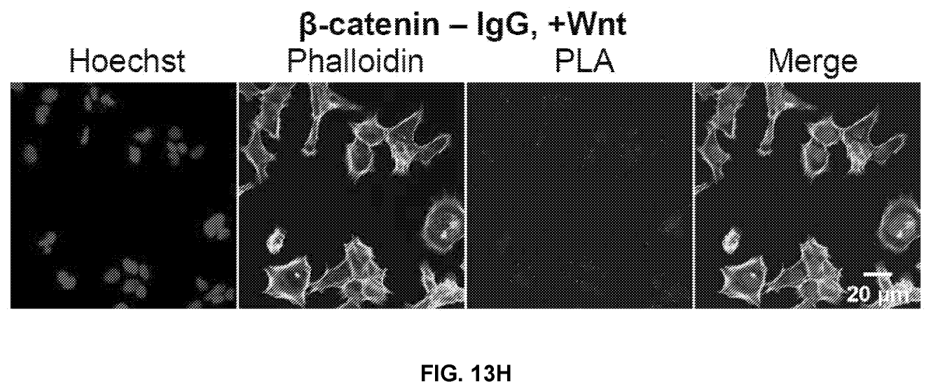

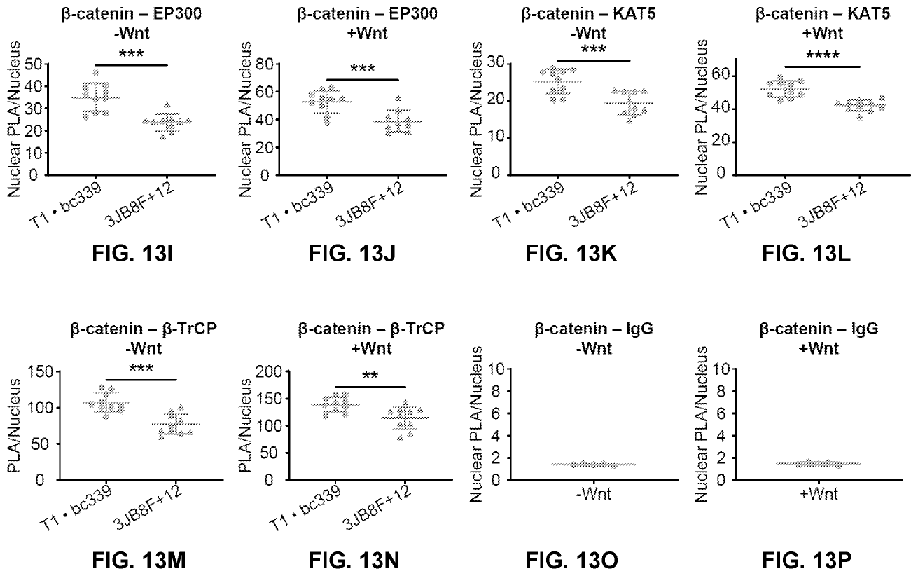

3JB8F+12 regulates the interactions between β-catenin and other histone modifiers. It significantly inhibited the interaction between β-catenin and EP300, an acetyltransferase and transcription coactivator (FIGS. 13A, 13B, 13I, 13J) (Li, J., et al. EMBO J 2007 26:2284-2294). It also impaired the interaction between β-catenin and KAT5 (TIP60), a lysine acetyltransferase that involves in the regulation of transcription, in DNA repair and in apoptosis (FIGS. 13C, 13D, 13K, 13L) (Bakshi, K., et al. Sci Rep 7:3635). Additionally, this DS aptamer inhibits the interaction between endogenous β-catenin and β-TrCP (FIGS. 13E, 13F, 13M, 13N), agreeing with our observation on GFP-β-catenin and β-TrCP (FIGS. 3A, 3B). Negative control experiments revealed good specificity of PLA experiments (FIGS. 13G, 13H, 13O, 13P).

### O-GlcNAc on β-Catenin Increases its Transcriptional Activity, Recruits EZH2 to Promoters and Shifts the Transcriptome

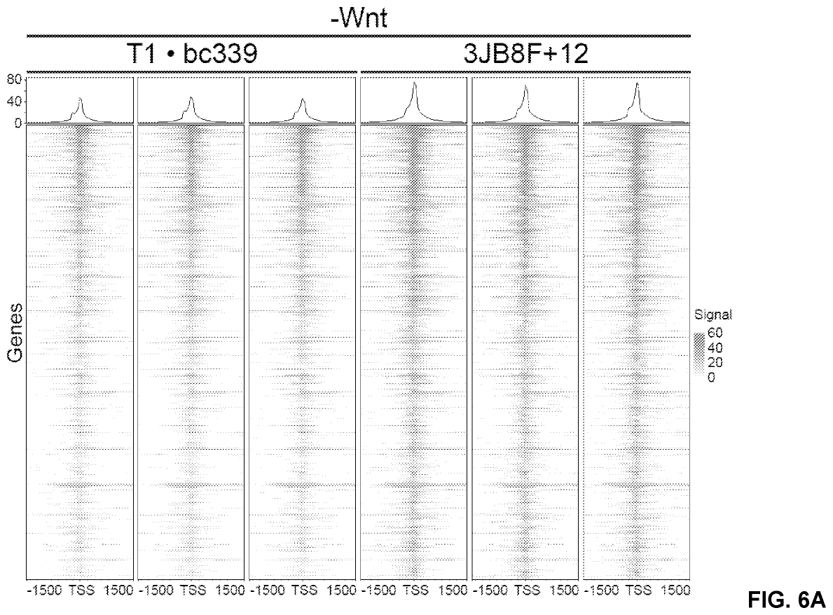

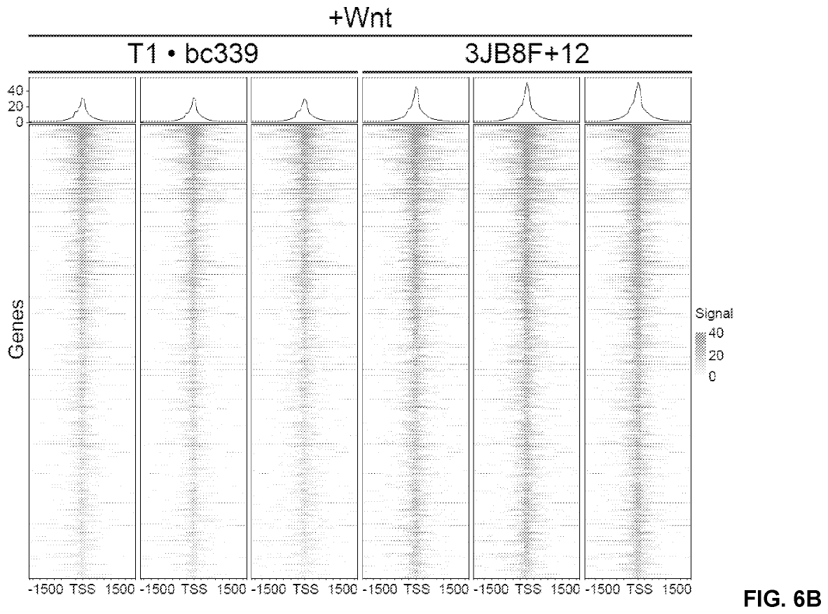

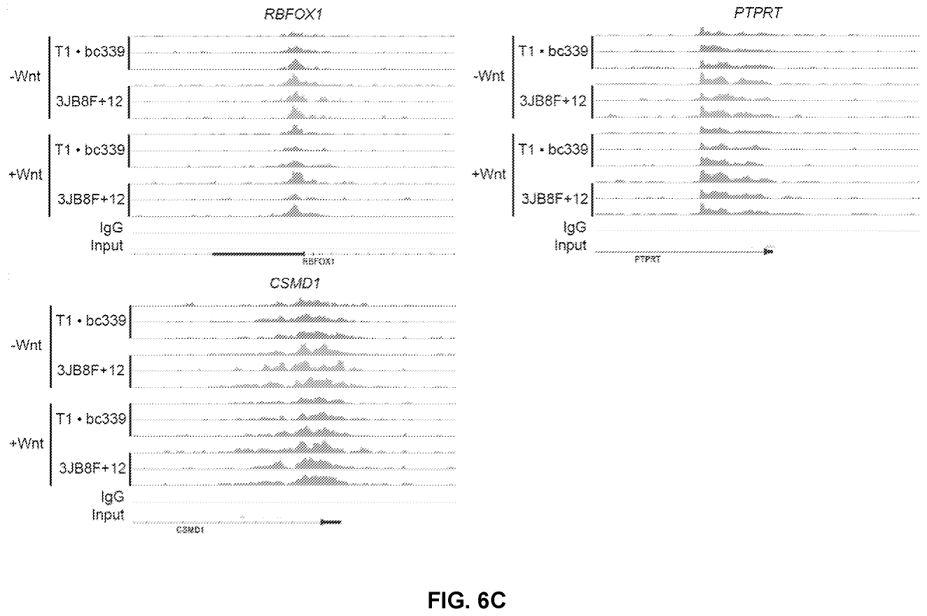

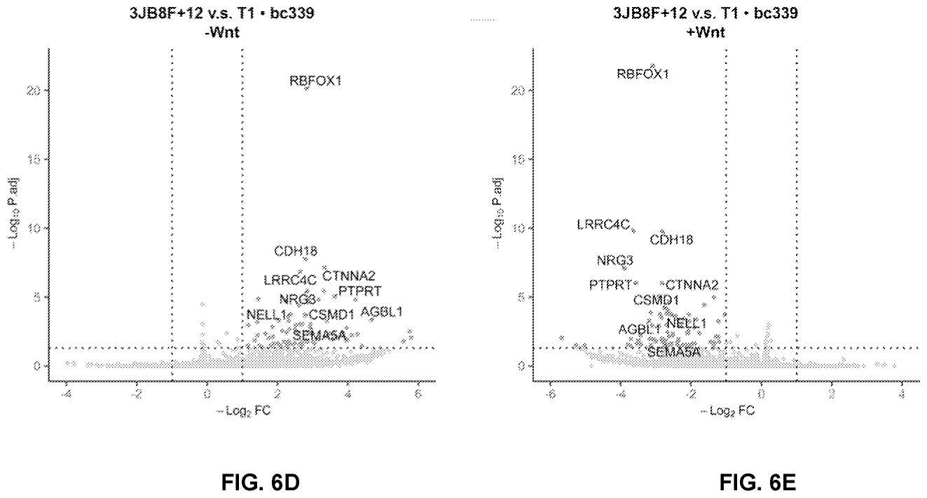

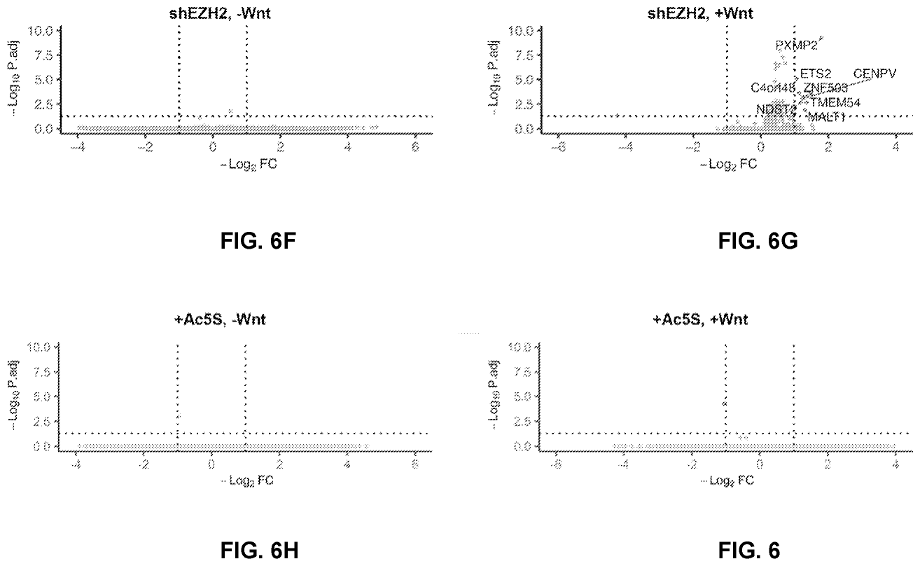

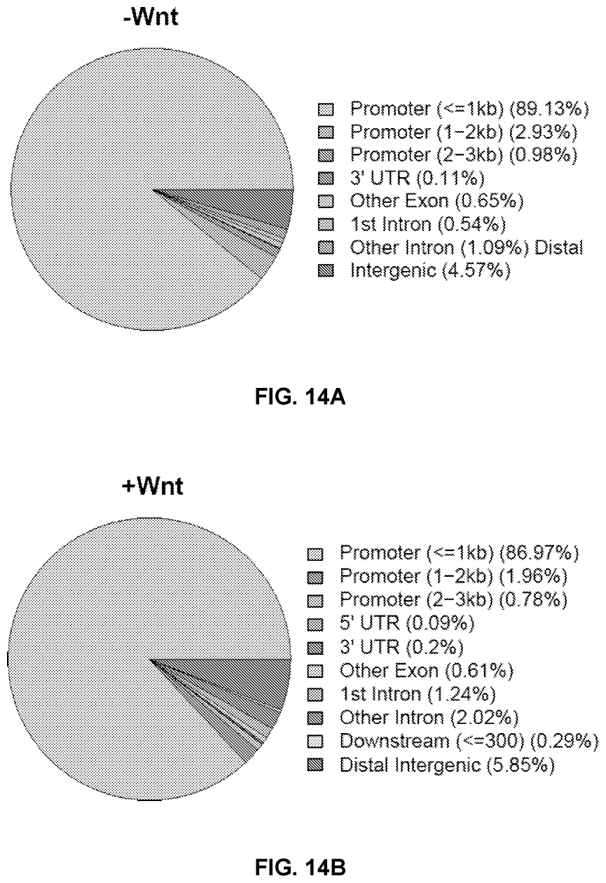

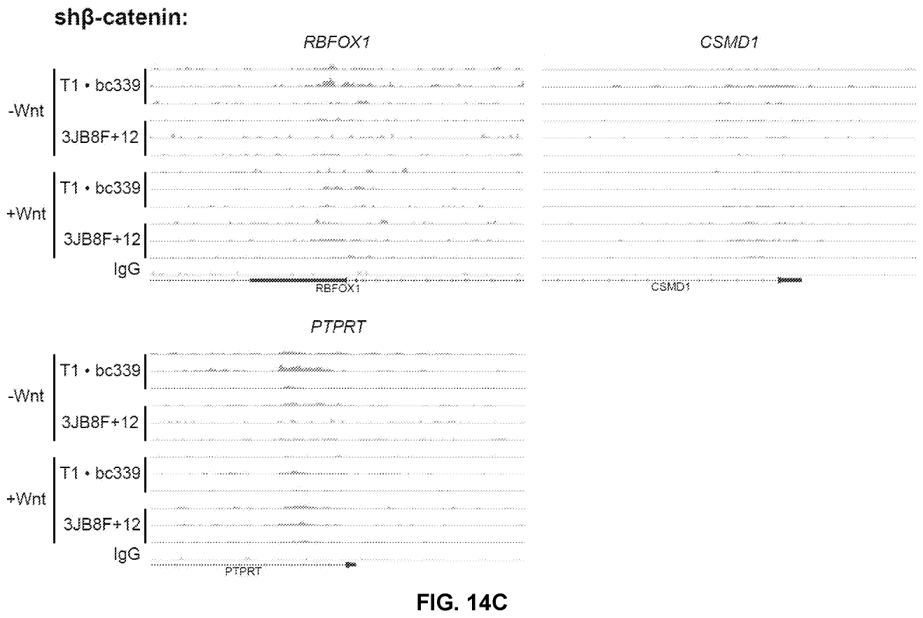

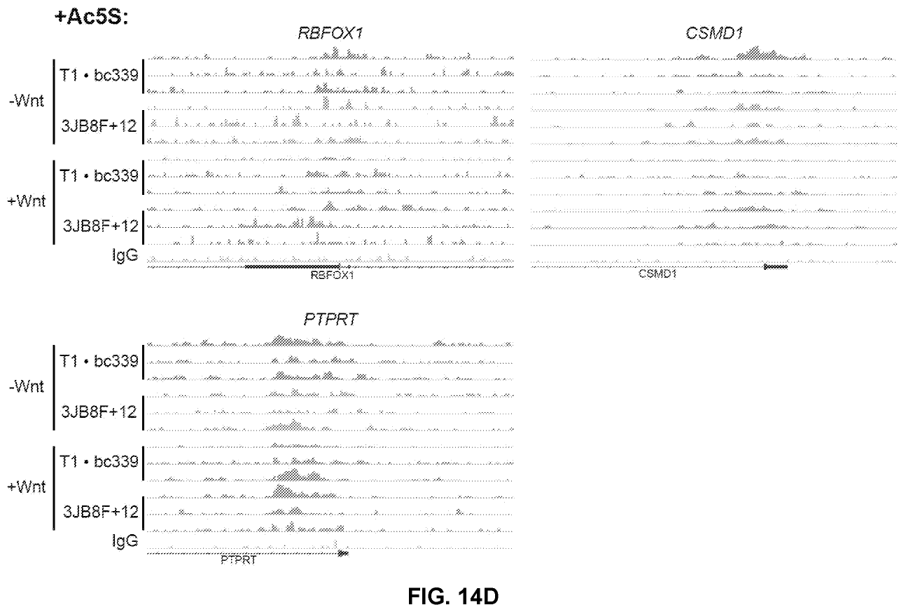

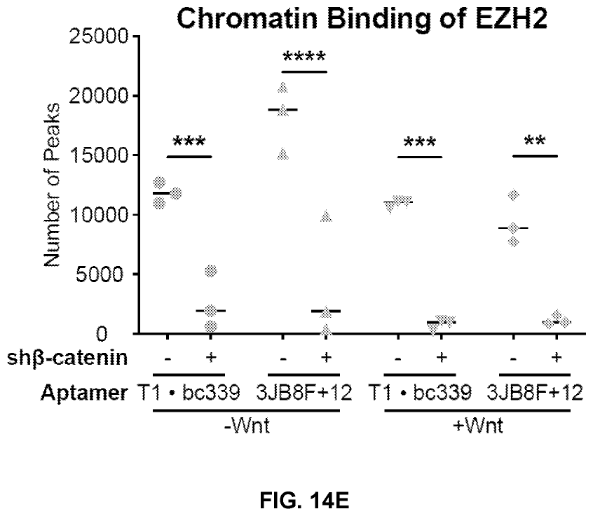

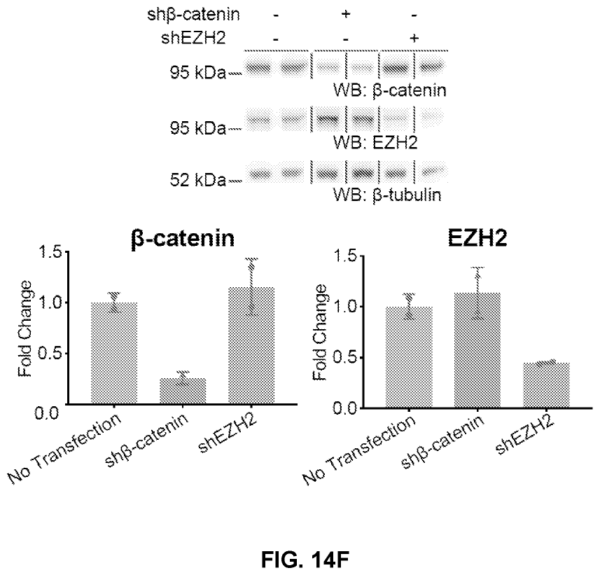

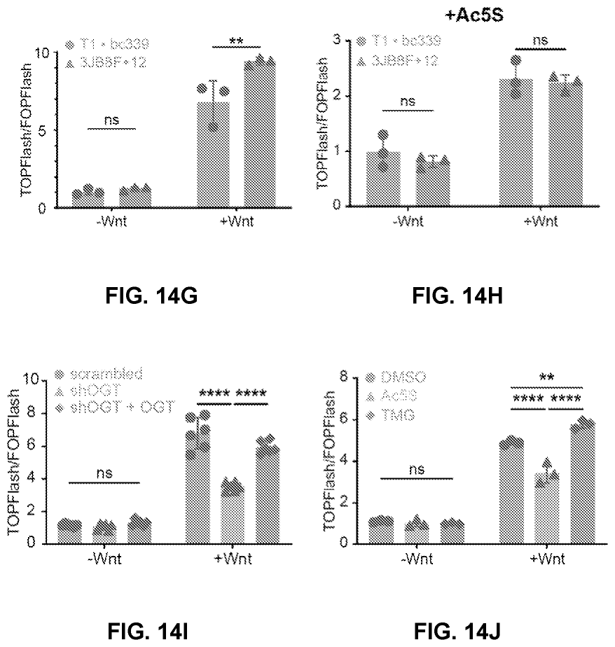

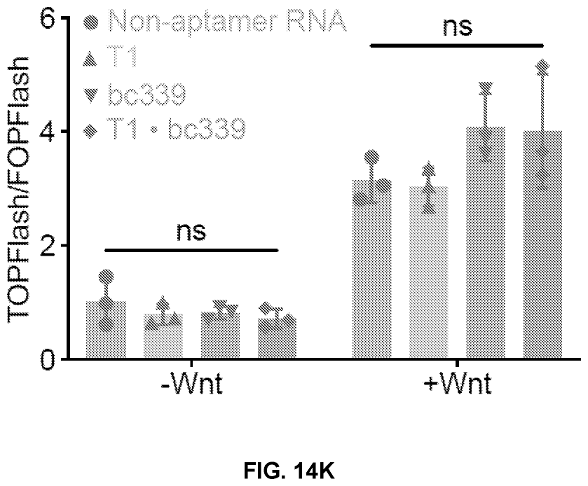

The next question was whether the enhanced interaction with β-catenin regulates the chromatin binding of EZH2. Cleavage Under Targets and Release Using Nuclease (CUT&RUN) was performed on HEK293T cells expressing the individual aptamers (T1 bc339) or the DS aptamer (3JB8F+12) (Skene, P. J., et al. Elife 2017 6; Meers, M. P., et al. Elife 2019a 8). CUT&RUN sequencing data revealed that 3JB8F+12 increased the binding of EZH2 on 923 and 3473 sites in the absence and presence of Wnt, respectively (FIGS. 6A to 6C). The differential binding sites predominantly localized on the promoter regions of the genome (FIGS. 14A and 14B). All differential binding events of EZH2 were prevented by knocking-down β-catenin, or by inhibiting OGT (FIGS. 14C, 4D, 14F), indicating they were specific to increased O-GlcNAcylation on β-catenin. In addition, knockdown of β-catenin drastically impaired the promoter binding of EZH2 without affecting its abundance (FIGS. 14E, 14F), suggesting that β-catenin plays a critical role in recruiting EZH2 to the chromatin. Given that O-GlcNAc on β-catenin enhances its interaction with EZH2 (FIGS. 5, 14M, 14N), it was conclude that the strengthened interaction recruits EZH2 to promoters.

Also asked was whether increased O-GlcNAcylation of β-catenin impacts the transcriptome. RNA sequencing (RNA-Seq) was performed on HEK293T cells expressing either the individual (T1·bc339) or the DS (3JB8F+12) aptamers, in the absence of presence of Wnt stimulation. Surprisingly, the DS aptamer altered the transcriptome in opposite ways depending on the status of Wnt signaling. In the absence of Wnt, 3JB8F+12 promoted the expression of 95 genes. In contrast, when Wnt signaling was activated, 3JB8F+12 repressed the expression of 102 genes (FIGS. 6D, 6E; Table 3). These differential expression events were specific to elevated O-GlcNAc, as they were prevented by inhibiting OGT (FIGS. 6H, 6I). EZH2 is also required for these events, as its knockdown prevented the differential expression (FIGS. 6F, 6G, 14F). However, when EZH2 was knocked-down and Wnt was present, O-GlcNAcylation of β-catenin slightly enhanced the transcription of eight other genes (FIGS. 6G, 6I). This observation suggests that O-GlcNAc on β-catenin also has mild, EZH2-independent activating effects on transcription, which could be overwritten by its EZH2-dependent repressive effects.

Considering that EZH2 is associated with PRC2 that is a transcription repressor, the increased promoter binding of EZH2 may function in the repression of genes caused by O-GlcNAcylation of β-catenin when Wnt signaling was activated. However, the mechanism underlying the activation of genes without Wnt awaits further investigation. As EZH2 was reported to activate the androgen receptor gene independently of PRC2 and its methyltransferase activity (Kim, J., et al. Cell Rep 2018 25:808-2820), it is possible that EZH2 activated the genes we observed in the same way. It is also possible that O-GlcNAc on β-catenin recruited some transcription activators to the chromatin when Wnt was absent, which we have not identified in this research.

The aptamers were used to study how O-GlcNAc on β-catenin regulates its transcriptional activity in Wnt signaling. In TOPFlash assays (Table 6), DS aptamer elevated the activity of β-catenin in response to Wnt (FIG. 14G), and this elevation required the activity of OGT (FIG. 14H). These results agree with the observations when the O-GlcNAc modifying enzymes was inhibited with genetic or chemical approaches. Knockdown of OGT considerably impaired the activity of β-catenin, which was restored by rescuing this enzyme (FIG. 14I). Reducing global O-GlcNAcylation with the OGT inhibitor (Ac5S) impaired the activity of β-catenin, but elevating global 0-GlcNAcylation with the OGA inhibitor (TMG) further activated it (FIG. 14J). In a control experiment, neither single or double individual aptamers (T1, bc339, T1·bc339) affected the activity of β-catenin. In conclusion, O-GlcNAc on β-catenin increases its transcriptional activity. However, the luciferase reporter in TOPFlash assay was encoded on a plasmid and driven by an artificial promoter that contains seven TCF/LEF binding sites, its expression lacks the epigenetic regulations comparing to genes on the chromatin, but was highly sensitive to β-catenin. Thus, it measures the activity of β-catenin at high sensitivity, but the epigenetic regulations in a cell are not represented in this assay.

In conclusion, O-GlcNAc on β-catenin strengthens its interaction with EZH2 and recruits EZH2 to the promoters. Globally, it regulates the transcriptome in two ways: it activates gene expression in the absence of Wnt but represses gene expression when Wnt signaling is on. O-GlcNAc also increases the transcriptional activity of β-catenin in response to Wnt.

### Inducible Regulation of O-GlcNAcylation by Coupling Dual-Specificity Aptamers to Riboswitches or an Inducible Expression System

DS aptamers allow for regulation of O-GlcNAcylation on a single protein and to study protein-specific effects of this PTM. However, there is a concern about this method: will the normal functions of OGT and substrate proteins be interfered, if DS aptamers remain bound to them? This concern is partly solved by the fact that the individual and dual-specificity aptamers are short-lived (FIGS. 1J, 2B). Nevertheless, an inducible mechanism that forces the aptamers to dissociate from their targets would be ideal.

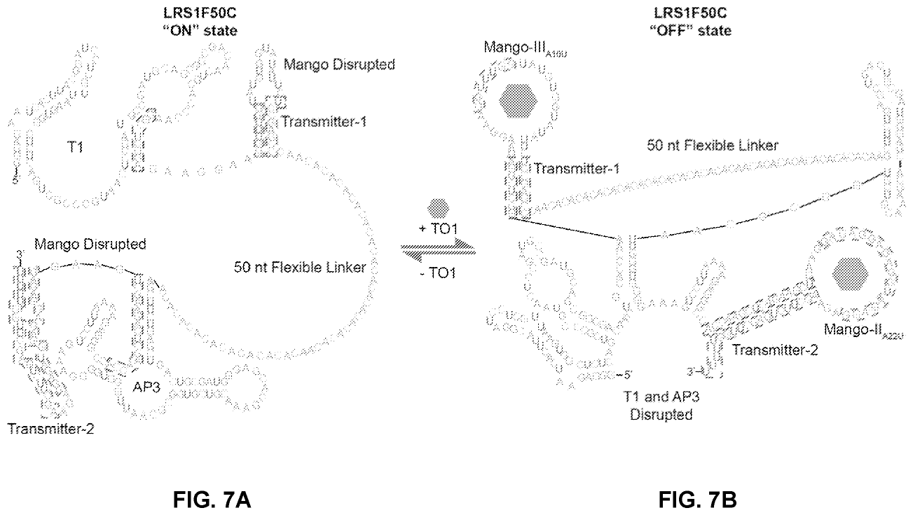

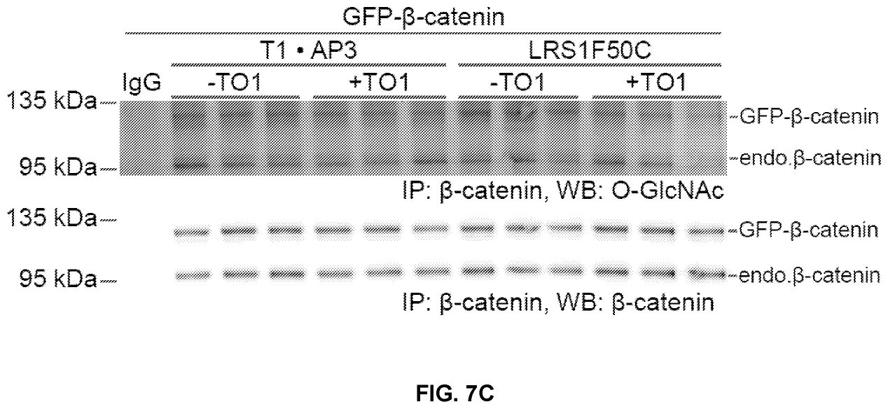

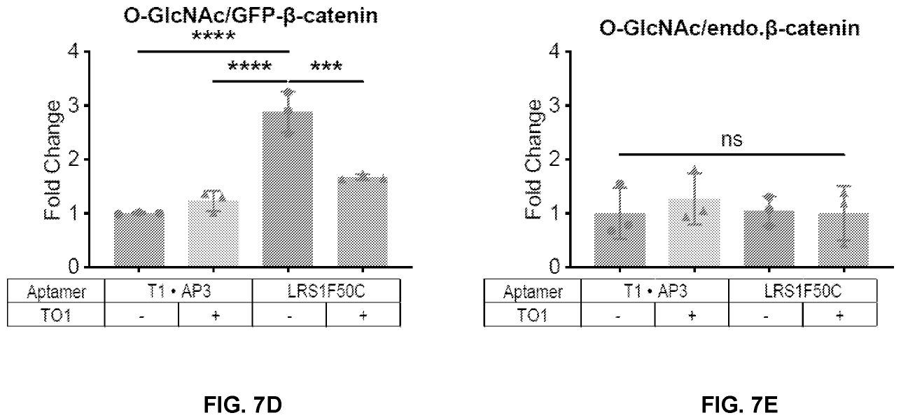

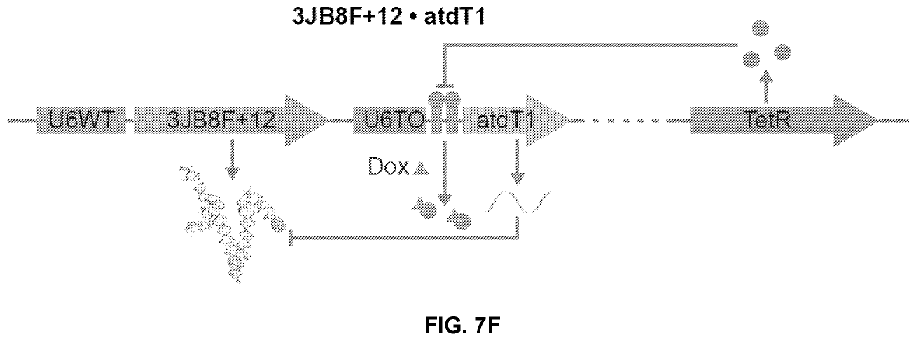

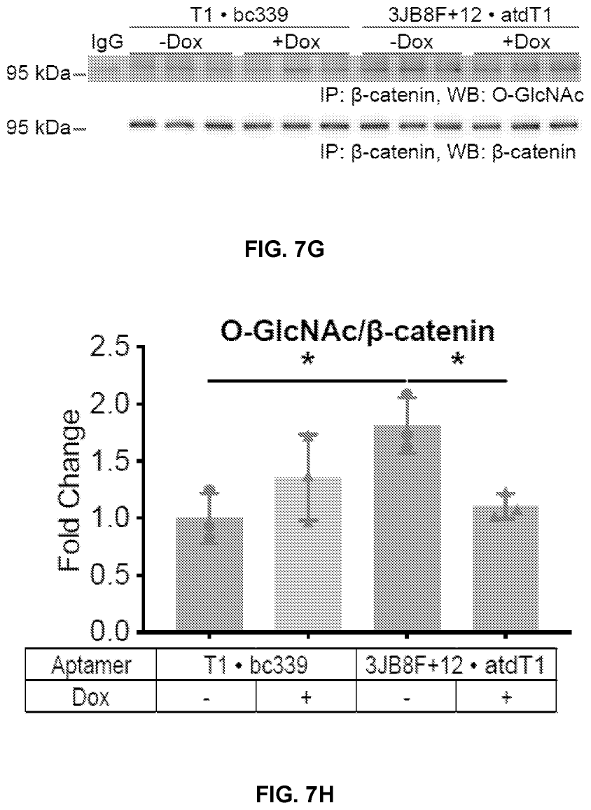

Inducible DS aptamers were designed by incorporating riboswitch elements. Riboswitches are naturally derived or artificially designed RNA that change their conformations upon binding of ligands (Serganov, A., et al. Cell 2013 152:17-24; Sherlock, M. E., et al. RNA 2020 26:675-693). Riboswitches usually contain three functional parts: a sensor that is a ligand-binding aptamer; an actuator that carries out downstream effects; and a transmitter sequence that couples the two (Win, M. N., et al. Science 2008 322:456-460). In this design, two sets of riboswitch mechanisms were incorporated into the DS aptamer NL1F50. In the newly generated inducible DS aptamer LRS1F50C (FIGS. 7A, 7B; Table 1), the fluorogenic “Mango” aptamers were employed as the sensors (Trachman, R. J., et al. Biochemistry 2018 57:3544-3548; Trachman, R. J., et al. Nat Chem Biol 2019 15:472-479). TO1-biotin (TO1), the ligand of the Mango aptamers, is a non-toxic, membrane permeable small molecule that binds the Mango with low nanomolar affinities (Dolgosheina, E. V., et al. ACS Chem Biol 2014 9:2412-2420). The T1 and AP3 aptamers served as the actuators, since it was their binding that the riboswitches aimed to regulate. The transmitter elements were designed so that they bind the core sequences of Mango in the absence of TO1, but switch to bind T1 and AP3 in the presence of TO1 (STAR Methods). As a result, addition of TO1 triggers the folding of Mango, which disrupts the folding of T1 and AP3 and “turns off” the function of the DS aptamer (FIGS. 7A, 7B). In cells, LRS1F50C increased O-GlcNAcylation of GFP-β-catenin by 3-fold without affecting that of endogenous β-catenin, and this increase was prevented by addition of TO1 (FIGS. 7C to 7E). These results showed the effectiveness of the riboswitch design, which confirmed the feasibility of inducible regulation of O-GlcNAcylation by riboswitch-coupled DS aptamers.

A Tet-On inducible expression system was also used to control the function of DS aptamers. In this system, 3JB8F+12 is constitutively expressed, driven by a wild-type U6 promoter (U6WT). An antidote RNA of T1 (atdT1) is expressed under an inducible promoter (U6TO), which combines a truncated U6 promoter with two Tet operator elements. Doxycycline (Dox) activates the expression of atdT1, which disrupts the folding of 3JB8F+12 and turns off its function (FIG. 7F). In cells, this system induced O-GlcNAc on endogenous β-catenin in the absence of Dox, and addition of Dox rendered it inactive (FIGS. 7G, 7H). Since aptamers are short-lived in cells (FIGS. 1J, 2B), this inducible expression system is expected to allow high time-resolution control of DS aptamers.

Taken together, using riboswitches or the Tet-On system, we showed the proof-of-principle that DS aptamers can be used for inducible regulation of protein-specific 0-GlcNAcylation. Further optimization on the design of the riboswitches and the U6TO promoter will help improve the specificities and dynamic ranges of these systems.

### DISCUSSION

RNA-based tools that target OGT to GFP-tagged or endogenous proteins were developed to induce protein-specific O-GlcNAcylation. Using these tools, it was found that O-GlcNAc stabilizes β-catenin, increases its transcriptional activity and regulates its interactions with multiple cellular proteins. Particularly, O-GlcNAc on β-catenin promotes its interaction with EZH2, recruits EZH2 to promoters and shifts the transcriptome in a Wnt-dependent manner. Finally, by coupling riboswitches or a Tet-On system to DS aptamers, we designed tools for inducible regulation of protein-specific O-GlcNAcylation.

O-GlcNAc Modifies Wnt Signaling by Regulating β-Catenin's Interactions with Other Proteins

The stability of β-catenin is negatively regulated by β-TrCP. This research reveals a mechanism that O-GlcNAc stabilizes β-catenin. O-GlcNAc inhibited the interaction between GFP-β-catenin and β-TrCP (FIGS. 3A, 3B), and stabilized GFP-β-catenin without competing its degradation-related phosphorylation (FIGS. 2E, 3C, 3D). The results were confirmed on endogenous β-catenin (FIGS. 11C, 11D, 13E, 13F, 13M, 13N). Considering that the Stokes radius of O-GlcNAc is five times that of a phosphate group (Hart, G. W. J Biol Chem 2019 294:2211-2231), O-GlcNAc could inhibit β-TrCP from recognizing phosphorylated β-catenin by steric hinderance. As a result, the phosphorylated β-catenin is no longer ubiquitinated. It remains bound to the destruction complex and saturates it. Then the newly synthesized, unphosphorylated β-catenin accumulates in cells.

It was also shown that O-GlcNAc on β-catenin promotes the transcriptional activity of this protein (FIGS. 14G to 14K). However, in TOPFlash assay the reporter was expressed from a plasmid, which lacked the epigenetic regulations on chromosomal genes. Its expression was also driven by an artificial promoter that is highly sensitive to β-catenin. Therefore, the increased activity of β-catenin was not reflected on the transcriptome (FIGS. 6D, 6E).

This research suggests novel O-GlcNAc sites on β-catenin (FIGS. 10A to 10J). It was also shown that O-GlcNAc on β-catenin regulates its interactions with KAT2A and EP300 (FIGS. 12, 3). Considering that β-catenin binds EP300 and KAT5 via its C-terminus (Hecht, A., et al. EMBO J 2000 19:1839-1850; Sierra, J., et al. Genes Dev 2006 20:586-600), and that O-GlcNAc interplays with phosphate on Ser675 (FIG. 3C), some of the unmapped sites may locate in this region. O-GlcNAc stabilized the GFP-β-catenin (4A) mutant (FIGS. 10B, 10D, 10F, 10H), considering that O-GlcNAc stabilizes β-catenin by inhibiting its interaction with β-TrCP (as discussed above), which recognizes phosphorylation on Ser33 and Ser37 (Liu, C., et al. Proc Natl Acad Sci USA 1999 96:6273-6278), some unidentified sites could be near this region. To map these sites will be important for investigating the site-specific effects of O-GlcNAc in the future.

### Critical Roles of the Linker

The linker domain, which critically affects the efficacy of DS aptamers, can adopt either flexible or folded structures. Our optimization of the folded linkers revealed that in a DS aptamer, both the distance between the two individual aptamers and their orientations are important factors. Folded linkers tend to increase the efficacy of DS aptamers (FIGS. 2C, 2D and 2J; 4D, 4F, 4I to 4L), probably because 1) they induce higher degrees of proximity than flexible linkers; and 2) the folding of these linkers is highly spontaneous (ΔG<0), which facilitates the folding of aptamers. Meanwhile, the rigidity of folded linkers seems to reduce the degrees of freedom of the bound proteins, so that some variations of these DS aptamers do not increase O-GlcNAcylation. Therefore, when using these DS aptamers to target a new protein, an optimization that covers 0-11 bp variations of the RNA helix (0-360° rotation) may be needed (Tanaka, Y., et al. Nucleic Acids Res 1999 27:949-955). Alternative choices are folded linkers with more flexibility, like the 4-way RNA junction we used in this study (FIGS. 2I, 2K, 2L, S21, 9J). In this linker, the H1 and H2 helices “swings”, which provides the bound proteins higher degrees of freedom.

Though the rigidity of folded linkers sometimes impairs the efficacy of DS aptamers, it could provide better site-selectivity when inducing O-GlcNAcylation. Comparing to NL1F30, 3JB1F+6 induced higher O-GlcNAcylation on the GFP-β-catenin mutant, but was less effective on stabilizing the protein (FIGS. 10A, 10B, 10E, 10F), suggesting that 3JB1F+6 has different site-selectivity. On a DS aptamer, the rigid RNA helix restricts the bound proteins so that only certain O-GlcNAc sites on the substrate are exposed to OGT, thus O-GlcNAcylation is preferably induced on these residues. Therefore, the substrate protein could be “rotated” gradually by introducing serial additions or deletions to the RNA helix where it resides, and O-GlcNAc on different sites can be induced.

### Limitations of the Study

IP-WB or Mass-shift assay was used to detect O-GlcNAc on β-catenin. An antibody of β-catenin was used to precipitate the protein in IP, or to detect its shifted bands in WB. However, most available antibodies recognize β-catenin on its N- or C-terminus, where O-GlcNAc sites are identified or suggested in this study. O-GlcNAc and PEG on the epitope could inhibit antibody recognition, thus the aptamer-induced O-GlcNAcylation in this study may be underestimated.

### Materials and Methods

### Cell Lines

The HEK293T cell line was obtained from ATCC (CRL-3216). The T1, NL1F50 and 3JB8F+12 cell lines were generated by transfecting the respective plasmids into HEK293T cells using TranslT-X2 (Mirus Bio, MIR 6000), and selected with 500 μg/mL hygromycin. The L Wnt-3A cell line was obtained from ATCC (CRL-2647).

### SELEX Library Construction

Systematic evolution of ligands by exponential enrichment (SELEX) was based on (Conrad, R. C., et al. Methods Enzymol 1996 267:336-367; Fitzwater, T., et al. Methods Enzymol 1996 267:275-301; Kenan, D. J., et al. Methods Mol Biol 1999 118:217-231). DNA oligos used in SELEX are listed in Table 5. The initial single-stranded DNA (ssDNA) libraries (L1-N60-ncs for OGT aptamers, L2-N50-ncs for 3-catenin aptamers) were synthesized by Integrated DNA Technologies (Coralville, IA, USA), using a customized recipe (A: 29%, C: 29%, G: 19%, T: 23%) for the random regions. ssDNA libraries were purified by Polyacrylamide gel electrophoresis (PAGE, Invitrogen EC6875BOX) and eluted from gel with Probe Elution Buffer (0.5 M Sodium Acetate, 1 mM EDTA, 0.2% SDS). Purified ssDNA was converted to double-stranded DNA (dsDNA) by annealing to L1-5′-T7-cs (for OGT aptamers) or L2-5′-T7-cs (for β-catenin aptamers) and Klenow extension (NEB M0210). RNA libraries were synthesized from PAGE-purified dsDNA libraries by in vitro transcription with T7 RNA polymerase (NEB M0251). RNA libraries were PAGE-purified, precipitated with isopropanol (Sigma-Aldrich 19516-500 mL), washed with 70% ethanol and dissolved in nuclease-free water.

### Partition and Amplification

To improve target specificity and to minimize PCR bias, “Toggle SELEX” and “RAPID-SELEX” was applied in the partition and amplification steps (White, R., et al. Mol Ther 2001 4:567-573; Szeto, K., et al. PLoS One 2013 8:e82667). Partition of library was performed in Aptamer Binding Buffer (ABB, 20 mM Tris-HCl, 150 mM NaCl, 5 mM MgCl₂, 0.05% Tween-20, pH 7.5) for OGT aptamers, or Intracellular Buffer (IB, 20 mM HEPES, 110 mM KCl, 10 mM NaCl, 0.4 mM MgCl₂, 5 mM KH₂PO₄, 0.05% Tween-20, pH 7.2) for β-catenin aptamers. Each RNA library and its desired target protein were incubated at 37° C. for 1 hr with rotation. The target proteins were either pre-immobilized on His-tag binding magnetic beads (MB, Invitrogen 10103D), or captured by nitrocellulose membrane disks (NC, Millipore HATF02500) after incubation. Unbound RNA sequences were washed away with 1 mL of ABB or IB. Elution of bound RNA was either by Proteinase K (Thermo Scientific E00492) digestion at 37° C. for 30 min followed by denaturation in 100 μL formamide (Thermo Scientific 17899) at 95° C. for 2 minutes, or with 300 mM imidazole. Eluted RNA was converted to cDNA using SuperScript IV Reverse Transcriptase (Invitrogen 18090010) and RT primers (L1-3′-const-ncs for OGT aptamers; L2-3′-const-ncs for β-catenin aptamers), then PCR amplified using Taq Polymerase (NEB M0273L) for 16 cycles. Counter selection (CS) was included in indicated rounds with 1 μg Hexa-His peptide (Abbiotec 350220) immobilized onto MB. In certain rounds of selection, eluted RNA was proceeded to the next selection cycles without amplification, in order to reduce amplification bias and time consumption (Szeto, K., et al. PLoS One 2013 8:e82667). Eluted RNA in the final cycles was converted to cDNA by RT-PCR (Invitrogen 18090010).

### Library Sequencing and Data Analysis

For OGT aptamers, the final DNA library was cloned into the pCR4-TOPO plasmid using the TOPO TA cloning kit (Invitrogen 450030) and transformed into Chemically Competent E. coli cells (TaKaRa 636766). 84 colonies were subjected to plasmid purification and Sanger-sequencing at the JHMI Synthesis & Sequencing Facility. Frequency of all resulted sequences were calculated, and the most represented sequence (T1) was chosen for validation.

For β-catenin aptamers, enriched DNA libraries from rounds 5 and 8 were submitted for Next-Generation Sequencing at the JHMI Transcriptomics and Deep Sequencing Core Facility, on an Illumina HiSeq 2500 sequencer.

Analysis of the sequencing data started from trimming of the sequencing adaptors and the constant flanking regions using cutadapt (Martin, M. EMBnet.journal 2011 17). Enrichment analysis was carried out using FASTAptamer (Alam, K. K., et al. Mol Ther Nucleic Acids 2015 4:e230). Frequencies of all sequences in each library were counted and clustered based on their similarity. Enrichment of each cluster was assessed across different SELEX rounds. The most enriched sequence (bc339) was chosen for validation.

### RNA Structure Prediction

Secondary structures of RNA were predicted using the RNAfold webserver (Lorenz, R., et al. Algorithms Mol Biol 2011 6:26) and drawn with StructureEditor in the RNAStructure 6.3 package (Reuter, J. S., et al. BMC Bioinformatics 2010 11:129). Tertiary structures were predicted using RNAComposer (Popenda, M., et al. Nucleic Acids Res 2012 40:e112; Antczak, M., et al. Acta Biochim Pol 2016 63:737-744), refined by QRNAS (Stasiewicz, J., et al. BMC Struct Biol 2019 19:5), and visualized with UCSF ChimeraX (Pettersen, E. F., et al. Protein Sci 2021 30:70-82).

### Surface Plasmon Resonance (SPR)

For SPR, Multi Cycle Kinetics was performed according to (Chang, A. L., et al. Methods Enzymol 2014 549:451-466) on a Biacore T100 (GE Healthcare/Cytiva) instrument at 25° C. In assay preparation, an amine-modified T24 DNA linker (NH2-T24) was immobilized to approximately 100 RU in both reference and test flow cells on a sensorchip PEG (GE Healthcare/Cytiva 29239810), using the Amine Coupling Kit (GE Healthcare/Cytiva BR100050). RNA of T1 or bc339 with a A28 tail on the 3′-end were diluted to 100 nM in ABB (T1) or IB+200 mM NaCl (bc339). Diluted RNA solutions were denatured at 65° C. for 5 min and re-folded at room temperature for 5 min. At the beginning of each cycle, the A28-tagged RNA solutions were injected into the test flow cell and captured by the immobilized T24 DNA linker. Protein analytes were diluted in ABB (OGT) or IB (β-catenin) and injected sequentially into both reference and test flow cells, at a rate of 30 μL/min. Concentrations tested are listed in the Figure legends. Each concentration was duplicated. At the end of each cycle, surface was regenerated with 25 mM of NaOH for 30 sec at 30 μL/min, which removed the captured RNA and protein. Data was analyzed using the Biacore Evaluation Software (GE Healthcare/Cytiva), where the overlaid sensorgrams were fitted into a 1:1 binding model. Curves were exported and replotted with ggplot2 for better illustration.

### Radio-Labeling of RNA

For end preparation, the terminal phosphate groups of RNA were removed by Antarctic Phosphatase (NEB M0289). Then the 5′-end of 200 μmol RNA was labeled with 10 μCi of [γ-32P]-ATP (American Radiolabeled Chemicals, ARP 0102F-250 μCi) using T4 Polynucleotide Kinase (NEB M0201). Labeled RNA was extracted with Acid-Phenol: Chloroform, pH4.5 (Invitrogen AM9720), precipitated with isopropanol, washed with 70% ethanol, and dissolved in nuclease-free water.

### Dot-Blot Assay

Radiolabeled RNA was diluted in ABB, denatured at 65° C. for 5 min and re-folded at room temperature for 5 min. Then 100 nM of RNA was incubated with multiple concentrations (see Figure legends) of purified ncOGT at 37° C. for 30 min. The volume of each assay was 50 μL. 1 μM yeast tRNA (Invitrogen AM7119) and 50 μg/mL Bovine Serum Albumin (BSA, NEB B9001S) were included as non-specific competitors. After incubation, the reactions were filtered sequentially through a nitrocellulose membrane (Amersham 10600042) and a Nylon membrane (Invitrogen AM10102), on a Bio-Dot Microfiltration Apparatus (Bio-Rad 170-6545). Membranes were washed with 100 μL ABB to remove unbound RNA. Washed membranes were air-dried and exposed to a film (Amersham 28906836) for 2 hr. The film was developed and scanned, and the image was analyzed with Fiji (Schindelin, J., et al. Nat Methods 2012 9:676-682). Quantified data was fitted into a Michaelis-Menten model using GraphPad Prism.

### Bacterial Expression of OGT-FL, TPR, β-Catenin and CpOGA

For OGT-FL, TPR and β-catenin: NiCo21 (DE3) Competent E. coli (NEB C2529H) transformed with a plasmid (pET24a-ncOGT-FL, pET24a-TPR or pET28a-β-catenin) was inoculated into 50 mL LB medium for overnight culture at 37° C., 250 rpm. On Day 2, the whole culture was transferred into 500 mL LB medium, and the growth was observed by measuring its OD₆₀₀ every 30 min. When the OD₆₀₀ reached 1.2, the whole culture was cooled on ice for 15 min. Protein expression was induced by adding 200 μM (OGT-FL and TPR) or 500 μM (β-catenin) IPTG (Invitrogen 15529019). Then the culture was continued at 16° C., 180 rpm for 24 hr. Cells were harvested by centrifugation at 4° C., 5000 rcf for 15 min and washed with cold PBS.

For CpOGA: NiCo21 (DE3) cells transformed with pGEX-6β-1-CpOGA (Rao, F. V., et al. EMBO J 25:1569-1578) was cultured and induced in the same way as above, except that protein expression was induced with 200 μM IPTG at 22° C., 250 rpm for 24 hr.

### Protein Purification

For OGT-FL, TPR and β-catenin: All steps in protein purification were performed at 4° C. Protease Inhibitor Cocktail (Roche 11873580001) was added into the Stock, Lysis, Wash and Elution Buffers below. Bacterial pellet was re-suspended in 10 mL Stock Buffer (50 mM Tris, 300 mM NaCl, pH 8.0). Cells were lysed by adding 10 mL Lysis Buffer (Stock Buffer+10 mM Imidazole, 1% Triton X-100, and 10 mg/ml Lysozyme (Thermo Scientific 89833)) and incubate on ice for 30 min. Lysate was homogenized by sonication on ice for 5 cycles of 12 sec ON, 30 sec OFF at power 4 (Fisher Scientific F550), centrifugation at 13,000 rcf for 15 min and collection of the supernatant. The supernatant was loaded onto a chromatography column packed with 2 mL Ni-NTA agarose slurry (Qiagen 30210) and passed the column twice under gravity flow. Then the column was washed with 45 mL Wash Buffer (Stock Buffer+30 mM Imidazole), and protein was eluted with 4 mL Elution Buffer (Stock Buffer+250 mM Imidazole). Elute was dialyzed three times against 4 L of Dialysis Buffer (20 mM Tris pH 7.5, 50 mM NaCl and 0.2 mM PMSF (Roche 10837091001) in a dialysis cassette (20K MWCO, Thermo Scientific 87735). The first and third dialysis were for 3 hr, and the second was for overnight. Dialyzed protein solution was concentrated with a Vivaspin protein concentrator (MWCO 30K, Cytiva 28932235). Protein concentration was measured by BCA assay (Thermo Scientific 23227). Purified proteins were stored at −80° C. for up to a year.

For CpOGA: Bacterial pellet was re-suspended and lysed in the same way as above, except that the Stock Buffer and Lysis Buffer were replaced with Buffer A (50 mM HEPES, 250 mM NaCl, pH7.5) and Lysis Buffer A (Buffer A+10 mg/mL lysozyme). The cleared lysate was loaded to an equilibrated column packed with 2 mL Glutathione Agarose Beads (Thermo Scientific 16100). The loaded column was capped and rotated for 2.5 hr at 4° C., to capture the GST-tagged CpOGA from lysate. Then lysate was allowed to pass through the column by gravity, and the beads was washed with 20 mL Buffer A. The CpOGA protein was eluted by cleaving the GST-tag. The cleavage was performed by adding 100 U PreScission Protease (GenScript Z02799-100U) in 5 mL Cleavage Buffer (50 mM Tris-HCl, 150 mM NaCl, 1 mM EDTA, 1 mM DTT, pH 7.0 at 25° C.) to the column, and rotating for overnight at 4° C. The elute was collected and dialyzed against 4 L of 1×TBS for overnight at 4° C. in a dialysis cassette (20K MWCO, Thermo Scientific 87735). The dialyzed protein solution was concentrated, quantified and stored as above.

### OGT Activity Assay (UDP-Glo)

Activity of OGT was measured using the UDP-Glo Glycosyltransferase Assay kit (Promega V6971), following the manufacturer's protocol. Assays were performed on a white 96-well half-area plate (Corning CLS3992-25EA). Each reaction contains 0.625 ng OGT, 50 μM CKII peptide substrate (PGGSTPVSSANMM, JHMI Synthesis & Sequencing Facility), 10 μM yeast tRNA and various concentrations (see Figure legends) of RNA aptamer in 12.5 μL ABB. Reactions were incubated at room temperature for 15 min, and 12.5 μL UDP Detection Reagent was added to each reaction. After incubation at room temperature for 1 hr, luminescence was measured on a Plate Reader (SpectraMax i3x, Molecular Devices).

### Cell Culture and Transfection

Cells were cultured in DMEM with 1 g/L glucose, pyruvate and GlutaMAX (Gibco, 10567022), supplemented with 10% heat-inactivated Fetal bovine serum (Gibco 16140071), 200 U/mL penicillin and 200 μg/mL streptomycin (Gibco 15140163). Culture medium was changed daily. Cells were cultured in a 37° C. incubator (Heracell VIOS 160i) with 5% CO₂ and 95% humidity. For subculturing, cells were rinsed with PBS, monodispersed with TrypLE (Gicbo 12604013) and counted on a cell counter (Nexcelom Auto 1000). Cells were discarded when their passage numbers reached 15.

For plasmid transfection in 10 cm dishes, 2×10⁶ HEK293T cells were seeded per dish, and transfection was done after 24 hr (at 40%-60% confluency). Desired amounts of plasmids were dissolved in 100 μL Opti-MEM medium (Gibco 51985034), and TranslT-X2 Transfection Reagent (Mirus Bio, MIR 6000) was added at a ratio of 4 μL of Reagent per 1 μg of DNA. The mixture was gently mixed, incubated at room temperature for 20 min and distributed evenly into the culture dish. Medium was changed to fresh DMEM at 6 hr post-transfection. Plasmid usage in transfection: To target OGT to GFP-β-catenin, 0.1 μg of GFP-β-catenin and 1 μg of each aptamer-encoding plasmid were co-transfected per 10 cm dish. To target OGT to other GFP-tagged proteins, 0.5 μg of GFP-Src, or 1 μg of GFP-ERα/GFP-AMPKα2 and 2 μg of each aptamer-encoding plasmid were co-transfected per 10 cm dish. To target OGT to endogenous β-catenin, 1.5 μg of each plasmid was transfected per 10 cm dish. For cells cultured in different containers, the number of cells, amounts of DNA and transfection reagent were adjusted according to the bottom area of the container.

For cell treatments, Ac₄-5SGlcNAc (Ac5S) was added into medium at 50 μM, for 20 hr; Thiamet-G (TMG) was used at 2 μM for 6 hr; TO1-biotin (TO1) was used at 750 nM for 8 hr. Wnt stimulation was by 50% (v/v) Wnt3A-conditioned medium for 4 hr.

### Generation of Stable Cell Lines

For cells stably expressing T1, NL1F50 and 3JB8F+12: the desired plasmids were transfected into HEK293T cells as above and selected with 500 μg/mL hygromycin B (Gibco 10687010) in culture medium. The selection started at 24 hr post-transfection and lasted for ˜3 weeks, till most of cells died and the survived cells grew confluent. Then the cells were sub-cultured and stored in liquid nitrogen.

Cells with knockdown of β-catenin and shEZH2 were generated by lentiviral transduction.

### Production of Lentivirus

6×10⁵ HEK293T cells were seeded on a 6 cm dish. After 24 hr, 1 μg of shp-catenin (Sigma TRCN0000314921) or 1 μg of shEZH2 (Sigma TRCN0000040077) plasmids was co-transfected with 0.75 μg of psPAX2 (Addgene 12260) and 0.25 μg of pMD2.G (Addgene 12259). Culture medium was replenished at 6 hr post-transfection. Culture continued without medium change, and the first batch of medium was harvested at 48 hr post-transfection. Medium was replenished and the second bath of medium was harvested at 72 hr post-transfection. The two batches were combined, spun at 1250 rpm for 5 min to remove cell pellet, and stored at −80° C. in 1 mL aliquots.

### Lentiviral Transduction and Antibiotic Selection

6×10⁵ HEK293T cells were seeded on a 6 cm dish. After 24 hr, culture medium was changed to fresh DMEM with 8 μg/mL DEAE-Dextran, and 1 mL of thawed lentivirus solutions was added to the cell, and mixed by gently rocking. Culture medium was changed at 24 hr post-transfection. Antibiotic selection started at 48 hr post-transfection with 1 μg/mL puromycin (Sigma-Aldrich P8833) in medium, and continued till most cells died and the survived cells grew confluent. Then the cells were sub-cultured and stored in liquid nitrogen.

### Production of Wnt3A-Conditioned Medium

L Wnt-3A cells (Willert, K., et al. Nature 2003 423:448-452) was sub-cultured at 1:10 in 15 cm dishes. Medium glucose level was monitored twice daily with OneTouch Ultra 2 meter and OneTouch Ultra Blue test strips. When glucose was below 20 mg/dL, it was replenished with 100 g/L glucose solution (Sigma-Aldrich G8270) that was filtered through 0.2 μm PES filter (Whatman 6896-2502), to about 120 mg/dL. The first batch of medium was harvested on the fourth day of culture, and fresh DMEM was added to the cells. Culture continued for another three days, and the second batch of medium was harvested. The two batches were combined, filtered through a 0.2 μm PES bottle-top filter (Fisher Scientific FB12566510), and stored at 4° C. for up to 6 months. This conditioned medium was diluted to 50% (v/v) with regular DMEM for cell treatment.

### Molecular Cloning

For molecular cloning, we used the seamless cloning method using the In-Fusion HD Cloning Plus kit (TaKaRa Bio 638909), following manufacturer's protocol.

Sequences encoding individual and DS aptamers were cloned into the pSilencer2.1-U6 hygro plasmid. The insert fragments were generated in three ways: Klenow extension, Gene Synthesis and PCR amplification. Klenow extension was used for fragments smaller than 100 bp without repetitive sequences. Two DNA oligos representing the 5′-end and 3′-end of the insert with 15-20 bp overhang were synthesized by Integrated DNA Technologies (Coralville, IA, USA). 1 nmol of each oligo was diluted in 20 μL Klenow Annealing Buffer (10 mM Tris-HCl, 10 mM MgCl₂, pH 8.0), denatured at 95° C. for 2 min and annealed to each other at room temperature for 5 min. Extension reactions were carried out using Klenow Fragment (NEB M0210). DNA was purified on 4% agarose gel and extracted with a gel extraction kit (Takara Bio 740609). Inserts with repetitive sequences or longer than 100 bp, and codon-optimized sequences were synthesized by GeneArt (ThermoFisher) and GeneScript (New Jersy, USA). All other inserts were amplified by PCR. 15 bp overhangs with desired linearized vectors were included on both ends of all inserts. Vectors were linearized by inverse PCR using Q5 High-Fidelity DNA Polymerase (NEB M0491). Ligation reactions were conducted with the In-Fusion HD Cloning Plus kit (TaKaRa Bio 638909). After ligation, chemically competent cells (TaKaRa 636766) were transformed with the ligation reaction, and single colonies were picked for sanger-sequencing. Colonies with correct sequences were stored as glycerol stock (20% glycerol) at −80° C. For transfection of cultured mammalian cells, plasmids were purified with the PureLink Low-Endotoxin Midiprep Kit (Invitrogen A35892).

Point mutations were introduced by inverse PCR with overlapping primers containing the desired mutations. Products of inverse PCR were purified on agarose gels, and self-ligated using the In-Fusion HD Cloning Plus kit (TaKaRa Bio 638909). The ligation reactions were transformed into competent cells, and single colonies were picked and sequenced as above.

### Immunoprecipitation (IP), Co-IP and RNA-IP

For immunoprecipitation (IP) of β-catenin, cells were cultured in 10 cm dishes, and harvested with 500 μL RIPA buffer (20 mM Tris-HCl pH 7.5, 150 mM NaCl, 1% (w/v) sodium deoxycholate, 0.1% (w/v) SDS, 1% (v/v) NP-40, 50 mM NaF, 5 mM Na₄P₂O₇, 1 mM EDTA and 1 mM EGTA). 1 mM PMSF, 1 mM DTT, 1 μM PUGNAc, 2 μM TMG, 50 mM GlcNAc, 1× Protease Inhibitor Cocktail (Roche 11873580001) and 1× Phosphatase Inhibitor Cocktail (Thermo Scientific 78426) were freshly added. Cell lysate was sonicated at power 4 for 10 sec (Fisher Scientific F550), centrifuged at 17,000 rcf for 15 min. Supernatant was collected and protein concentration was measured by BCA assay (Thermo Scientific 23227). IP reactions were assembled with 0.5 mg of cell lysate and 0.5 μg of anti-β-catenin antibody (BD Biosciences 610153) or IgG control (Invitrogen MA1-10406), and the volume was adjusted to 0.5 mL with IP buffer (20 mM Tris-HCl pH 7.4, 150 mM NaCl, 1% (v/v) TritonX-100, 50 mM NaF, 5 mM Na₄P₂O₇, 1 mM EDTA and 1 mM EGTA) with inhibitors freshly added. IP reactions were rotated at 4° C. for overnight. On the next day, 7.5 μL Protein A/G magnetic bead slurry (Thermo Scientific 88802) was added, and reactions were rotated at room temperature for 1 hr. Beads were collected on a magnetic stand and washed 5 times with 1 mL IP buffer. Proteins were eluted with 10 μL 2× Laemmli Buffer (Bio-Rad 161-0737) and denatured at 95° C. for 3 min. Denatured samples were loaded on a 4-12% SDS-PAGE gel (Invitrogen WG1402BOX) and proceeded to Western Blot protocols.

For IP and co-immunoprecipitation (Co-IP) of GFP, the same method for IP was used, except cells were lysed with Co-IP Lysis Buffer (150 mM NaCl, 30 mM Tris pH7.5, 1 mM EDTA, 1% TritonX-100, 10% glycerol) with the same inhibitors. IP reactions were assembled with 1 mg of cell lysate and 5 μL of GFP-trap magnetic agarose beads (ProteinTech gtma-20, for GFP-β-catenin), or 2 μg of anti-GFP antibody (Invitrogen G10362, for other GFP-tagged proteins) or IgG control (CST 2729). On the next day, 20 μL Protein A/G magnetic bead slurry (Thermo Scientific 88802) was added to capture the antibody-antigen complex.

For RNA-IP, we adopted the IP protocol with some modifications. 1 mM MgCl₂ was added into the RIPA and IP buffers for “+Mg²⁺” samples. Cells were harvested in RIPA buffer with inhibitors, 100 U/mL RNaseOUT (Invitrogen 10777019) and 2 mM Ribonucleoside Vanadyl Complex (NEB S1402S) freshly added. IP reactions were assembled with 1 mg of cell lysate and 1 μg of anti-OGT antibody (AL24, purified in the lab), and the volumes were adjusted to 1 mL with IP buffer (inhibitors freshly added). For IgG control, 1 μg normal rabbit IgG (Millipore 12-370) was used instead of the antibody. On the next day, 15 μL Protein A/G magnetic bead slurry (Thermo Scientific 88802) was added to capture the antibody-OGT-RNA complex. After washing, residual DNA was digested by 20 units of Turbo DNase (Invitrogen AM2238) in 200 μL Turbo DNase Buffer at 37° C. for 30 min. Then proteins were digested by 100 μg Proteinase K (Invitrogen 25530049) at 50° C. for 30 min. RNA was extracted with Acid-Phenol: Chloroform, pH4.5 (Invitrogen AM9720), precipitated with isopropanol, washed with 70% ethanol, and dissolved in nuclease-free water.

### CpOGA Treatment

CpOGA treatment was performed on total cell lysate before IP. Cells were harvested with Co-IP Lysis Buffer with 1 mM DTT, 1 mM PMSF and 1× Protease Inhibitor Cocktail (Roche 11873580001). PUGNAc and GlcNAc were not included in the lysis buffer so that CpOGA would not be inhibited. 50 μg of purified CpOGA or equal volume of PBS was added to 0.5 mg of lysate, and incubated at 37° C. for 1 hr. Then the reaction was cooled on ice and subjected to IP.

### RT-qPCR

Purified RNA was first converted into cDNA using the SuperScript IV Reverse Transcriptase (Invitrogen 18090010) and RT primers (Table 5). A “RT−” control reaction without the enzyme was included for all samples. Synthesized cDNA was quantified by qPCR on a Stratagene Mx3000P (Agilent Technologies) using TaqMan Fast Advanced Master Mix (Applied Biosystems 4444557), and desired TaqMan assays (Table S6). Amount of residual genomic/plasmid DNA was subtracted from each sample using the readout from its “RT−” control.

### Western Blot

Eluted IP reactions or 20 μg of total cell lysates (in Laemmli Buffer and denatured at 95° C.) were loaded on a 4-12% SDS-PAGE gel (Invitrogen WG1402BOX) and run at 160 V for 90 min. Gel was transferred to a 0.2 μm pore-sized PVDF membrane (GE Healthcare/Cytiva 10600021). Wet transfer was performed in a Criterion Blotter (Bio-Rad 1704070) at 0.45 A for 120 min in Bis-Tris Transfer Buffer (25 mM Bicine, 25 mM Bis-Tris, 1 mM EDTA and 10% (v/v) Methanol). After transfer, membrane was blocked at room temperature for 30 min with rocking, in Blocking Buffer (5% (w/v) BSA (Rockland BSA-1000) dissolved in TBS-T). Blocked membrane was incubated with primary antibody (diluted in Blocking Buffer), at 4° C. for overnight with rocking. On the next day, the membrane was washed for 3 times of 5 min in TBS-T with rocking. Washed membrane was incubated with HRP-conjugated secondary antibody diluted in Blocking Buffer at room temperature for 1 hr with rocking. Then the membrane was washed for 3 times of 5 min, and imaged with ECL reagent (Thermo Scientific 34095) on an iBright FL1500 Imaging system (Invitrogen A44115) or an Amersham Imager 600 RGB (GE Healthcare/Cytiva 29-0834-67) under Chemiluminescence mode.

The images were analyzed using Fiji (Schindelin, J., et al. Nat Methods 2012 9:676-682). Background signal was subtracted using a rolling-ball method (radius=20 or 30), then the lanes were plotted, and band intensities were quantified. To quantify protein-specific O-GlcNAcylation, the band intensities from a O-GlcNAc blot were normalized to those of their corresponding protein-specific blot. Statistical analysis was performed with the GraphPad Prism software.

Dilutions of primary antibodies: anti-O-GlcNAc (CTD110.6)—1:2000; anti-β-catenin (CST 9562)—0.2 μg/mL; anti-Non-phospho (Active) β-Catenin (Ser33/37/Thr41)—1:1000; anti-Non-phospho (Active) β-Catenin (Ser45)—1:1000; anti-phospho-β-Catenin (Ser33/37)—2 μg/mL; anti-phospho-β-Catenin (Ser45)—1:1000; anti-phospho-β-Catenin (Ser552)—1:1000; anti-phospho-β-Catenin (Ser675)—1:1000; anti-OGT (AL28)—1:1000 in 5% (w/v) non-fat milk (Nestle NES22928) in TBS-T; anti-OGA (345)—1:1000 in 5% (w/v) non-fat milk (Nestle NES22928) in TBS-T; anti-EZH2—1:1000; anti-GFP (3E6)—2 μg/mL; anti-β-tubulin—1:1000.

### Mass-Shift Assay

Mass-shift assay was performed according to (Hardiville, S., et al. Mol Cell 2020 77:1143-1152) with the following modifications. Cells were harvested in RIPA buffer, sonicated and cleared as mentioned above. In each GalT labeling reaction, 50 μg of total cell lysate was labeled using the GalT(Y289L) enzyme (a kind gift from Dr. Kelly Moremen at CCRC) and UDP-GalNAz (Chemily SN02012). All 50 μg of the labeled sample was subjected to the SPAAC Click-chemistry reaction, where it was incubated with 10 mM of DBCO-PEG (8.5 kDa) or equal volume of DMSO at 37° C. for 16 hr. PEG-labeled proteins were precipitated with chloroform-methanol and subjected to Western blot. The shifted and unshifted bands of β-catenin was detected with a different antibody (ProteinTech 17565-1-AP). DBCO-PEG (8.5 kDa) was synthesized according to (Hardiville, S., et al. Mol Cell 2020 77:1143-1152), except that Amino-dPEG₁₂-Tris (dPEG₁₂-Tris(m-dPEG₁₁)₃)₃ (Quanta BioDesign 10482) was used.

### Pulse-Chase Experiments on RNA Half-Life

HEK293T stable cell lines that express desired aptamers were cultured in 6-well plate with 1.5 mL medium. Each treatment was replicated in three wells. On day 1, 2.5×10⁵ cells were seeded in each well. Medium was changed daily. On day 3, actinomycin D was added into each well at 5 μg/mL, on a series of timepoints (see Figure legends). When the treatment periods are over, cells were harvested by discarding the medium and adding 750 μL of TRIzol reagent (Invitrogen 15596026) into each well. The lysate was mixed and incubated at room temperature for 5 min and transferred into a Phasemaker tube (Invitrogen A33248). The aqueous phase was separated by adding 150 μL chloroform, thoroughly vertexing and incubating at room temperature for 5 min, and spinning at 12,000 rcf for 15 min at 4° C. The upper aqueous layer was carefully collected without toughing the other layers. RNA was precipitated with isopropanol, washed with 70% ethanol, dissolved in nuclease-free water, and proceeded to RT-qPCR.

### Nucleus/Cytoplasm Fractionation

Nucleus/Cytoplasm Fractionation was performed using the PARIS Kit (Invitrogen AM1921), following the kit manual. 100 U/mL RNaseOUT (Invitrogen 10777019) and 2 mM Ribonucleoside Vanadyl Complex (NEB S1402S) were freshly added to buffers. 2.5×10⁵ cells of the HEK293T stable cell line were cultured for 2 days on 6-well plates, 8 wells in total. Cells were monodispersed with TrypLE (Gibco 12604013) and rinsed with PBS. All steps between cell lysis and addition of 2× Lysis/Binding Solution were performed at 4° C. 1.0×10⁶ cells were collected from each well. For the 4 replicates of Nucleus/Cytoplasm fractionation, cells were lysed by resuspending in 300 μL ice cold Cell Fractionation Buffer and incubating on ice for 10 min. The nuclear and cytoplasmic fractions were separated by spinning at 500 rcf for 5 min. The upper cytoplasmic fraction was carefully collected and transferred into a RNase free tube with equal volume of 2× Lysis/Binding Solution. The pelleted nuclear fraction was washed by resuspending in 300 μL cold Cell Fractionation Buffer and spinning at 500 rcf for 1 min. The nuclear pellet was collected and lysed with 300 μL ice cold Cell Disruption Buffer, followed by 300 μL of 2× Lysis/Binding Solution. For the 4 replicates of total cell lysate (control), collected cells were lysed in 300 μL ice cold Cell Disruption Buffer+300 μL of 2× Lysis/Binding Solution. RNA was purified with the spin columns included in the kit and subjected to RT-qPCR.

### TOPFlash/FOPFlash Luciferase Reporter Assay

Amount and time of transfection for each plasmid is listed in Table S5. On day 1, 2.5×10¹ cells were seeded in each well on 6-well plates. After 24 hr, plasmids encoding the Firefly Luciferase (TOPFlash or FOPFlash) (Veeman, M. T., et al. Current Biology 2003 13:680-685) and the NanoLuc Luciferase (pNL1.1.TK[NIuc/TK], Promega N1501) were transfected into cells. For OGT knockdown and rescue assays, plasmids encoding shOGT and codon-optimized ncOGT were also transfected at this time. On day 3, cells were detached with TrypLE, and 1.5×10⁴ of desired cells were seeded on a 96-well plate in 80 μL medium. On Day 4, cells were transfected with plasmids encoding desired aptamers. On Day 5, culture medium was changed to fresh DMEM (in −Wnt wells) or 50% Wnt3A-conditioned medium (in +Wnt wells) at 20 hr post-transfection. At 24 hr post-transfection, Dual Luciferase Reporter Assay was performed using the Nano-Glo Dual-Luciferase Reporter Assay System (Promega N1521), following the kit manual. Luminescence generated by Firefly Luciferase (TOPFlash or FOPFlash) and NanoLuc Luciferase was measured on a SpectraMax i3× plate reader (Molecular Devices). In data analysis, signal intensity of the Firefly Luciferase was normalized to that of NanoLuc Luciferase in the same well, as a control for variations in transfection efficiency. Then the normalized readings of the TOPFlash wells were normalized again to that of the FOPFlash wells with the same treatments. The second normalization was purposed to control the background (leaky) expression of Firefly Luciferase. Statistical analysis was performed with the GraphPad Prism software.

### Proximity Ligation Assay (PLA)

For PLA, cells were cultured on 8-well Chamber Slides (Thermo Scientific 154941). 1.0×10⁴ cells were seeded in each well with 250 μL medium. After 24 hr, 19.7 ng/well of aptamer-encoding plasmids were transfected into cells (equivalent to 1.5 μg in 10 cm dish). Medium was changed 6 hr after transfection. At 24 hr post-transfection, the plastic chambers were removed, and cells were fixed with 4% formaldehyde (Thermo Scientific 28908, diluted in PBS) at room temperature for 15 min, rinsed twice with PBS, and permeabilized in freshly made Permeabilization Buffer (0.5% Triton X-100 in PBS) at room temperature for 20 min. Cells were washed twice in PBS, barriers were created around each well with a Hydrophobic Barrier Pen (Vector Laboratories H-4000), and proceeded to PLA. PLA was performed using the Duolink Probes, Buffers and Detection Reagents (DU092001, DU092005, DU092013 and DU082049), following the kit manual. Incubation steps were performed in a HybEZ Hybridization Oven with a humidity control tray (ACD 321711). After the final washes, cells were stained with AlexaFluor488-conjugated phalloidin (Invitrogen R37110) or AlexaFluor555-conjugated phalloidin (Invitrogen R37112) for 30 min at room temperature and washed in water for 1 min. Slides were air dried for 15 min at 60° C. in dark. Dried slides were mounted with Prolong Glass Mountant with NucBlue (Hoechst 33342) (Invitrogen P36985) and cover glasses. Mounted slides were left at room temperature for 24 hr before imaging.

Primary antibody usage: anti-β-catenin (BD Biosciences 610153)—1:250; anti-GFP (3E6)—0.8 μg/mL; anti-OGT (AL28)—1:1000; anti-β-TrCP—1:200; anti-EZH2—1:200; anti-EP300—0.5 μg/mL; anti-KAT2A/GCN5L2—1:100; anti-KAT5—1:100; anti-H3K27me3—1:800; anti-H3K9ac—1:400.

Mounted slides were imaged on an Olympus FV1200 confocal microscope using an Olympus UPLFLN40XO lens (40×, NA=1.3, Oil Immersion). Image acquisition was controlled by the Olympus FluoView (Ver. 4.2a) software. Samples were imaged under 405 nm (for NucBlue/Hoechst, HV=650, Gain=1, Offset=5, Laser Power=9.0%), 488 nm (for Phalloidin, HV=630, Gain=1, Offset=5, Laser Power=7.0%) or 559 nm (for Phalloidin, HV=630, Gain=1, Offset=5, Laser Power=5.0%) and 635 nm (for PLA, HV=550, Gain=1, Offset=5, Laser Power=6.0%). The pinhole was set to 1 Airy Unit (80 μm). 12-bit images of the three channels were acquired sequentially.

Analysis of the imaging data was done with Fiji (Schindelin, J., et al. Nat Methods 2012 9:676-682). Total PLA puncta in an image were labeled and counted with the “Find maxima” function (Prominence >500). For nuclear PLA puncta, total puncta in the PLA channel were first labeled in the same way, then the nuclear areas on the Hoechst channel were labeled by adjusting threshold (>500). Nuclear PLA puncta were counted with the “Speckle Inspector” function in the BioVoxxel Toolbox (Brocher, J. EuBIAS-Conference 2015), using the threshold-adjusted Hoechst channel as the “Primary objects”, and the maxima-labeled PLA channel as the “Secondary objects”. Number of nuclei was manually counted. the ratio of total PLA puncta/nuclei, or nuclear PLA/nuclei was calculated for each image. Statistical analysis was performed with GraphPad Prism.

For easy visualization in the Figure panels, image contrast was enhanced by resetting the upper limit of display range in Fiji as follow: Hoechst and Phalloidin channels=2047, PLA channel=1023 (OGT, EZH2, KAT2A and KAT5 Figures) or 2047 (β-TrCP and EP300 Figures). Adjustments were applied only to the presented Figures, not to the original images nor during image analysis.

### RNA Sequencing and Data Analysis

HEK293T cells (wildtype or knockdown of EZH2) were cultured in 10 cm dishes and transfected with desired plasmids. Ac5S and Wnt treatments were performed as described in the Cell Culture and Transfection section. At 24 hr post-transfection, medium was discarded, and cells were lysed using 1 mL of TRIzol Reagent (Invitrogen 15596026) per culture dish. Lysates were mixed by pipetting, transferred into a Phasemaker tube (Invitrogen A33248) and incubated at room temperature for 5 min. The aqueous phase was separated by adding 200 μL chloroform, thoroughly vertexing and spinning at 12,000 rcf for 15 min at 4° C. The upper aqueous layer was carefully collected without touching the other layers. RNA was precipitated with isopropanol, washed with 70% ethanol, dissolved in nuclease-free water, and submitted to GENEWIZ (South Plainfield, NJ, USA) for sequencing. Sequencing was done on an Illumina HiSeq 4000 sequencer in 2×150 bp mode.

Analysis of the RNA-Seq data followed the protocols by Doyle, M., et al. RNA-Seq reads to counts. Galaxy Training Materials 2021. Sequencing adaptors were trimmed from the raw reads using Trim Galore (Martin, M. EMBnet.journal 2011 17; Krueger, F., et al. (2021). Trim Galore!) with parameters (- -paired - -clip_R1 10 - -clip_R2 10 - -three_prime_clip_R1 1 - -three_prime_clip_R2 1). Trimmed reads were mapped to the human reference genome (GRCh38.p13) using HISAT2 (Kim, D., et al. Nat Biotechnol 2019 37:907-915) with parameters (- -dta). Mapped reads were counted to each genes by featureCounts (Liao, Y., et al. Bioinformatics 2014 30:923-930) with parameters (-C -F GTF -g gene_id -p - -countReadPairs -t transcript). The count matrix was annotated using annotateMylDs (Dunning, M. (2017). annotateMylDs). Differential expression of genes was analyzed with DESeq2 (Love, M. I., et al. Genome Biol 2014 15:550) and plotted using EnhancedVolcano (Blighe, K., et al. R package version 1.10.0).

### CUT&RUN Sequencing and Data Analysis

Cleavage Under Targets and Release Using Nuclease (CUT&RUN) was performed using the CUTANA ChIC/CUT&RUN kit (EpiCypher 14-1048), following the kit manual. DTT (1 mM), PUGNAc (1 μM), TMG (2 μM) and Protease Inhibitors Cocktail (1×) were freshly added into all buffers. Cell culture and transfection were carried out on 6-well culture plates. At 24 hr post-transfection, cells were monodispersed by scrapping and pipetting. 5.0×10⁵ cells were used for each reaction. Nuclei of cells were isolated by resuspending 5.0×10⁵ cells in 100 μL of cold Nuclear Extraction Buffer (20 mM HEPES pH 7.9, 10 mM KCl, 0.10% (v/v) Triton X-100, 20% (v/v) glycerol, inhibitors and 0.5 mM Spermidine freshly added) and incubating on ice for 10 min. Nuclei were collected by centrifugation at 600 rcf for 3 min at 4° C., then re-suspended in 100 μL of cold Nuclear Extraction Buffer. Following steps were performed as instructed in the kit manual. 500 ng of EZH2 antibody (CST 5246) was used in each 50 μL of binding reaction. A negative control reaction using 500 ng of rabbit IgG (EpiCypher 13-0042k) was included. After chromatin digestion, 0.5 ng of E. coli genomic DNA (kit included) was spiked-in to each reaction with the Stop Buffer. After chromatin release, DNA fragments were purified by Phenol: Chloroform (Invitrogen 15593031) extraction and isopropanol precipitation. Concentrations of DNA were measured using the Qubit 1×dsDNA HS Assay Kit (Invitrogen Q33230).

Preparation of Sequencing Library was performed using the NEBNext Ultra II DNA Library Prep Kit (NEB E7103L), and NEBNext Multiplex Oligos for Illumina (NEB E6440S), following the protocol Liu, N. (2021). Library Prep for CUT&RUN with NEBNext Ultra II DNA Library Prep Kit for Illumina (E7645) V.2. protocols.io. 6 ng DNA from each CUT&RUN reaction was used as the starting material. A negative control (Input) with 6 ng of sonication-sheard genomic DNA from HEK293T cells was included. Prepared DNA library was dissolved in nuclease-free water, and submitted to GENEWIZ (South Plainfield, NJ, USA) for sequencing. Sequencing was performed on an Illumina HiSeq 4000 sequencer in 2×150 bp mode.

Analysis of the sequencing data followed the protocol by (Zheng, Y., et al. CUT&Tag Data Processing and Analysis Tutorial. protocols.io. 2020). In brief, sequencing adaptors were trimmed from the raw reads using Trim Galore (Martin, M. EMBnet.journal 2011 17; Krueger, F., et al. (2021). Trim Galore!) with parameters (- -paired - -clip_R1 10 - -clip_R2 10 - -three_prime_clip_R1 1 - -three_prime_clip_R2 1). Trimmed reads were mapped to the human reference genome (GRCh38.p13) using Bowtie2 (Langmead, B., et al. Nat Methods 2012 9:357-359) with parameters (-I 10 -X 700 - -no-mixed - -no-discordant - -dovetail - -very-sensitive-local). For spike-in calibration, the trimmed reads were additionally mapped to the E. coli reference genome (ASM584v2) with parameters (-I 10 -X 700 - -no-mixed - -no-discordant - -dovetail - -very-sensitive - -end-to-end), and scale factors were calculated according to the protocol (Zheng, Y., et al. CUT&Tag Data Processing and Analysis Tutorial. protocols.io. 2020). Mapped BAM files were filtered by samtools view (Li, H., et al. Bioinformatics 2009 25:2078-2079) with paramters (-q 20 -F 0x4) and converted to BED format using bedtools bamtobed (Quinlan, A. R., et al. Bioinformatics 2010 26:841-842). The BED files were spike-in calibrated using the calculated scale factors, and converted to genomic coverage bedgraph files using the bedtools genomecov function (Quinlan, A. R., et al. Bioinformatics 2010 26:841-842). Peak calling was performed with SEACR (Meers, M. P., et al. Epigenetics Chromatin 2019b 12:42) under the stringent mode with parameters (-n non -m stringent), using the bedgraph file of IgG as control. Differential binding analysis was performed using Diffbind (Stark, R., et al. DiffBind: differential binding analysis of ChIP-Seq peak data. 2011; Ross-Innes, C. S., et al. Nature 2012 481: 389-393), using the mapped BAM reads (to human and E. coli reference genomes) from Bowtie2 and the called peaks from SEACR as input with parameters (dba.blacklist: blacklist=DBA_BLACKLIST_GRCH38, greylist=FALSE; dba.normalize: method=DBA_DESEQ2, normalize=DBA_NORM_RLE, spikein=TRUE; dba.analyze: method=DBA_DESEQ2). The differential binding sites were annotated with ChlPSeeker (Yu, G., et al. Bioinformatics 2015 31:2382-2383). The heatmap was generated using the dba.plotprofile function in Diffbind, and the profileplyr package (Carroll, TR package version 1.8.1 2021). For visualization, normalized bedgraph files were converted to bigwig files using UCSC bedGraphToBigWig (Kent, W. J., et al. Bioinformatics 2010 26:2204-2207). The genomic browser views were generated by inspecting the bigwig files in the Integrative Genomics Viewer (IGV) (Robinson, J. T., et al. Nat Biotechnol 2011 29:24-26).

### Statistical Analysis

Statistical analysis was performed in the GraphPad Prism 9 software. Unpaired two-tailed Student's t-test was applied to comparisons between two groups. For single-variable comparisons between multiple groups, one-way ANOVA test followed by Dunnett's test comparing to the control groups (T1·AP3 or T1·bc339) was applied. For two-variable comparisons, two-way ANOVA test followed by Turkey's test was applied. Error bars in all plots represents Standard Deviation (SD). For experiments performed with cells, N represents the number of biological replicates. For PLA, N represents the number of different views being analyzed. Details of sample number, data representation and statistical comparison method of each plot can be found in the Figures and legends. ns, p≥0.05; *, p<0.05; **, p<0.01; ***, p<0.001; ****, p<0.0001.

### Design of Riboswitch Elements

To generate the inducible aptamer LRS1F50C, two sets of riboswitch mechanisms each including a mango aptamer (Mango-II_{A22U} or Mango-III_{A10U}) (Trachman, R. J., et al. Biochemistry 2018 57:3544-3548; Trachman, R. J., et al. Nat Chem Biol 2019 15:472-479) and a transmitter element were incorporated into the DS aptamer NL1F50 (FIGS. 7A and 7B). A transmitter element was designed to imperfectly pair with both the core sequence of a Mango aptamer, and with part of T1/AP3. This design allows the transmitter to disrupt the folding of either Mango or T1/AP3 by binding to one of them. This alternative binding is switched by addition/removal of the effector molecule (TO1, abm G955), which facilitates the correct folding of Mango aptamers. In addition, the linker region serves as an insulator, so that the two riboswitch mechanisms do not interfere with each other and can be designed individually.

Design of a transmitter element's sequence was guided by the change of Gibbs Free Energy. For spontaneous processes, the change of Gibbs Free Energy must be negative (ΔΔG<0). In the system consists of an RNA riboswitch (including a sensor aptamer, an actuator aptamer, and a transmitter), a ligand (TO1) and a target protein (OGT or a GFP-tagged protein), the ΔΔG of a conformational change is influenced by 1) the folding of the RNA; and 2) the binding of aptamers to the ligand and the target protein.

Taking the Mango-III_{A10U} riboswitch that regulates T1 as an example, its conformational change triggered by addition of TO1 (from the “ON” state shown in FIG. 7A, to the “OFF” state shown in FIG. 7B) should be a spontaneous process:

$$ΔΔ G ( + TO 1 ) = ΔΔ {G}_{RNA folding} ( ON to OFF ) + ΔΔ {G}_{binding} ( TO 1 ) < 0 \qquad Eq . 1$$

Meanwhile, removal of TO1 triggers the reverse conformational change:

$$ΔΔ G ( + TO 1 ) = ΔΔ {G}_{RNA folding} ( OFF to ON ) + ΔΔ {G}_{binding} ( - TO 1 ) < 0 \qquad Eq \text{.2}$$

Note that

$$ΔΔ {G}_{RNA folding} ( OFF to ON ) + ΔΔ {G}_{binding} ( ON to OFF ) \qquad Eq \text{.3}$$

Eq.1 and Eq.2 are the criteria of a good riboswitch design.

In (1), after addition of TO1:

$$\begin{matrix}ΔΔ {G}_{binding} ( + TO 1 ) = Δ {G}_{binding} ( Mango {III}_{A 10 U} to TO 1 ) \\ - Δ {G}_{binding} ( T 1 to OGT )\end{matrix} \qquad Eq \text{.4}$$

In Eq.2, after removal of TO1:

$$ΔΔ {G}_{binding} ( - TO 1 ) = Δ {G}_{binding} ( T 1 to OGT ) \qquad Eq . 5$$

By combining Eq.1 to Eq.5, we get

$$\begin{matrix}Δ {G}_{binding} ( T 1 to OGT ) < ΔΔ {G}_{binding} ( ON to OFF ) \\ < Δ {G}_{binding} ( T 1 to OGT ) \\ - Δ {G}_{binding} ( Mango {III}_{A 10 U} to TO 1 )\end{matrix} \qquad Eq \text{.6}$$

In a good riboswitch design, its ΔΔG_{RNA folding}(ON to OFF) must satisfy Eq.6.

In Eq.6,

$$ΔΔ {G}_{RNA folding} ( ON to OFF ) = Δ {G}_{RNA folding} ( OFF ) - Δ {G}_{RNA folding} ( ON ) \qquad Eq . 7$$

1) the ΔG_{RNA folding} items are sensitive to the transmitter sequences but insensitive to the presence of TO1, and they were calculated using the RNAfold webserver (Lorenz, R., et al. Algorithms Mol Biol 2011 6:26);
2) the ΔG_{binding} items are insensitive to the transmitter sequences, and they were calculated from the equation:

$$Δ {G}_{binding} = - RT ln {K}_{a} = - RT ln {K}_{d}^{- 1} = RT ln {K}_{d} \qquad Eq \text{.8}$$

In Eq.8, R is the gas constant (8.314 J·K⁻¹·mol⁻¹) and T=298.15 K. The dissociation constants (K_{d}) were measured by SPR (T1 to OGT: 60.49 nM, measured at 298.15 K) or reported previously (AP3 to GFP: 5.1 nM, measured at 295.15 K; Mango-II_{A22u}/Mango-III_{A10U} to TO1: 0.9/1.7 nM, temperature unknown) (Shui, B., et al. Nucleic Acids Res 2012 40:e39; Trachman, R. J., et al. Biochemistry 2018 57:3544-3548; Trachman, R. J., et al. Nat Chem Biol 2019 15:472-479) were used in the calculations.

Sequences of the transmitter elements in both riboswitch mechanisms were manually designed to satisfy formulars Eq.6 to Eq.8.

TABLE 1
Nomenclature of Aptamers in This Paper. Related to all Figures

| Name | Type | Target(s) | Linker Type | Linker Details |
|---|---|---|---|---|
| T1 | Individual | OGT | N/A | N/A |
| AP3 | Individual | GFP | N/A | N/A |
| bc339 | Individual | β-catenin | N/A | N/A |
| 17-6 | Non-aptamer RNA | N/A | N/A | N/A |
| NL1F30/35/50 | Dual-specificity | OGT, | Flexible | 30/35/50 nt |
| | | GFP | | |
| 3JB1F/3JB1R, +2/ | Dual-specificity | OGT, | Folded, | PDB 1MFQ, |
| 4/ . . . /12 | | GFP | 3-way | 0/2/4/ . . . /12 bp |
| | | | | addition on Helix P3 |
| 4JC1RR | Dual-specificity | OGT, | Folded, | PDB 1U9S |
| | | GFP | 4-way | |
| NL8F50/70/100 | Dual-specificity | OGT, | Flexible | 50/70/100 nt |
| | | β-catenin | | |
| 3JB8F + 4 | Dual-specificity | OGT, | Folded, | PDB 1MFQ, |
| | | β-catenin | 3-way | 4 bp addition on Helix P3 |
| 3JB8F + 12/ | Dual-specificity | OGT, | Folded, | PDB 1MFQ, |
| 3JB8R + 12 | | β-catenin | 3-way | 12 bp addition on Helix P3 |
| LRS1F50C | Dual-specificity, | OGT, | Flexible | 50 nt + |
| | Inducible | GFP | | Riboswitches |

^{a} Between the “F” and “R” variants using the same folded linker, the locations of T1 and AP3/bc339 were swapped.

TABLE 2
The SELEX Schemes. Related to FIG. 1 and STAR Methods

| | | | | | |
|---|---|---|---|---|---|
| | | | | | Ampli- |
| | | | | | fica- |
| # | CS | Target | Matrix | Elution | tion |

SELEX scheme that generated T1

| | | | | | |
|---|---|---|---|---|---|
| 1 | 6xHis + MB | TPR | MB | Proteinase K | + |
| 2 | — | OGT-FL | NC | Proteinase K | − |
| 3 | — | TPR | MB | Formamide, 95° C. | + |
| 4 | 6xHis + MB | TPR | NC | Proteinase K | − |
| 5 | — | OGT-FL | MB | Formamide, 95° C. | + |
| 6 | — | OGT-FL | NC | Proteinase K | − |
| 7 | — | TPR | MB | Formamide, 95° C. | + |
| 8 | — | TPR | NC | Proteinase K | − |
| 9 | — | TPR | MB | Formamide, 95° C. | + |
| 10 | — | TPR | NC | Proteinase K | − |
| 11 | — | TPR | MB | Formamide, 95° C. | + |

SELEX scheme that generated bc339

| | | | | | |
|---|---|---|---|---|---|
| 1 | — | β-catenin | MB | 300 mM Imidazole | + |
| 2 | 6xHis + MB | β-catenin | MB | 300 mM Imidazole | + |
| 3 | 6xHis + MB | β-catenin | MB | 300 mM Imidazole | + |
| 4 | — | β-catenin | MB | 300 mM Imidazole | − |
| 5 | — | β-catenin | MB | Formamide, 95° C. | + |
| 6 | — | β-catenin | MB | 300 mM Imidazole | − |
| 7 | — | β-catenin | MB | Formamide, 95° C. | + |
| 8 | — | β-catenin | MB | 300 mM Imidazole | + |

^{a} CS: Counter-selection; MB: Magnetic Beads; NC: Nitrocellulose membrane; TPR: tetratricopeptide repeat domain of OGT; OGT-FL: full-length OGT.

TABLE 3
Expressed Genes Identified in

| | | Base | log2Fold | | | | |
|---|---|---|---|---|---|---|---|
| SYMBOL | GENENAME | Mean | Change | IfcSE | stat | pvalue | padj |
| RBFOX1 | RNA binding fox-1 homolog 1 | 72.51686 | −3.08406 | 0.287009 | −10.7455 | 6.22E−27 | 1.44E−22 |
| CDH18 | cadherin 18 | 31.05052 | −2.82438 | 0.369549 | −7.64278 | 2.13E−14 | 1.64E−10 |
| LRRC4C | leucine rich repeat containing | 27.0728 | −3.6305 | 0.474324 | −7.65404 | 1.95E−14 | 1.64E−10 |
| | 4C | | | | | | |
| NRG3 | neuregulin 3 | 23.06309 | −3.88174 | 0.574711 | −6.75425 | 1.44E−11 | 8.31E−08 |
| PTPRT | protein tyrosine phosphatase, | 18.58714 | −3.56378 | 0.560731 | −6.3556 | 2.08E−10 | 9.61E−07 |
| | receptor type T | | | | | | |
| CTNNA2 | catenin alpha 2 | 24.59054 | −2.82545 | 0.44682 | −6.32345 | 2.56E−10 | 9.87E−07 |
| NRXN1 | neurexin 1 | 18.06252 | −2.92109 | 0.491532 | −5.94282 | 2.80E−09 | 9.27E−06 |
| CNTN5 | contactin 5 | 68.07805 | −1.35194 | 0.22926 | −5.89696 | 3.70E−09 | 1.07E−05 |
| IL1RAPL1 | interleukin 1 receptor accessory | 21.16058 | −2.6309 | 0.456112 | −5.76809 | 8.02E−09 | 2.06E−05 |
| | protein like 1 | | | | | | |
| NA | NA | 41.38799 | −1.62441 | 0.287453 | −5.65103 | 1.59E−08 | 3.69E−05 |
| NEGR1 | neuronal growth regulator 1 | 16.09542 | −2.75519 | 0.498898 | −5.52255 | 3.34E−08 | 6.45E−05 |
| CSMD1 | CUB and Sushi multiple | 18.0899 | −2.70354 | 0.489294 | −5.52539 | 3.29E−08 | 6.45E−05 |
| | domains 1 | | | | | | |
| NA | NA | 19.1005 | −2.65702 | 0.487495 | −5.45035 | 5.03E−08 | 8.95E−05 |
| ADGRB3 | adhesion G protein-coupled | 14.99245 | −3.13988 | 0.585659 | −5.36128 | 8.26E−08 | 0.000137 |
| | receptor B3 | | | | | | |
| NA | NA | 19.89097 | −2.68744 | 0.50401 | −5.33211 | 9.71E−08 | 0.00015 |
| DPP10 | dipeptidyl peptidase like 10 | 19.82954 | −2.61388 | 0.494119 | −5.28999 | 1.22E−07 | 0.000167 |
| LUZP2 | leucine zipper protein 2 | 16.43039 | −2.88605 | 0.544569 | −5.2997 | 1.16E−07 | 0.000167 |
| PCDH11X | protocadherin 11 X-linked | 24.10533 | −2.055 | 0.390944 | −5.2565 | 1.47E−07 | 0.000187 |
| PCDH15 | protocadherin related 15 | 77.67771 | −1.03978 | 0.198809 | −5.23002 | 1.69E−07 | 0.000187 |
| CSMD3 | CUB and Sushi multiple | 17.2439 | −2.55766 | 0.488305 | −5.23784 | 1.62E−07 | 0.000187 |
| | domains 3 | | | | | | |
| NELL1 | neural EGFL like 1 | 19.00684 | −2.5536 | 0.487062 | −5.24285 | 1.58E−07 | 0.000187 |
| CCDC26 | CCDC26 long non-coding RNA | 18.1444 | −2.43791 | 0.471049 | −5.17548 | 2.27E−07 | 0.000239 |
| LOC102546299 | uncharacterized | 14.93627 | −2.84 | 0.553804 | −5.12817 | 2.93E−07 | 0.000294 |
| | LOC102546299 | | | | | | |
| GRIK2 | glutamate ionotropic receptor | 19.45515 | −2.2065 | 0.4344 | −5.07942 | 3.79E−07 | 0.000365 |
| | kainate type subunit 2 | | | | | | |
| CNTNAP5 | contactin associated protein like | 17.43028 | −2.36807 | 0.470286 | −5.03537 | 4.77E−07 | 0.000425 |
| | 5 | | | | | | |
| CALN1 | calneuron 1 | 23.86925 | −1.85937 | 0.368888 | −5.04048 | 4.64E−07 | 0.000425 |
| AGBL1 | ATP/GTP binding protein like 1 | 12.75638 | −3.1832 | 0.638187 | −4.98788 | 6.10E−07 | 0.000523 |
| CADM2 | cell adhesion molecule 2 | 20.42753 | −2.05788 | 0.414618 | −4.96333 | 6.93E−07 | 0.000563 |
| IL1RAPL2 | interleukin 1 receptor accessory | 56.8881 | −1.21518 | 0.245007 | −4.9598 | 7.06E−07 | 0.000563 |
| | protein like 2 | | | | | | |
| UBE2M | ubiquitin conjugating enzyme | 4040.204 | 0.211596 | 0.043655 | 4.847009 | 1.25E−06 | 0.000967 |
| | E2 M | | | | | | |
| GALNT17 | polypeptide N- | 14.48756 | −2.68824 | 0.556549 | −4.83019 | 1.36E−06 | 0.001019 |
| | acetylgalactosaminyltransferase | | | | | | |
| | 17 | | | | | | |
| FRMPD4 | FERM and PDZ domain | 13.28905 | −2.9079 | 0.605875 | −4.7995 | 1.59E−06 | 0.001151 |
| | containing 4 | | | | | | |
| RELN | reelin | 12.28723 | −3.10214 | 0.648124 | −4.78634 | 1.70E−06 | 0.001191 |
| LOC730100 | uncharacterized LOC730100 | 16.84968 | −2.28972 | 0.480958 | −4.76074 | 1.93E−06 | 0.001313 |
| NRXN3 | neurexin 3 | 24.15539 | −1.90071 | 0.402818 | −4.71854 | 2.38E−06 | 0.001571 |
| TAF15 | TATA-box binding protein | 5565.813 | 0.198394 | 0.042262 | 4.694355 | 2.67E−06 | 0.00172 |
| | associated factor 15 | | | | | | |
| NA | NA | 16.90615 | −2.07849 | 0.452062 | −4.59779 | 4.27E−06 | 0.002671 |
| RALYL | RALY RNA binding protein like | 13.08485 | −2.67232 | 0.588286 | −4.54255 | 5.56E−06 | 0.003386 |
| LINC02055 | long intergenic non-protein | 17.34218 | −2.06592 | 0.457322 | −4.51743 | 6.26E−06 | 0.003715 |
| | coding RNA 2055 | | | | | | |
| LINC01317 | long intergenic non-protein | 22.67719 | 1.76665 | 0.393691 | −4.48739 | 7.21E−06 | 0.004173 |
| | coding RNA 1317 | | | | | | |
| NA | NA | 15.31178 | −2.11012 | 0.472015 | −4.47046 | 7.81E−06 | 0.004407 |
| FBXL19 | F-box and leucine rich repeat | 1908.141 | 0.187573 | 0.042062 | 4.459438 | 8.22E−06 | 0.004529 |
| | protein 19 | | | | | | |
| ALK | ALK receptor tyrosine kinase | 12.18239 | −2.51865 | 0.566808 | −4.44356 | 8.85E−06 | 0.004763 |
| NA | NA | 11.86969 | −2.51646 | 0.574763 | −4.37825 | 1.20E−05 | 0.006233 |
| NA | NA | 9.037428 | −3.43501 | 0.785059 | −4.37549 | 1.21E−05 | 0.006233 |
| FBL | fibrillarin | 7152.68 | 0.177845 | 0.040795 | 4.359467 | 1.30E−05 | 0.006561 |
| HDGFL2 | HDGF like 2 | 4444.696 | 0.170692 | 0.039222 | 4.351978 | 1.35E−05 | 0.006645 |
| CNTN4 | contactin 4 | 10.78924 | −2.63041 | 0.606527 | −4.33684 | 1.45E−05 | 0.006872 |
| CTNNA3 | catenin alpha 3 | 123.8136 | −0.68754 | 0.158585 | −4.33546 | 1.45E−05 | 0.006872 |
| GRM5 | glutamate metabotropic | 32.25775 | −1.29993 | 0.301433 | −4.3125 | 1.61E−05 | 0.007473 |
| | receptor 5 | | | | | | |
| CTNND2 | catenin delta 2 | 37.62273 | −1.22704 | 0.285151 | −4.30311 | 1.68E−05 | 0.007645 |
| LOC101927531 | uncharacterized | 4.595515 | −5.67347 | 1.333236 | −4.25541 | 2.09E−05 | 0.009289 |
| | LOC101927531 | | | | | | |
| LINC00276 | long intergenic non-protein | 9.967116 | −3.17217 | 0.751042 | −4.2237 | 2.40E−05 | 0.010497 |
| | coding RNA 276 | | | | | | |
| NA | NA | 9.88788 | −2.83841 | 0.673377 | −4.21518 | 2.50E−05 | 0.010699 |
| NLGN4X | neuroligin 4 X-linked | 7.936501 | −3.70619 | 0.882407 | −4.20009 | 2.67E−05 | 0.01123 |
| DCC | DCC netrin 1 receptor | 9.771696 | −3.08505 | 0.737057 | −4.18564 | 2.84E−05 | 0.011755 |
| NA | NA | 11.22549 | −2.77948 | 0.665747 | −4.17497 | 2.98E−05 | 0.012103 |
| ADCY2 | adenylate cyclase 2 | 10.24226 | −3.05408 | 0.73333 | −4.16468 | 3.12E−05 | 0.012247 |
| SNTG1 | syntrophin gamma 1 | 11.22462 | −2.35709 | 0.566006 | −4.16442 | 3.12E−05 | 0.012247 |
| UQCRB | ubiquinol-cytochrome c | 3811.036 | 0.128364 | 0.03093 | 4.150178 | 3.32E−05 | 0.012817 |
| | reductase binding protein | | | | | | |
| CDH9 | cadherin 9 | 9.171882 | −2.81248 | 0.6821 | −4.12327 | 3.74E−05 | 0.014175 |
| SGCZ | sarcoglycan zeta | 9.961791 | −2.72428 | 0.661464 | −4.11857 | 3.81E−05 | 0.014234 |
| CA10 | carbonic anhydrase 10 | 10.97861 | −2.76394 | 0.672689 | −4.10879 | 3.98E−05 | 0.014544 |
| SENP3 | SUMO specific peptidase 3 | 646.3044 | 0.278562 | 0.067838 | 4.106277 | 4.02E−05 | 0.014544 |
| TMEM132C | transmembrane protein 132C | 8.315541 | −3.49496 | 0.852167 | −4.10126 | 4.11E−05 | 0.014634 |
| NA | NA | 9.757921 | −2.82824 | 0.694846 | −4.07031 | 4.70E−05 | 0.016468 |
| POLR2D | RNA polymerase II subunit D | 2300.047 | 0.155905 | 0.038392 | 4.060816 | 4.89E−05 | 0.016822 |
| GRID2 | glutamate ionotropic receptor | 32.51967 | −1.2005 | 0.296057 | −4.05497 | 5.01E−05 | 0.016822 |
| | delta type subunit 2 | | | | | | |
| SGCD | sarcoglycan delta | 12.31009 | −2.37087 | 0.584464 | −4.05649 | 4.98E−05 | 0.016822 |
| ZNF385D | zinc finger protein 385D | 9.317753 | −2.73283 | 0.675242 | −4.04719 | 5.18E−05 | 0.017143 |
| PKHD1 | PKHD1, fibrocystin/polyductin | 8.975431 | −2.99261 | 0.740699 | −4.04026 | 5.34E−05 | 0.017408 |
| LSAMP | limbic system associated | 57.50758 | −0.87986 | 0.218174 | −4.03284 | 5.51E−05 | 0.017717 |
| | membrane protein | | | | | | |
| DGKB | diacylglycerol kinase beta | 24.47555 | −1.45001 | 0.360714 | −4.01983 | 5.82E−05 | 0.018468 |
| NA | NA | 32.29635 | −1.3306 | 0.333255 | −3.99275 | 6.53E−05 | 0.020431 |
| CDH12 | cadherin 12 | 51.45198 | −0.98885 | 0.249357 | −3.96561 | 7.32E−05 | 0.022299 |
| NA | NA | 11.68701 | −2.39957 | 0.604654 | −3.9685 | 7.23E−05 | 0.022299 |
| SEMA5A | semaphorin 5A | 11.19159 | −2.76232 | 0.698603 | −3.95406 | 7.68E−05 | 0.022803 |
| BASP1 | brain abundant membrane | 7214.52 | 0.130529 | 0.032988 | 3.956928 | 7.59E−05 | 0.022803 |
| | attached signal protein 1 | | | | | | |
| NA | NA | 9.767164 | −2.55263 | 0.648236 | −3.93781 | 8.22E−05 | 0.024095 |
| GRIN2A | glutamate ionotropic receptor | 14.03888 | −1.90031 | 0.482996 | −3.93442 | 8.34E−05 | 0.024133 |
| | NMDA type subunit 2A | | | | | | |
| SORCS1 | sortilin related VPS10 domain | 13.94613 | −1.8989 | 0.483214 | −3.92972 | 8.50E−05 | 0.024305 |
| | containing receptor 1 | | | | | | |
| LINC01435 | long intergenic non-protein | 14.20573 | −1.97862 | 0.505111 | −3.9172 | 8.96E−05 | 0.025289 |
| | coding RNA 1435 | | | | | | |
| LINC01830 | long intergenic non-protein | 6.690036 | −3.53524 | 0.904745 | −3.90744 | 9.33E−05 | 0.026016 |
| | coding RNA 1830 | | | | | | |
| NCAM2 | neural cell adhesion molecule 2 | 7.736622 | −3.41994 | 0.877157 | −3.89889 | 9.66E−05 | 0.026631 |
| UNC13C | unc-13 homolog C | 6.793665 | −3.79397 | 0.977781 | −3.88018 | 0.000104 | 0.028426 |
| HNRNPD | heterogeneous nuclear | 13226.56 | 0.125624 | 0.032503 | 3.865012 | 0.000111 | 0.029901 |
| | ribonucleoprotein D | | | | | | |
| SORCS3 | sortilin related VPS10 domain | 10.1112 | −2.59782 | 0.672797 | −3.86123 | 0.000113 | 0.030018 |
| | containing receptor 3 | | | | | | |
| PTMS | parathymosin | 10365.25 | 0.123502 | 0.032037 | 3.854966 | 0.000116 | 0.030448 |
| NA | NA | 7.621806 | −3.14054 | 0.816729 | −3.84527 | 0.00012 | 0.031321 |
| FLNB | filamin B | 3394.91 | −0.15139 | 0.0396 | −3.82312 | 0.000132 | 0.031996 |
| OPCML | opioid binding protein/cell | 11.57282 | −2.14363 | 0.560534 | −3.82426 | 0.000131 | 0.031996 |
| | adhesion molecule like | | | | | | |
| LINC02008 | long intergenic non-protein | 9.878053 | −2.37352 | 0.618605 | −3.83689 | 0.000125 | 0.031996 |
| | coding RNA 2008 | | | | | | |
| NA | NA | 12.42297 | −2.04949 | 0.535245 | −3.82907 | 0.000129 | 0.031996 |
| NA | NA | 4.221094 | −5.2715 | 1.37489 | −3.83413 | 0.000126 | 0.031996 |
| NA | NA | 11.88035 | −2.24274 | 0.586887 | −3.82141 | 0.000133 | 0.031996 |
| NA | NA | 8.625581 | −2.73195 | 0.713898 | −3.8268 | 0.00013 | 0.031996 |
| NA | NA | 5.263436 | −5.0225 | 1.318416 | −3.8095 | 0.000139 | 0.033232 |
| FGF14 | fibroblast growth factor 14 | 6.349201 | −3.72345 | 0.982091 | −3.79135 | 0.00015 | 0.035393 |
| DISC1FP1 | DISC1 fusion partner 1 | 24.71007 | −1.35804 | 0.358553 | −3.78756 | 0.000152 | 0.035573 |
| SNRNP70 | small nuclear ribonucleoprotein | 9877.997 | 0.129743 | 0.034884 | 3.719249 | 0.0002 | 0.046255 |
| | U1 subunit 70 | | | | | | |
| HIRIP3 | HIRA interacting protein 3 | 1108.763 | 0.203917 | 0.055068 | 3.702991 | 0.000213 | 0.048836 |
| NA | NA | 23.55215 | −1.37028 | 0.370588 | −3.69759 | 0.000218 | 0.049397 |

TABLE 4
TOPFlash Assay Transfections.

| | Amount to | | Day of | | |
|---|---|---|---|---|---|
| Plasmids | transfect | | Transfection | Format | Purpose |
| TOPFlash | 1 | μg | 2 | 6-well plate | Firefly Luciferase |
| FOPFlash | 1 | μg | 2 | 6-well plate | Firefly Luciferase - Leaky Control |
| pNL1.1.TK[Nluc/TK] | 0.5 | ng | 2 | 6-well plate | NanoLuc Luciferase - Transfection Control |
| TRC2-pLKO.5 | 0.5 | μg | 2 | 6-well plate | scrambled RNA |
| TRC2-pLKO.5-shOGT | 0.5 | μg | 2 | 6-well plate | shRNA targeting OGT |
| pcDNA3.1-empty | 0.15 | μg | 2 | 6-well plate | Control plasmid of OGT resscue |
| pcDNA3.1-OGT_opt | 0.15 | μg | 2 | 6-well plate | Codon optimized OGT for rescue |
| T1•bc339 | 11.3 | ng | 4 | 96-well plate | Control aptamers |
| 3JB8F + 12 | 11.3 | ng | 4 | 96-well plate | Dual-specificity Aptamer |

TABLE 5
DNA Oligos for SELEX and TaqMan assays. Related
to STAR Methods and Key Resources Table

| Oligo Name | Sequence (5′ to 3′) |
|---|---|
| L1-N60-ncs | AAGCTTCCGAGCCTTGG-N60- |
| | GGATCCGTAAGTGATTCTCCC |
| | (SEQ ID NO: 1) |
| L2-N50-ncs | GCAGTCACGCAGTGGT-N50- |
| | GTCATCTCATCGCCGCC |
| | (SEQ ID NO: 2) |
| L1-5′-T7-cs | ATCGAGGCTAATACGACTCAC |
| | TATAGGGAGAATCACTTACGG |
| | ATCC (SEQ ID NO: 3) |
| L2-5′-T7-cs | ATCGAGGCTAATACGACTCAC |
| | TATAGGCGGCGATGAGATGAC |
| | (SEQ ID NO: 4) |
| L1-3′-const-ncs | AAG CTT CCG AGC CTT GG |
| | (SEQ ID NO: 5) |
| L2-3′-const-ncs | GCA GTC ACG CAG TGG T |
| | (SEQ ID NO: 6) |

TABLE 6
Aptamer Sequences.

| Aptamer | |
|---|---|
| Names | Sequences (5' to 3') |
| T1 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUU |
| | (SEQ ID NO: 7) |
| AP3 | AGCUUCUGGACUGCGAUGGGAGCACGAAACGUCGUGGCGCAAUUGGGUGGGGAAAGUCCUUAAAAGAG |
| | GGCCACCACAGAAGCU |
| | (SEQ ID NO: 8) |
| 17-6 (Non- | GGCGGCGAUGAGAUGACAGAGAAGCUGAAUUCGCCUCCGUCGGUGGAGAUGCGUAUUUAACCCCGGCA |
| aptamer | CCACUGCGUGACUGC |
| RNA) | (SEQ ID NO: 9) |
| bc339 | AGCUUCUGGGCGGCGAUGAGAUGACGUGUGCGGUGGUCGAGAGGUACCUUGGGUGAGGGAAGGGAAG |
| | GGAGGUUGACCACUGCGUGACUGCCCAGAAGCU |
| | (SEQ ID NO: 10) |
| NL1F30 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACAAAAAAGCUU |
| | CUGAGCUUCUGGACUGCGAUGGGAGCACGAAACGUCGUGGCGCAAUUGGGUGGGGAAAGUCCUUAAAAG |
| | AGGGCCACCACAGAAGCUCAGAAGCU |
| | (SEQ ID NO: 11) |
| NL1F35 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACCAACAAAAAA |
| | AGCUUCUGAGCUUCUGGACUGCGAUGGGAGCACGAAACGUCGUGGCGCAAUUGGGUGGGGAAAGUCCUU |
| | AAAAGAGGGCCACCACAGAAGCUCAGAAGCU |
| | (SEQ ID NO: 12) |
| NL1F50 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACCAACACACACAC |
| | ACACACACAAAAAAGCUUCUGAGCUUCUGGACUGCGAUGGGAGCACGAAACGUCGUGGCGCAAUUGGGU |
| | GGGGAAAGUCCUUAAAAGAGGGCCACCACAGAAGCUCAGAAGCU |
| | (SEQ ID NO: 13) |
| 3JB1F | CGGCAUCAAUAUGGUGACCUCCCGGGCCCGAGGAGAGAAUCACUUACGGAUCCUCGUUAAGUGGCUCUC |
| | AGUCGCCCGUAACUCCGAUGCCGAUGCAGCGGCGACAACUGCACCCAAGGCUCGGAUCCUCGGGAGCG |
| | GGGGACCACCAGGUUGCCUAAGGGGGGUGAACCGGCCCAGGUCGGAAGCUUCUGGACUGCGAUGGGAG |
| | CACGAAACGUCGUGGCGCAAUUGGGUGGGGAAAGUCCUUAAAAGAGGGCCACCACAGAAGCUAACGGAG |
| | CAGGUCAAAACUCCCGUGCUG |
| | (SEQ ID NO: 14) |
| 3JB1F+2 | CGGCAUCAAUAUGGUGACCUCCCGGGCUCCCGAGGAGAGAAUCACUUACGGAUCCUCGUUAAGUGGCUC |
| | UCAGUCGCCCGUAACUCCGAUGCCGAUGCAGCGGCGACAACUGCACCCAAGGCUCGGAUCCUCGGGAGA |
| | GCGGGGGACCACCAGGUUGCCUAAGGGGGGUGAACCGGCCCAGGUCGGAAGCUUCUGGACUGCGAUGG |
| | GAGCACGAAACGUCGUGGCGCAAUUGGGUGGGGAAAGUCCUUAAAAGAGGGCCACCACAGAAGCUAACG |
| | GAGCAGGUCAAAACUCCCGUGCUG |
| | (SEQ ID NO: 15) |
| 3JB1F+4 | CGGCAUCAAUAUGGUGACCUCCCGGGCUAGCCCGAGGAGAGAAUCACUUACGGAUCCUCGUUAAGUGGC |
| | UCUCAGUCGCCCGUAACUCCGAUGCCGAUGCAGCGGCGACAACUGCACCCAAGGCUCGGAUCCUCGGGC |
| | UAGAGCGGGGGACCACCAGGUUGCCUAAGGGGGGUGAACCGGCCCAGGUCGGAAGCUUCUGGACUGCG |
| | AUGGGAGCACGAAACGUCGUGGCGCAAUUGGGUGGGGAAAGUCCUUAAAAGAGGGCCACCACAGAAGCU |
| | AACGGAGCAGGUCAAAACUCCCGUGCUG |
| | (SEQ ID NO: 16) |
| 3JB1F+6 | CGGCAUCAAUAUGGUGACCUCCCGGGCUAGCUCCCGAGGAGAGAAUCACUUACGGAUCCUCGUUAAGUG |
| | GCUCUCAGUCGCCCGUAACUCCGAUGCCGAUGCAGCGGCGACAACUGCACCCAAGGCUCGGAUCCUCGG |
| | GAGCUAGAGCGGGGGACCACCAGGUUGCCUAAGGGGGGUGAACCGGCCCAGGUCGGAAGCUUCUGGAC |
| | UGCGAUGGGAGCACGAAACGUCGUGGCGCAAUUGGGUGGGGAAAGUCCUUAAAAGAGGGCCACCACAGA |
| | AGCUAACGGAGCAGGUCAAAACUCCCGUGCUG |
| | (SEQ ID NO: 17) |
| 3JB1F+8 | CGGCAUCAAUAUGGUGACCUCCCGGGCUAGCUAGCCCGAGGAGAGAAUCACUUACGGAUCCUCGUUAAG |
| | UGGCUCUCAGUCGCCCGUAACUCCGAUGCCGAUGCAGCGGCGACAACUGCACCCAAGGCUCGGAUCCUC |
| | GGGCUAGCUAGAGCGGGGGACCACCAGGUUGCCUAAGGGGGGUGAACCGGCCCAGGUCGGAAGCUUCU |
| | GGACUGCGAUGGGAGCACGAAACGUCGUGGCGCAAUUGGGUGGGGAAAGUCCUUAAAAGAGGGCCACC |
| | ACAGAAGCUAACGGAGCAGGUCAAAACUCCCGUGCUG |
| | (SEQ ID NO: 18) |
| 3JB1F+10 | CGGCAUCAAUAUGGUGACCUCCCGGGCUAGCUAGCUCCCGAGGAGAGAAUCACUUACGGAUCCUCGUUA |
| | AGUGGCUCUCAGUCGCCCGUAACUCCGAUGCCGAUGCAGCGGCGACAACUGCACCCAAGGCUCGGAUCC |
| | UCGGGAGCUAGCUAGAGCGGGGGACCACCAGGUUGCCUAAGGGGGGUGAACCGGCCCAGGUCGGAAGC |
| | UUCUGGACUGCGAUGGGAGCACGAAACGUCGUGGCGCAAUUGGGUGGGGAAAGUCCUUAAAAGAGGGC |
| | CACCACAGAAGCUAACGGAGCAGGUCAAAACUCCCGUGCUG |
| | (SEQ ID NO: 19) |
| 3JB1F+12 | CGGCAUCAAUAUGGUGACCUCCCGGGCUAGCUAGCUAGCCCGAGGAGAGAAUCACUUACGGAUCCUCGU |
| | UAAGUGGCUCUCAGUCGCCCGUAACUCCGAUGCCGAUGCAGCGGCGACAACUGCACCCAAGGCUCGGAU |
| | CCUCGGGCUAGCUAGCUAGAGCGGGGGACCACCAGGUUGCCUAAGGGGGGUGAACCGGCCCAGGUCGG |
| | AAGCUUCUGGACUGCGAUGGGAGCACGAAACGUCGUGGCGCAAUUGGGUGGGGAAAGUCCUUAAAAGAG |
| | GGCCACCACAGAAGCUAACGGAGCAGGUCAAAACUCCCGUGCUG |
| | (SEQ ID NO: 20) |
| 3JB1R | CGGCAUCAAUAUGGUGACCUCCCGGGAGCUUCUGGACUGCGAUGGGAGCACGAAACGUCGUGGCGCAA |
| | UUGGGUGGGGAAAGUCCUUAAAAGAGGGCCACCACAGAAGCUAGCGGGGGACCACCAGGUUGCCUAAGG |
| | GGGGUGAACCGGCCCAGGUCGGACCCGAGGAGAGAAUCACUUACGGAUCCUCGUUAAGUGGCUCUCAG |
| | UCGCCCGUAACUCCGAUGCCGAUGCAGCGGCGACAACUGCACCCAAGGCUCGGAUCCUCGGGAACGGAG |
| | CAGGUCAAAACUCCCGUGCUG |
| | (SEQ ID NO: 21) |
| 3JB1R+2 | CGGCAUCAAUAUGGUGACCUCCCGGGCUAGCUUCUGGACUGCGAUGGGAGCACGAAACGUCGUGGCGC |
| | AAUUGGGUGGGGAAAGUCCUUAAAAGAGGGCCACCACAGAAGCUAGAGCGGGGGACCACCAGGUUGCCU |
| | AAGGGGGGUGAACCGGCCCAGGUCGGACCCGAGGAGAGAAUCACUUACGGAUCCUCGUUAAGUGGCUC |
| | UCAGUCGCCCGUAACUCCGAUGCCGAUGCAGCGGCGACAACUGCACCCAAGGCUCGGAUCCUCGGGAAC |
| | GGAGCAGGUCAAAACUCCCGUGCUG |
| | (SEQ ID NO: 22) |
| 3JB1R+4 | CGGCAUCAAUAUGGUGACCUCCCGGGCUAGAGCUUCUGGACUGCGAUGGGAGCACGAAACGUCGUGGC |
| | GCAAUUGGGUGGGGAAAGUCCUUAAAAGAGGGCCACCACAGAAGCUCUAGAGCGGGGGACCACCAGGUU |
| | GCCUAAGGGGGGUGAACCGGCCCAGGUCGGACCCGAGGAGAGAAUCACUUACGGAUCCUCGUUAAGUG |
| | GCUCUCAGUCGCCCGUAACUCCGAUGCCGAUGCAGCGGCGACAACUGCACCCAAGGCUCGGAUCCUCGG |
| | GAACGGAGCAGGUCAAAACUCCCGUGCUG |
| | (SEQ ID NO: 23) |
| 3JB1R+6 | CGGCAUCAAUAUGGUGACCUCCCGGGCUAGCUAGCUUCUGGACUGCGAUGGGAGCACGAAACGUCGUG |
| | GCGCAAUUGGGUGGGGAAAGUCCUUAAAAGAGGGCCACCACAGAAGCUAGCUAGAGCGGGGGACCACCA |
| | GGUUGCCUAAGGGGGGUGAACCGGCCCAGGUCGGACCCGAGGAGAGAAUCACUUACGGAUCCUCGUUA |
| | AGUGGCUCUCAGUCGCCCGUAACUCCGAUGCCGAUGCAGCGGCGACAACUGCACCCAAGGCUCGGAUCC |
| | UCGGGAACGGAGCAGGUCAAAACUCCCGUGCUG |
| | (SEQ ID NO: 24) |
| 3JB1R+8 | CGGCAUCAAUAUGGUGACCUCCCGGGCUAGCUAGAGCUUCUGGACUGCGAUGGGAGCACGAAACGUCG |
| | UGGCGCAAUUGGGUGGGGAAAGUCCUUAAAAGAGGGCCACCACAGAAGCUCUAGCUAGAGCGGGGGACC |
| | ACCAGGUUGCCUAAGGGGGGUGAACCGGCCCAGGUCGGACCCGAGGAGAGAAUCACUUACGGAUCCUC |
| | GUUAAGUGGCUCUCAGUCGCCCGUAACUCCGAUGCCGAUGCAGCGGCGACAACUGCACCCAAGGCUCGG |
| | AUCCUCGGGAACGGAGCAGGUCAAAACUCCCGUGCUG |
| | (SEQ ID NO: 25) |
| 3JB1R+10 | CGGCAUCAAUAUGGUGACCUCCCGGGCUAGCUAGCUAGCUUCUGGACUGCGAUGGGAGCACGAAACGUC |
| | GUGGCGCAAUUGGGUGGGGAAAGUCCUUAAAAGAGGGCCACCACAGAAGCUAGCUAGCUAGAGCGGGG |
| | GACCACCAGGUUGCCUAAGGGGGGUGAACCGGCCCAGGUCGGACCCGAGGAGAGAAUCACUUACGGAU |
| | CCUCGUUAAGUGGCUCUCAGUCGCCCGUAACUCCGAUGCCGAUGCAGCGGCGACAACUGCACCCAAGGC |
| | UCGGAUCCUCGGGAACGGAGCAGGUCAAAACUCCCGUGCUG |
| | (SEQ ID NO: 26) |
| 3JB1R+12 | CGGCAUCAAUAUGGUGACCUCCCGGGCUAGCUAGCUAGAGCUUCUGGACUGCGAUGGGAGCACGAAACG |
| | UCGUGGCGCAAUUGGGUGGGGAAAGUCCUUAAAAGAGGGCCACCACAGAAGCUCUAGCUAGCUAGAGCG |
| | GGGGACCACCAGGUUGCCUAAGGGGGGUGAACCGGCCCAGGUCGGACCCGAGGAGAGAAUCACUUACG |
| | GAUCCUCGUUAAGUGGCUCUCAGUCGCCCGUAACUCCGAUGCCGAUGCAGCGGCGACAACUGCACCCAA |
| | GGCUCGGAUCCUCGGGAACGGAGCAGGUCAAAACUCCCGUGCUG |
| | (SEQ ID NO: 27) |
| 4JC1RR | GGAAAGUGCCACAGAGAAGAGACCGCCAGCUUCUGGACUGCGAUGGGAGCACGAAACGUCGUGGCGCAA |
| | UUGGGUGGGGAAAGUCCUUAAAAGAGGGCCACCACAGAAGCUGGCAAGGGUGAAACGGGGGGUAACCC |
| | GAGGAGAGAAUCACUUACGGAUCCUCGUUAAGUGGCUCUCAGUCGCCCGUAACUCCGAUGCCGAUGCAG |
| | CGGCGACAACUGCACCCAAGGCUCGGAUCCUCGGGGAGCCCACCGCCUGGCCUGGCAGCUUCUGGACU |
| | GCGAUGGGAGCACGAAACGUCGUGGCGCAAUUGGGUGGGGAAAGUCCUUAAAAGAGGGCCACCACAGAA |
| | GCUAACAGGCCGGGGCACGGCAAACCCCCCGAGGAGAGAAUCACUUACGGAUCCUCGUUAAGUGGCUCU |
| | CAGUCGCCCGUAACUCCGAUGCCGAUGCAGCGGCGACAACUGCACCCAAGGCUCGGAUCCUCGGG |
| | (SEQ ID NO: 28) |
| NL8F50 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACCAACACACACAC |
| | ACACACACAAAAAAGCUUCUGGGCGGCGAUGAGAUGACGUGUGCGGUGGUCGAGAGGUACCUUGGGUG |
| | AGGGAAGGGAAGGGAGGUUGACCACUGCGUGACUGCCCAGAAGCU |
| | (SEQ ID NO: 29) |
| NL8F70 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACCAACACACACAC |
| | ACACACACACACACACACCAACACACACAAAAAAGCUUCUGGGCGGCGAUGAGAUGACGUGUGCGGUGGU |
| | CGAGAGGUACCUUGGGUGAGGGAAGGGAAGGGAGGUUGACCACUGCGUGACUGCCCAGAAGCU |
| | (SEQ ID NO: 30) |
| NL8F100 | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAAGCUUCAACACACACACACACACACACACACACACCAACACACACAC |
| | ACACACACACACACACACCAACACACACACACACACACACACACACACCAACACACACAAAAAAGCUUCUG |
| | GGCGGCGAUGAGAUGACGUGUGCGGUGGUCGAGAGGUACCUUGGGUGAGGGAAGGGAAGGGAGGUUG |
| | ACCACUGCGUGACUGCCCAGAAGCU |
| | (SEQ ID NO: 31) |
| 3JB8F+4 | CGGCAUCAAUAUGGUGACCUCCCGGGCUAGCCCGAGGAGAGAAUCACUUACGGAUCCUCGUUAAGUGGC |
| | UCUCAGUCGCCCGUAACUCCGAUGCCGAUGCAGCGGCGACAACUGCACCCAAGGCUCGGAUCCUCGGGC |
| | UAGAGCGGGGGACCACCAGGUUGCCUAAGGGGGGUGAACCGGCCCAGGUCGGAGCUUCUGGGCGGCGA |
| | UGAGAUGACGUGUGCGGUGGUCGAGAGGUACCUUGGGUGAGGGAAGGGAAGGGAGGUUGACCACUGCG |
| | UGACUGCCCAGAAGCAACGGAGCAGGUCAAAACUCCCGUGCUG |
| | (SEQ ID NO: 32) |
| 3JB8F+12 | CGGCAUCAAUAUGGUGACCUCCCGGGCUAGCUAGCUAGCCCGAGGAGAGAAUCACUUACGGAUCCUCGU |
| | UAAGUGGCUCUCAGUCGCCCGUAACUCCGAUGCCGAUGCAGCGGCGACAACUGCACCCAAGGCUCGGAU |
| | CCUCGGGCUAGCUAGCUAGAGCGGGGGACCACCAGGUUGCCUAAGGGGGGUGAACCGGCCCAGGUCGG |
| | AGCUUCUGGGCGGCGAUGAGAUGACGUGUGCGGUGGUCGAGAGGUACCUUGGGUGAGGGAAGGGAAG |
| | GGAGGUUGACCACUGCGUGACUGCCCAGAAGCAACGGAGCAGGUCAAAACUCCCGUGCUG |
| | (SEQ ID NO: 33) |
| 3JB8R+12 | CGGCAUCAAUAUGGUGACCUCCCGGGCUAGCUAGCUAGGCUUCUGGGCGGCGAUGAGAUGACGUGUGC |
| | GGUGGUCGAGAGGUACCUUGGGUGAGGGAAGGGAAGGGAGGUUGACCACUGCGUGACUGCCCAGAAGC |
| | CUAGCUAGCUAGAGCGGGGGACCACCAGGUUGCCUAAGGGGGGUGAACCGGCCCAGGUCGGACCCGAG |
| | GAGAGAAUCACUUACGGAUCCUCGUUAAGUGGCUCUCAGUCGCCCGUAACUCCGAUGCCGAUGCAGCGG |
| | CGACAACUGCACCCAAGGCUCGGAUCCUCGGGAACGGAGCAGGUCAAAACUCCCGUGCUG |
| | (SEQ ID NO: 34) |
| LRS1F50C | GGGAGAAUCACUUACGGAUCCUCGUUAAGUGGCUCUCAGUCGCCCGUAACUCCGAUGCCGAUGCAGCGG |
| | CGACAACUGCACCCAAGGCUCGGAGAAGGAAGGUUUGGUAUGUGGUAUAUUCUCCGAGCCCAACACACA |
| | CACACACACACACACACACACAACACACACACACACACACAAGCUUCUGAGCUGACUGCGAUGGGAGCAC |
| | GAAACGUCGUGGCGCAAUUGGGUGGGGAAAGUCCUUAAAAGAGGGCCACCAAGCUCAGAAGCUGAAGGA |
| | GAGGAGAGGUAGAGGAGAAGCUUCUGAGCUUGG |
| | (SEQ ID NO: 35) |
| atdT1 | TTGTCGCCGCAGCATCGGCAACGGAGTTACCGGCGACTGACAGCCACTTATCGAGGAT |
| | (SEQ ID NO: 36) |

### Example 2: Aptamer Binding

Tables 7 to 9 show RNA aptamer binding for novel β-catenin, OGT, and OGA aptamers.

TABLE 7
β-catenin aptamers

| Aptamer | Protein | kₐ (1/Ms) | k_{d} (1/s) | K_{D} (M) |
|---|---|---|---|---|
| bc1 | β-catenin | 1.512E+5 | 2.764E−4 | 1.828E−9 |
| bc2 | β-catenin | 1.546E+5 | 2.919E−4 | 1.889E−9 |

TABLE 8
OGT Aptamers

| Aptamer | Protein | kₐ (1/Ms) | k_{d} (1/s) | K_{D} (M) |
|---|---|---|---|---|
| OGT1 | OGT-FL | 2.353E+5 | 0.008204 | 3.487E−8 |
| OGT2 | OGT-FL | 8.269E+4 | 0.001066 | 1.290E−8 |
| OGT3 | OGT-FL | 1.162E+6 | 0.001720 | 1.480E−9 |
| OGT4 | OGT-FL | 2.158E+4 | 0.001356 | 6.282E−8 |
| OGT7 | OGT-FL | 2.814E+5 | 9.598E−4 | 3.411E−9 |
| OGT8 | OGT-FL | 8.585E+4 | 6.203E−4 | 7.226E−9 |
| OGT9 | OGT-FL | 1.636E+5 | 9.905E−4 | 6.053E−9 |

TABLE 9
OGA Aptamers

| Aptamer | Protein | kₐ (1/Ms) | k_{d} (1/s) | K_{D} (M) |
|---|---|---|---|---|
| OGA1 | OGA-FL | 3.439E+5 | 4.312E−4 | 1.254E−9 |
| OGA2 | OGA-FL | 1.873E+6 | 7.218E−4 | 3.854E−10 |
| OGA3 | OGA-FL | 1.193E+6 | 6.978E−4 | 5.848E−10 |
| OGA4 | OGA-FL | 4.253E+6 | 0.002640 | 6.208E−10 |
| OGA6 | OGA-FL | 1.177E+6 | 5.731E−4 | 4.870E−10 |
| HAT-1 | OGA-FL | 1.085E+6 | 0.001031 | 9.507E−10 |
| HAT-2 | OGA-FL | 3.924E+5 | 2.888E−4 | 7.360E−10 |
| HAT-12 | OGA-FL | 3.404E+5 | 4.097E−4 | 1.204E−9 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

The invention claimed is:

1. A dual-specificity aptamer comprising a first RNA aptamer sequence that targets the nucleocytoplasmic isoform of O-GlcNAc transferase (OGT) or O-GlcNAcase (OGA) and a second RNA aptamer sequence, wherein the first RNA aptamer sequence and the second RNA aptamer sequence are connected by a linker region,
wherein the first RNA aptamer comprises the nucleic acid sequence SEQ ID NO:7, 42, 43, 44, 45, 46, 47, or 48, or a variant and/or fragment thereof at least 90 nucleotides in length having at least 90% sequence identity to SEQ ID NO: 7, 42, 43, 44, 45, 46, 47, or 48 that is able to bind OGT, or
wherein the first RNA aptamer comprises the nucleic acid sequence SEQ ID NO:49, 50, 51, 52, 53, 54, 55, or 56, or a variant and/or fragment thereof at least 90 nucleotides in length having at least 90% sequence identity to SEQ ID NO:49, 50, 51, 52, 53, 54, 55, or 56 that is able to bind OGA.

2-7. (canceled)

8. The dual-specificity aptamer of claim 1, wherein the second RNA aptamer comprises the nucleic acid sequence SEQ ID NO:10, or a variant and/or fragment thereof at least 74 nucleotides in length having at least 90% sequence identity to SEQ ID NO:10 that is able to selectively bind β-catenin.

9. (canceled)

10. The dual-specificity aptamer of claim 1, wherein the second RNA aptamer comprises the nucleic acid sequence SEQ ID NO:8, or a variant and/or fragment thereof at least 74 nucleotides in length having at least 90% sequence identity to SEQ ID NO:8, that is able to selectively bind green fluorescent protein (GFP).

11. The dual-specificity aptamer of claim 1, wherein the second RNA aptamer sequence is generated for a target protein using Systematic Evolution of Ligands by EXponential enrichment.

12. The dual-specificity aptamer of claim 1, wherein the linker region is selected from a flexible linker and a folded linker.

13. The dual-specificity aptamer of claim 12, wherein the flexible linker is from 10 to 100 nucleotides in length.

14. The dual-specificity aptamer of claim 12, wherein the linker comprises three-way RNA junctions or four-way RNA junctions.

15. The dual-specificity aptamer of claim 1, wherein the dual-specificity aptamer comprises a ligand-responsive riboswitch comprising a sensor sequence that activates or deactivates the dual-specificity aptamer upon binding of the ligand to the sensor sequence.

16. The dual-specificity aptamer of claim 15, wherein the ligand-responsive riboswitch is selected from the group consisting of a theophylline riboswitch, a thiamine pyrophosphate (TPP) riboswitch, an adenosine cobalamin (AdoCbl) riboswitch, an S-adenosyl methionine (SAM) riboswitch, an SAH riboswitch, a flavin mononucleotide (FMN) riboswitch, a tetrahydrofolate riboswitch, a lysine riboswitch, a glycine riboswitch, a purine riboswitch, a guanine riboswitch, a GlmS riboswitch, or a pre-queosine1 (PreQ1) riboswitch.

17. A vector comprising a nucleic acid sequence encoding the dual dual-specificity aptamer of claim 1 operably linked to an expression control system.

18. The vector of claim 17, wherein the expression control system comprises an inducible promoter or a tissue specific promoter.

19. A composition comprising the dual-specificity aptamer of claim 1 in a pharmaceutically acceptable carrier.

20. A method of modulating O-GlcNAc on a protein, comprising contacting the protein with the composition of claim 19.

21. An RNA aptamer comprising:
the nucleic acid sequence SEQ ID NO:7, or a variant and/or fragment thereof at least 90 nucleotides in length having at least 90% sequence identity to SEQ ID NO:7 that is able to bind O-GlcNAc transferase (OGT), or
the nucleic acid sequence SEQ ID NO:49, 50, 51, 52, 53, 54, 55, or 56, or a variant and/or fragment thereof at least 90 nucleotides in length having at least 90% sequence identity to SEQ ID NO:49, 50, 51, 52, 53, 54, 55, or 56 that is able to bind O-GlcNAcase (OGA).

22. (canceled)

23. An RNA aptamer comprising the nucleic acid sequence SEQ ID NO:10, 40, or 41, or a variant and/or fragment thereof at least 74 nucleotides in length having at least 90% sequence identity to SEQ ID NO:10, 40, or 41 that is able to selectively bind β-catenin.


SEQUENCE LISTING (69 sequences)

SEQ ID NO: 1 - DNA, 98 bp, synthetic construct
  source 1..98: mol_type = other DNA
aagcttccgagccttggnnnnnnnnnnnnnnnnnnnnnnnnnnnnnnnnnnnnnnnnnnnnnnnnnnnnnnnnnnnnggatccgtaagtgattctccc

SEQ ID NO: 2 - DNA, 83 bp, synthetic construct
  source 1..83: mol_type = other DNA
gcagtcacgcagtggtnnnnnnnnnnnnnnnnnnnnnnnnnnnnnnnnnnnnnnnnnnnnnnnnnngtcatctcatcgccgcc

SEQ ID NO: 3 - DNA, 46 bp, synthetic construct
  source 1..46: mol_type = other DNA
atcgaggctaatacgactcactatagggagaatcacttacggatcc

SEQ ID NO: 4 - DNA, 42 bp, synthetic construct
  source 1..42: mol_type = other DNA
atcgaggctaatacgactcactataggcggcgatgagatgac

SEQ ID NO: 5 - DNA, 17 bp, synthetic construct
  source 1..17: mol_type = other DNA
aagcttccgagccttgg

SEQ ID NO: 6 - DNA, 16 bp, synthetic construct
  source 1..16: mol_type = other DNA
gcagtcacgcagtggt

SEQ ID NO: 7 - RNA, 98 nt, synthetic construct
  source 1..98: mol_type = other RNA
gggagaatcacttacggatcctcgttaagtggctctcagtcgcccgtaactccgatgccgatgcagcggcgacaactgcacccaaggctcggaagctt

SEQ ID NO: 8 - RNA, 84 nt, synthetic construct
  source 1..84: mol_type = other RNA
agcttctggactgcgatgggagcacgaaacgtcgtggcgcaattgggtggggaaagtccttaaaagagggccaccacagaagct

SEQ ID NO: 9 - RNA, 83 nt, synthetic construct
  source 1..83: mol_type = other RNA
ggcggcgatgagatgacagagaagctgaattcgcctccgtcggtggagatgcgtatttaaccccggcaccactgcgtgactgc

SEQ ID NO: 10 - RNA, 100 nt, synthetic construct
  source 1..100: mol_type = other RNA
agcttctgggcggcgatgagatgacgtgtgcggtggtcgagaggtaccttgggtgagggaagggaagggaggttgaccactgcgtgactgcccagaagct

SEQ ID NO: 11 - RNA, 233 nt, synthetic construct
  source 1..233: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacacaaaaaagcttctgagcttctggactgcgatgggagcacgaaacgtcgtggcgcaattgggtggggaaagtccttaaaagagggccaccacagaagctcagaagct

SEQ ID NO: 12 - RNA, 238 nt, synthetic construct
  source 1..238: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacaccaacaaaaaaagcttctgagcttctggactgcgatgggagcacgaaacgtcgtggcgcaattgggtggggaaagtccttaaaagagggccaccacagaagctcagaagct

SEQ ID NO: 13 - RNA, 253 nt, synthetic construct
  source 1..253: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacaccaacacacacacacacacacaaaaaagcttctgagcttctggactgcgatgggagcacgaaacgtcgtggcgcaattgggtggggaaagtccttaaaagagggccaccacagaagctcagaagct

SEQ ID NO: 14 - RNA, 295 nt, synthetic construct
  source 1..295: mol_type = other RNA
cggcatcaatatggtgacctcccgggcccgaggagagaatcacttacggatcctcgttaagtggctctcagtcgcccgtaactccgatgccgatgcagcggcgacaactgcacccaaggctcggatcctcgggagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 15 - RNA, 299 nt, synthetic construct
  source 1..299: mol_type = other RNA
cggcatcaatatggtgacctcccgggctcccgaggagagaatcacttacggatcctcgttaagtggctctcagtcgcccgtaactccgatgccgatgcagcggcgacaactgcacccaaggctcggatcctcgggagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 16 - RNA, 303 nt, synthetic construct
  source 1..303: mol_type = other RNA
cggcatcaatatggtgacctcccgggctagcccgaggagagaatcacttacggatcctcgttaagtggctctcagtcgcccgtaactccgatgccgatgcagcggcgacaactgcacccaaggctcggatcctcgggctagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 17 - RNA, 307 nt, synthetic construct
  source 1..307: mol_type = other RNA
cggcatcaatatggtgacctcccgggctagctcccgaggagagaatcacttacggatcctcgttaagtggctctcagtcgcccgtaactccgatgccgatgcagcggcgacaactgcacccaaggctcggatcctcgggagctagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 18 - RNA, 311 nt, synthetic construct
  source 1..311: mol_type = other RNA
cggcatcaatatggtgacctcccgggctagctagcccgaggagagaatcacttacggatcctcgttaagtggctctcagtcgcccgtaactccgatgccgatgcagcggcgacaactgcacccaaggctcggatcctcgggctagctagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 19 - RNA, 315 nt, synthetic construct
  source 1..315: mol_type = other RNA
cggcatcaatatggtgacctcccgggctagctagctcccgaggagagaatcacttacggatcctcgttaagtggctctcagtcgcccgtaactccgatgccgatgcagcggcgacaactgcacccaaggctcggatcctcgggagctagctagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 20 - RNA, 319 nt, synthetic construct
  source 1..319: mol_type = other RNA
cggcatcaatatggtgacctcccgggctagctagctagcccgaggagagaatcacttacggatcctcgttaagtggctctcagtcgcccgtaactccgatgccgatgcagcggcgacaactgcacccaaggctcggatcctcgggctagctagctagagcgggggaccaccaggttgcctaaggggggtgaaccggcccaggtcggaagcttctggactgcgatgggagcacgaaacgtcgtggcgcaattgggtggggaaagtccttaaaagagggccaccacagaagctaacggagcaggtcaaaactcccgtgctg

SEQ ID NO: 21 - RNA, 295 nt, synthetic construct
  source 1..295: mol_type = other RNA
cggcatcaatatggtgacctcccgggagcttctggactgcgatgggagcacgaaacgtcgtggcgcaattgggtggggaaagtccttaaaagagggccaccacagaagctagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 22 - RNA, 299 nt, synthetic construct
  source 1..299: mol_type = other RNA
cggcatcaatatggtgacctcccgggctagcttctggactgcgatgggagcacgaaacgtcgtggcgcaattgggtggggaaagtccttaaaagagggccaccacagaagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 23 - RNA, 303 nt, synthetic construct
  source 1..303: mol_type = other RNA
cggcatcaatatggtgacctcccgggctagagcttctggactgcgatgggagcacgaaacgtcgtggcgcaattgggtggggaaagtccttaaaagagggccaccacagaagctctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 24 - RNA, 307 nt, synthetic construct
  source 1..307: mol_type = other RNA
cggcatcaatatggtgacctcccgggctagctagcttctggactgcgatgggagcacgaaacgtcgtggcgcaattgggtggggaaagtccttaaaagagggccaccacagaagctagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 25 - RNA, 311 nt, synthetic construct
  source 1..311: mol_type = other RNA
cggcatcaatatggtgacctcccgggctagctagagcttctggactgcgatgggagcacgaaacgtcgtggcgcaattgggtggggaaagtccttaaaagagggccaccacagaagctctagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 26 - RNA, 315 nt, synthetic construct
  source 1..315: mol_type = other RNA
cggcatcaatatggtgacctcccgggctagctagctagcttctggactgcgatgggagcacgaaacgtcgtggcgcaattgggtggggaaagtccttaaaagagggccaccacagaagctagctagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 27 - RNA, 319 nt, synthetic construct
  source 1..319: mol_type = other RNA
cggcatcaatatggtgacctcccgggctagctagctagagcttctggactgcgatgggagcacgaaacgtcgtggcgcaattgggtggggaaagtccttaaaagagggccaccacagaagctctagctagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 28 - RNA, 478 nt, synthetic construct
  source 1..478: mol_type = other RNA
ggaaagtgccacagagaagagaccgccagcttctggactgcgatgggagcacgaaacgtcgtggcgcaattgggtggggaaagtccttaaaagagggccaccacagaagctggcaagggtgaaacggcggggtaacccgaggagagaatcacttacggatcctcgttaagtggctctcagtcgcccgtaactccgatgccgatgcagcggcgacaactgcacccaaggctcggatcctcggggagcccaccgcctggcctggcagcttctggactgcgatgggagcacgaaacgtcgtggcgcaattgggtggggaaagtccttaaaagagggccaccacagaagctaacaggccggggcacggcaaaccccccgaggagagaatcacttacggatcctcgttaagtggctctcagtcgcccgtaactccgatgccgatgcagcggcgacaactgcacccaaggctcggatcctcggg

SEQ ID NO: 29 - RNA, 253 nt, synthetic construct
  source 1..253: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacaccaacacacacacacacacacaaaaaagcttctgggcggcgatgagatgacgtgtgcggtggtcgagaggtaccttgggtgagggaagggaagggaggttgaccactgcgtgactgcccagaagct

SEQ ID NO: 30 - RNA, 273 nt, synthetic construct
  source 1..273: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacaccaacacacacacacacacacacacacacaccaacacacacaaaaaagcttctgggcggcgatgagatgacgtgtgcggtggtcgagaggtaccttgggtgagggaagggaagggaggttgaccactgcgtgactgcccagaagct

SEQ ID NO: 31 - RNA, 303 nt, synthetic construct
  source 1..303: mol_type = other RNA
gggagaatcacttacggatcctcgttaagtggctctcagtcgcccgtaactccgatgccgatgcagcggcgacaactgcacccaaggctcggaagcttcaacacacacacacacacacacacacacaccaacacacacacacacacacacacacacaccaacacacacacacacacacacacacacaccaacacacacaaaaaagcttctgggcggcgatgagatgacgtgtgcggtggtcgagaggtaccttgggtgagggaagggaagggaggttgaccactgcgtgactgcccagaagct

SEQ ID NO: 32 - RNA, 317 nt, synthetic construct
  source 1..317: mol_type = other RNA
cggcatcaatatggtgacctcccgggctagcccgaggagagaatcacttacggatcctcgttaagtggctctcagtcgcccgtaactccgatgccgatgcagcggcgacaactgcacccaaggctcggatcctcgggctagagcgggggaccaccaggttgcctaaggggggtgaaccggcccaggtcggagcttctgggcggcgatgagatgacgtgtgcggtggtcgagaggtaccttgggtgagggaagggaagggaggttgaccactgcgtgactgcccagaagcaacggagcaggtcaaaactcccgtgctg

SEQ ID NO: 33 - RNA, 333 nt, synthetic construct
  source 1..333: mol_type = other RNA
cggcatcaatatggtgacctcccgggctagctagctagcccgaggagagaatcacttacggatcctcgttaagtggctctcagtcgcccgtaactccgatgccgatgcagcggcgacaactgcacccaaggctcggatcctcgggctagctagctagagcgggggaccaccaggttgcctaaggggggtgaaccggcccaggtcggagcttctgggcggcgatgagatgacgtgtgcggtggtcgagaggtaccttgggtgagggaagggaagggaggttgaccactgcgtgactgcccagaagcaacggagcaggtcaaaactcccgtgctg

SEQ ID NO: 34 - RNA, 333 nt, synthetic construct
  source 1..333: mol_type = other RNA
cggcatcaatatggtgacctcccgggctagctagctaggcttctgggcggcgatgagatgacgtgtgcggtggtcgagaggtaccttgggtgagggaagggaagggaggttgaccactgcgtgactgcccagaagcctagctagctagagcgggggaccaccaggttgcctaaggggggtgaaccggcccaggtcggacccgaggagagaatcacttacggatcctcgttaagtggctctcagtcgcccgtaactccgatgccgatgcagcggcgacaactgcacccaaggctcggatcctcgggaacggagcaggtcaaaactcccgtgctg

SEQ ID NO: 35 - RNA, 310 nt, synthetic construct
  source 1..310: mol_type = other RNA
gggagaatcacttacggatcctcgttaagtggctctcagtcgcccgtaactccgatgccgatgcagcggcgacaactgcacccaaggctcggagaaggaaggtttggtatgtggtatattctccgagcccaacacacacacacacacacacacacacacaacacacacacacacacacaagcttctgagctgactgcgatgggagcacgaaacgtcgtggcgcaattgggtggggaaagtccttaaaagagggccaccaagctcagaagctgaaggagaggagaggtagaggagaagcttctgagcttgg

SEQ ID NO: 36 - DNA, 58 bp, synthetic construct
  source 1..58: mol_type = other DNA
ttgtcgccgcagcatcggcaacggagttaccggcgactgacagccacttatcgaggat

SEQ ID NO: 37 - RNA, 26 nt, synthetic construct
  source 1..26: mol_type = other RNA
cggcatcaatatggtgacctcccggg

SEQ ID NO: 38 - RNA, 50 nt, synthetic construct
  source 1..50: mol_type = other RNA
agcgggggaccaccaggttgcctaaggggggtgaaccggcccaggtcgga

SEQ ID NO: 39 - RNA, 28 nt, synthetic construct
  source 1..28: mol_type = other RNA
aacggagcaggtcaaaactcccgtgctg

SEQ ID NO: 40 - RNA, 86 nt, synthetic construct
  source 1..86: mol_type = other RNA
gggcggcgatgagatgactctttgcgataataataacaaaaagggtatttgagccaactcgttcgcctgaccactgcgtgactgcc

SEQ ID NO: 41 - RNA, 85 nt, synthetic construct
  source 1..85: mol_type = other RNA
gggcggcgatgagatgacaaagcgataataagaacaataagggctttctttctgtcgtagctattggtaccactgcgtgactgcc

SEQ ID NO: 42 - RNA, 94 nt, synthetic construct
  source 1..94: mol_type = other RNA
gggagaatcacttacggatcctcccgtctttggactggtccgacaggaaaagcattagaatgtgcgccctggttgatccaaggctcggaagctt

SEQ ID NO: 43 - RNA, 93 nt, synthetic construct
  source 1..93: mol_type = other RNA
gggagaatcacttacggatcccggaaaaataataacgagaagcgaaaagtaagtagattactcctttgcttcggccccaaggctcggaagctt

SEQ ID NO: 44 - RNA, 93 nt, synthetic construct
  source 1..93: mol_type = other RNA
gggagaatcacttacggatccgttttgagacttgctccgattcacaagaaaaataataagaaagttggatcggagtccaaggctcggaagctt

SEQ ID NO: 45 - RNA, 93 nt, synthetic construct
  source 1..93: mol_type = other RNA
gggagaatcacttacggatccttgagaaaaataagaataagtgagtgtcgcgtaagatggtaaatcccttactctcccaaggctcggaagctt

SEQ ID NO: 46 - RNA, 93 nt, synthetic construct
  source 1..93: mol_type = other RNA
gggagaatcacttacggatccttctgccgagttattggtgacgaccttaaaagaataataacaaacgtgtcgttcgccaaggctcggaagctt

SEQ ID NO: 47 - RNA, 93 nt, synthetic construct
  source 1..93: mol_type = other RNA
gggagaatcacttacggatcccggatctttcgatttcagccgaagccttgataataataaagataactggtttgacccaaggctcggaagctt

SEQ ID NO: 48 - RNA, 93 nt, synthetic construct
  source 1..93: mol_type = other RNA
gggagaatcacttacggatcctggagaagacaaattagaatagggcgcccagtgacctgtaagtgaagggcctcctccaaggctcggaagctt

SEQ ID NO: 49 - RNA, 94 nt, synthetic construct
  source 1..94: mol_type = other RNA
gggagaatcacttacggatcccgtttgatctgacggcccttggatagcacgagaattagaacaataagtgtgtttttccaaggctcggaagctt

SEQ ID NO: 50 - RNA, 93 nt, synthetic construct
  source 1..93: mol_type = other RNA
gggagaatcacttacggatccagttacaataataagaacataaacttggattcgtttgtgagcttaccgacttattccaaggctcggaagctt

SEQ ID NO: 51 - RNA, 93 nt, synthetic construct
  source 1..93: mol_type = other RNA
gggagaatcacttacggatccttttatcgagtctaggatgcgacaatcaacaataagaacaataagatctgtcgcgccaaggctcggaagctt

SEQ ID NO: 52 - RNA, 93 nt, synthetic construct
  source 1..93: mol_type = other RNA
gggagaatcacttacggatcctatgacgttcaaaaaataagaataacaaagagcgtcatggttgatctgtcccgggccaaggctcggaagctt

SEQ ID NO: 53 - RNA, 93 nt, synthetic construct
  source 1..93: mol_type = other RNA
gggagaatcacttacggatccttttcttccaagtcttcggacacgactataataacaataagaaaatgttgcccgtccaaggctcggaagctt

SEQ ID NO: 54 - RNA, 93 nt, synthetic construct
  source 1..93: mol_type = other RNA
gggagaatcacttacggatcctttcacgagtcttcgctatgataacaataacaataacgtgtcggttgacacctagccaaggctcggaagctt

SEQ ID NO: 55 - RNA, 93 nt, synthetic construct
  source 1..93: mol_type = other RNA
gggagaatcacttacggatcctctcccttgtccttgggatttcggtaacgataataacaataaaaagggttgtcgaccaaggctcggaagctt

SEQ ID NO: 56 - RNA, 93 nt, synthetic construct
  source 1..93: mol_type = other RNA
gggagaatcacttacggatccgacctctttcctgtcttggcggtatcggtactataataataaaaagcgcatatcgccaaggctcggaagctt

SEQ ID NO: 57 - RNA, 75 nt, synthetic construct
  source 1..75: mol_type = other RNA
gggcgaattcgggttggatagtaggcgcatatggcatcttcgtggttgtgtattgccctttagtgagggttaatt

SEQ ID NO: 58 - RNA, 74 nt, synthetic construct
  source 1..74: mol_type = other RNA
gggccgagaattcaactgccatctaggccttataaacagccggatcccgattgtgctcgatatgtactcggccc

SEQ ID NO: 59 - RNA, 69 nt, synthetic construct
  source 1..69: mol_type = other RNA
gggcattgtgctcgatatgtactccttcgggagaattcaactgccatctaggccttataaacagtgccc

SEQ ID NO: 60 - RNA, 73 nt, synthetic construct
  source 1..73: mol_type = other RNA
gggaggccttataaacagccggatcccgattgtgctcgatatgtactccttcgggagaattcaactgcctccc

SEQ ID NO: 61 - RNA, 100 nt, synthetic construct
  source 1..100: mol_type = other RNA
gggagaattcaactgccatctaggcccacagttcagaggcaccgcgaacaaaacgcaagacagagtgccgacaagagcactacaagcttctggactcggt

SEQ ID NO: 62 - RNA, 65 nt, synthetic construct
  source 1..65: mol_type = other RNA
gggcagaggcaccgcgaacaaaacgcaagacagagtgccgacaagagcactacaagcttctgccc

SEQ ID NO: 63 - RNA, 98 nt, synthetic construct
  source 1..98: mol_type = other RNA
gggagaattcaactgccatctaggcacacgcgagatagagcgaggcctccaaaaatggccacgccaggaagcaagtactacaagcttctggactcggt

SEQ ID NO: 64 - RNA, 101 nt, synthetic construct
  source 1..101: mol_type = other RNA
gggagaattcaactgccatctaggcgacccagggccgggacgcaaagcagccaaaacagacggccccagtcagggagttactacaagcttctggactcggt

SEQ ID NO: 65 - RNA, 83 nt, synthetic construct
  source 1..83: mol_type = other RNA
gaagcttacaagaaggacagcacgaataaaacctgcgtaaatccgccccatttgtgtaagggtagtgggtcgaattccgctca

SEQ ID NO: 66 - RNA, 32 nt, synthetic construct
  source 1..32: mol_type = other RNA
ggatcctgaaactgttttaaggttggccgatc

SEQ ID NO: 67 - RNA, 80 nt, synthetic construct
  source 1..80: mol_type = other RNA
caggctgcatgatggaacagcccataacatacttgaaactgtaaggttggcgtatgcatgaagcgttccatgcatgcctg

SEQ ID NO: 68 - RNA, 55 nt, synthetic construct
  source 1..55: mol_type = other RNA
ggccgcccggcgccatacttgaaactgtaaggttggcgtatggcgccgggcggcc

SEQ ID NO: 69 - RNA, 80 nt, synthetic construct
  source 1..80: mol_type = other RNA
gggacgctcacgtacgctcacgtccgatcgatactggtatattggcgccttcgtggaatgtcagtgcctggacgtgcagt